(19) United States
(12) Patent Application Publication
(10) Pub. No.: US 2024/0189458 A1
(43) Pub. Date: Jun. 13, 2024

(54) METHODS FOR IN VIVO DELIVERY OF MICROBES TO HUMAN MICROENVIRONMENTS

(71) Applicants: William Marsh Rice University, Houston, TX (US); Baylor College of Medicine, Houston, TX (US)

(72) Inventors: Jeffrey TABOR, Houston, TX (US); Omid VEISEH, Houston, TX (US); Robert BRITTON, Houston, TX (US); Moshe BARUCH, Houston, TX (US); Annie GOODWIN, Houston, TX (US); Elena MUSTEATA, Houston, TX (US); Michael DOERFERT, Houston, TX (US); Samira AGHLARA-FOTOVAT, Houston, TX (US); Maxwell HUNT, Houston, TX (US)

(73) Assignees: William Marsh Rice University, Houston, TX (US); Baylor College of Medicine, Houston, TX (US)

(21) Appl. No.: 18/570,949

(22) PCT Filed: Jun. 15, 2022
(86) PCT No.: PCT/US2022/033650

§ 371 Date: Dec. 15, 2023

Related U.S. Application Data
(60) Provisional application No. 63/210,792, filed on Jun. 15, 2021.

(51) Int. Cl. A61K 49/00; A61K 9/48; A61K 9/50; A61K 9/51; A61K 35/74; C12N 11/04; C12N 11/10

(52) U.S. Cl. CPC A61K 49/0008; A61K 9/4808; A61K 9/5015; A61K 9/5036; A61K 9/5094; A61K 9/5115; A61K 35/74; A61K 49/0091; C12N 11/04; C12N 11/10

(57) ABSTRACT

The present disclosure provides compositions comprising encapsulated engineered bacteria. The bacteria may be engineered to act as sensors of biomarkers, such as inflammation, as well as to produce diagnostic or therapeutic agents.

### PRIORITY CLAIM

This application claims benefit of priority to U.S. Provisional Application Ser. No. 63/210,792, filed Jun. 15, 2021, the entire contents of which are hereby incorporated by reference.

### STATEMENT REGARDING FEDERALLY FUNDED RESEARCH

This invention was made with government support under Grant Nos. R01DK120459 and R01AI155586, awarded by the National Institutes of Health, and MCB-1553317, awarded by the National Science Foundation. The government has certain rights in the invention.

### BACKGROUND

### 1. Field

The disclosure relates generally to the fields of biology and medical devices. More particularly, it concerns devices to deliver engineered bacteria to a subject for diagnostic and/or therapeutic purposes.

### 2. Related Art

The mammalian colon (gut) plays important roles in metabolism, and immune and brain function. Gut processes are orchestrated by metabolic and signaling interactions between host cells and the dense and diverse community of resident bacteria (the microbiota). Disruptions in these interactions due to host genetics, environmental agents, or changes to the composition or physiological activity of the microbiota are linked to a spectrum of diseases including obesity, inflammation, cancer, and depression. However, due to the complexity and relative inaccessibility of the gut environment, and the challenges in constructing realistic in vitro gut models, these processes remain poorly understood (Daeffler et al., 2017).

Genetically engineered sensor bacteria have untapped potential as tools for analyzing gut pathways. Bacteria have evolved sensors of a large number of gut-relevant molecules. Such sensors could be repurposed and used to control the expression of reporter genes, enabling minimally invasive measurements of gut metabolites. Thus, there is an unmet need for engineered bacterial sensors that can be delivered in vivo to sense and report inflammation as well as methods of treating diseases associated with the inflammation.

This invention was funded in part by the Robert A. Welch Foundation under Grant No. C-1856.

### SUMMARY

In a first embodiment, the present disclosure provides device comprising an engineered bacteria (e.g., a diagnostic, therapeutic, and/or theranostic bacteria) encapsulated in a hydrogel matrix. In particular aspects, the engineered bacteria is an engineered bacterial biosensor.

In some aspects, the hydrogel matrix comprises alginate. In some aspects, the alginate is present at a concentration of 0.1-10 weight percent, such as 0.1, 0.5, 1, 1.5, 2, 2.5, 3, 3.5, 4, 4.5, 5, 5.5, 6, 6.5, 7, 7.5, 8, 8.5, 9, 9.5, or 10 weight percent, particularly about 1.4 weight percent. In certain aspects, the hydrogel matrix is paramagnetic. For example, the hydrogel matrix may comprise a magnetic small molecule or nanoscale material. In some aspects, the nanoscale material is a metal oxide or metal-based agent. In specific aspects, the metal oxide is titanium oxide, iron oxide, or ferrite. In particular aspects, the metal-based agent is ferric ammonium citrate. In some aspects, the hydrogel matrix further comprises iron oxide. In certain aspects, the hydrogel matrix further comprises an ultraviolet (UV) absorbent small molecule. For example, the UV absorbent small molecule is a dye, photoabsorber, or photoblocker. In particular aspects, the UV absorbent small molecule is tartrazine (Yellow 5). In some aspects, the UV absorbent nanoscale material is a metallic crystal, synthetic polymer, natural polymer, or carbon-based material. In specific aspects, the UV absorbent nanoscale material is titanium oxide. In certain aspects, the UV absorbent molecule is attached to alginate. In certain aspects, the hydrogel matrix is crosslinked with barium cations. In particular aspects, the hydrogel matrix comprises polyacrylamide, PEG, or chitosan. In some aspects, the hydrogel matrix comprises alginate and polyacrylamide. In certain aspects, the polyacrylamide and alginate are present at a ratio of 30:1 to 0.1:1, such as 20:1 to 1:1, particularly about 10:1 to 1:1. In certain aspects, the polyacrylamide and alginate are present at a ratio of 20:1, 10:1, or 5:1. In particular aspects, the hydrogel matrix comprises alginate and PEGDA. In some aspects, the PEGDA and alginate are present at a ratio of 10:1 to 0.5:1, such as 10:1, 9:1, 8:1, 7:1, 6:1, 5:1, 4:1, 3:1, 2:1, 1:1, or 0.5:1, particularly about 1:1. In certain aspects, the hydrogel comprises tetramethylethylenediamine (TEMED). In some aspects, the TEMED is present at a concentration of 0.1% to 30% by volume, such as 0.1%, 0.5%, 1%, 5%, 10%, 15%, 20%, 25%, or 30% by volume. In particular aspects, the TEMED is present at a concentration of 0.1% by volume

In certain aspects, the device comprises an inner core and outer shell. In some aspects, the device is in a sphere confirmation. In specific aspects, the device is in a cylindrical noodle confirmation. In some aspects, the engineered bacteria is in the inner core. In particular aspects, the outer shell is semi-permeable. In some aspects, the outer shell comprises a semi-permeable polymer. In specific aspects, the outer shell is permeable to molecules under 500 kDa.

In some aspects, the hydrogel matrix is chemically modified. In certain aspects, the hydrogel matrix has a specific density of chemical modifications. In specific aspects, the modification is an oligosaccharide, bacteriocidal molecule, bacteriostatic molecule, or a DNA vector that is transmitted into the host cell cytoplasm enabling expression of a gene in the host cell. In some aspects, the chemical modification has a targeted density between 0 and 5 mmol per gram (e.g., 1, 2, 3, or 4 mmol per gram) of alginate in the hydrogel matrix.

In specific aspects, the hydrogel matrix is formulated to promote cell-material interactions between the semi-permeable polymer and the engineered bacteria. In some aspects, the matrix has one or more chemical components to promote cell-material interactions. In certain aspects, the one or more chemical components comprise an adhesive peptide or substrate. For example, the adhesive peptide is fibronectin. In specific aspects, the substrate is xantham gum.

In some aspects, the engineered bacterial biosensor is genetically engineered to sense a physiological chemical or physical signal. In specific aspects, the engineered bacterial biosensor responds to one or more biomarkers of intestinal inflammation. In some aspects, the biomarker of intestinal inflammation is thiosulfate, tetrathionate, nitrate, trimethyl amine N-oxide, calprotectin, kynurenine, reactive oxygen species, reactive nitrogen species, substance P, CCL20, or acidic pH. In particular aspects, the engineered bacterial biosensor comprises a one-component cytoplasmic protein capable of sensing ligands in the cytoplasm. For example, the one-component cytoplasmic protein capable of sensing ligands in the cytoplasm is SoxS or KynR. In certain aspects, the engineered bacterial biosensor comprises a membrane-bound two-component system. In particular aspects, the two-component system comprises ligand-induced signaling and regulation of gene expression by communicating with cytoplasmic proteins. In some aspects, the two-component system detects pH, cadaverine, putrescine, nitrite, sialic acid, cellulose, estradiol, testosterone, androstenedione, or human antimicrobial peptides. In specific aspects, the two-component system is a PhoPQ two-component system. In particular aspects, the PhoPQ two-component system detects human microbial peptides (AMPs), such as RK-31, KS-27, KS-30, TLN-58, LL-29, ALL-38, GNLY, CCL20, TCP, KR-20, CCL-19, hBD3, and/or NPY. In particular aspects, the two-component system is a ThsSR, TtrSR, NarXL, TorTSR, PhoPQ, or SO_4387-SO_4388_{RE}C-PsdR_{DBD} 137 two-component system.

In certain aspects, the engineered bacteria is an Escherichia coli, Lactobacillus reuteri, Lactobacillus crispatus, Lactobacillus rhamnosus, Lactobacillus crispatus, Lactococcus lactis, Bacteroides thetaiotamicron, or Bacillus subtilis bacterium. In some aspects, the engineered bacteria comprises at least two strains of bacteria. In certain aspects, the at least two strains of bacterium can communicate using acyl homoserine lactones, autoinducer peptides, or other interbacterial communication signals. In particular aspects, the Escherichia coli is further defined as Escherichia coli Nissle 1917.

In some aspects, the engineered bacterial biosensor can compute sensory information and respond to the environment outside the construct to activate gene expression directly or activate an engineered genetic circuit that activates gene expression. In some aspects, the engineered bacterial biosensor comprises SO_4387-SO_4388_{REC}-PsdR_{DBD} 137 to detect acidic pH and activate expression of a reporter gene or a T7 RNA polymerase to activates production of the reporter gene. In certain aspects, the engineered bacterial biosensor can respond to a signal by extracellular secretion of a molecule, production of a molecule that diffuses extracellularly, or cell lysis that releases molecules extracellularly. In specific aspects, the engineered bacterial biosensor secretes IL-22 through protein secretion machinery. In particular aspects, the protein secretion machinery comprises the Sec pathway, an acylhomoserine lactone that diffuses across the membrane, or the production of a phage lysin that induces cells lysis to result in spillage of IL-22.

In some aspects, the engineered bacteria expresses a cytokine, anti-NGF, bacteriocin, viral antigen, bacterial antigen, human allergen, monoclonal antibody, nanobody, bacterial quorum sensing molecule, viral vector, DNA, and/or RNA. In certain aspects, the engineered bacteria expresses at least one reporter. In some aspects, the at least one reporter is selected from a chemiluminescent protein, a bioluminescent protein, a fluorescent protein, a colorimetric protein, a pigment-producing enzyme, or a barcoded messenger RNA. In certain aspects, the at least one reporter is sfGFP. In some aspects, the at least one reporter is produced in response to ligand binding. In certain aspects, the engineered bacteria constitutively expresses a second reporter.

In certain aspects, the engineered bacteria expresses a therapeutic agent. In some aspects, the therapeutic agent is a protein. In other aspects, the therapeutic agent is not a protein. In specific aspects, the therapeutic agent is an immune system modulator such as a cytokine, hormone, small molecule, insulin, glucagon, or statin. In specific aspects, the immune system modulator is a cytokine, such as IL-1, IL-1a, IL-1b, IL-2, IL-3, IL-4, IL-5, IL-6, IL-8, IL-9, IL-10, IL-22, IL-28A, IL-19, IL-24, IL-29, IL-20, IL-28β, TGFβ, IL-12, IL-13, IL-16, IL-18, IFNα, IFNβ, IFNγ, TNFα, TNFβ, or IL-35. In some aspects, the engineered bacteria can release the therapeutic agent. In certain aspects, the therapeutic agent has sustained or conditional release. In particular aspects, the therapeutic molecule has substantially non-pulsatile release.

In some aspects, the device comprises 10¹ to 10¹⁰ engineered bacteria. In certain aspects, each engineered bacteria is about 1 microns to about 3 microns in size.

In certain aspects, the device comprises a degradable component. In some aspects, the degradable component is a degradable alginate motif or one or more degradable crosslinkers. In certain aspects, the degradable alginate motif is partially oxidized alginate. In some aspects, the one or more degradable crosslinkers are MMP-sensitive peptide crosslinkers. In some aspects, the degradable component is an innate property of the construct material, a product of the material interacting with the in vivo environment, and/or a product of the construct's cellular components interacting directly or indirectly with the construct material. In some aspects, the degradable component is in the inner zone and/or outer shell. In certain aspects, the degradable component is a degradable chemistry motif integrated into the hydrogel matrix as a part of the polymer and/or as a degradable crosslinker. In specific aspects, the degradable component is degradable is response to pH or an enzyme. In some aspects, the degradable is degradable by the engineered bacteria and/or secreted cellular components. In certain aspects, the device is formulated for oral administration, implantation, inhalation, or injection. In some aspects, the device is formulated for implantation.

A further embodiment provides a method of treating a disease in a subject in need thereof comprising administering an effective amount of the device of the present embodiments (e.g., a device comprising an engineered bacteria (e.g., a diagnostic, therapeutic, and/or theranostic bacterium, such as an engineered bacterial biosensor) encapsulated in a hydrogel matrix to the subject.

In some aspects, the disease is inflammatory bowel disease, Crohn's, ulcerative colitis, vaginal yeast infection, bacterial vaginosis, parasitic infection, appendicitis, lung cancer, acute respiratory distress syndrome, mesothelioma, thrush, or oral cancer. In particular aspects, the parasitic infection trichomoniasis. In some aspects, the disease is Crohn's. In certain aspects, the disease is inflammatory bowel disease. In specific aspects, the disease is ulcerative colitis.

In additional aspects, the method further comprises monitoring inflammation in the subject. In some aspects, monitoring inflammation comprises detecting a reporter produced by the engineered bacteria. In certain aspects, monitoring inflammation does not comprise an invasive procedure, such as a colon biopsy or endoscopy. In some aspects, monitoring inflammation comprises analysis of a fecal sample from said subject and performing flow cytometry to detect said reporter.

Another embodiment provides a method of monitoring inflammation in a subject in need thereof comprising administering an effective amount of the device of the present embodiments (e.g., a device comprising an engineered bacteria (e.g., a diagnostic, therapeutic, and/or theranostic bacterium) encapsulated in a hydrogel matrix) to the subject and detecting a reporter produced by the engineered bacteria. In some aspects, the subject has inflammatory bowel disease, Crohn's, ulcerative colitis, vaginal yeast infection, bacterial vaginosis, parasitic infection, appendicitis, lung cancer, acute respiratory distress syndrome, mesothelioma, thrush, or oral cancer. In particular aspects, the parasitic infection trichomoniasis. monitoring inflammation does not comprise an invasive procedure. In specific aspects, the invasive procedure is a colon biopsy or endoscopy. In some aspects, detecting comprises analysis of a fecal sample from said subject and performing flow cytometry to determine the level of said reporter.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Construct comprising inner zone comprising of 10-10¹⁰ engineered bacteria and an outer zone comprising a shielding that is semi-permeable to molecules within a range of defined molecular weights. Construct can be built into various configurations, such as the cylindrical noodle configuration and the spherical capsule configuration.

FIG. 2: Pore size of the semi-permeable alginate used remains under 500 kDa over the course of 24 hours, assayed by dextran diffusion from inner zone into surrounding media.

FIGS. 3A-3B: Bacteria remain viable when encapsulated within alginate “noodle” formation. (FIG. 3A) Microscopy of noodle shows bacterial fluorescence from constitutive mCherry expression. (FIG. 3B) Equivalent bacterial quantities prior to and following encapsulation exhibit similarly sized populations. CFU/mL of homogenized sections of the alginate noodle showing no variability between each 1-inch section with even distribution of bacteria throughout the noodle. Encapsulation does not affect viability.

FIGS. 4A-4B: Encapsulated bacteria remain viable. (FIG. 4A) Staining with commercially available bacterial viability kit demonstrates bacterial viability post-encapsulation. SYTO9 dye stains all cells, while propidium iodide (PI) stains only non-living cells with damaged cell membranes. (FIG. 4B) Equivalent bacterial quantities plated prior to and following encapsulation exhibit similarly sized populations.

FIG. 5A-5C: Long-term storage at −20° C. does not affect bacterial viability or capsule integrity. (FIG. 5A) Bacterial viability can be preserved using optimized long-term storage procedure. Varying storage procedures and storage buffers were assayed. (FIG. 5B) Bacterial ThsSR sensor response can be preserved using long-term storage procedure. (FIG. 5C) Microscopy analysis suggests that capsules maintain integrity. Images illustrate capsules during the long-term storage procedure that was found to perform best in terms of maintaining integrity. This procedure consists of freezing at −20° C. for 7 days in LB media with 20% glycerol, followed by a 1 hour thaw at 4° C., and 1 hour storage at room temperature. ThsSR sensor bacteria encapsulated within the alginate hydrogels maintain viability (CFU/capsule), and thiosulfate response (MEFL) prior to and after 7-day storage at both 4° C. and −80° C. Bright field microscopy images of thawed hydrogels from various storage conditions.

FIGS. 6A-6E: Sterilization of the capsule surface and storage buffer can be accomplished by 5 min of 365 nm UV exposure at intensity 20 mW/cm², while maintaining viability of encapsulated bacteria. (FIG. 6A) Screen of TiO₂ nanoparticles of varying crystal structure and concentration reveals optimally performing variants for absorbance at 365 nm. The UV source used for the sterilization technique emits at 365 nm. (FIG. 6B) Absorbance spectra of TiO₂ alginate formulations indicate maximum absorbance at the currently utilized wavelength, 365 nm, but also indicate that another wavelength, 260 nm could be used as well. (FIG. 6C) Sterilization technique is capable of killing maximum possible amount of bacteria on capsule surface. Capsule surface was exposed to bacterial load modeling the case where 100% of bacteria to be encapsulated contaminate the surface. (FIG. 6D) TiO₂ nanoparticles of the 30 nm crystal structure (i.e., TO) protect encapsulated bacteria from UV sterilization process. Viability is maintained pre- and post-sterilization. (FIG. 6E) Capsule storage buffer contains minimal bacteria post-encapsulation and can be completely sterilized.

FIGS. 7A-7C: Tartrazine could be used as an additional or alternative UV-protectant material for encapsulation. (FIG. 7A) Screen of tartrazine alginate formulations at varying concentrations reveal optimally performing variants for absorbance at 365 nm. The UV source used for the sterilization technique emits at 365 nm. (FIG. 7B) Absorbance spectra of various tartrazine-alginate solutions, along with two suggested UV wavelengths for sterilization. (FIG. 5C) Microscope image comparison of 0% and 75% tartrazine-alginate capsules

FIG. 8: Schematic of ligand-induced signaling through ThsS/R and plasmid design of the constitutive sensors in the diagnostic strain of Escherichia coli Nissle 1917, referred to hereafter as KD01. The ThsSR two-component system is activated by the small molecule thiosulfate, a proposed biomarker for colonic inflammation. In response, the bacteria produce sfGFP, a green fluorescent protein. This strain also constitutively expresses mcherry, a red fluorescent protein.

FIG. 9: Schematic of genetic constructs expressed in novel engineered bacterial strains that employ different kinds of two-component systems and protein sensors. The bacterial strain containing SO_4387-SO_4388REC-PsdRDBD 137 could function as an in vivo sensor of low pH, the strain containing KynR could function as an in vivo sensor of kynurenine, and the strain containing PhoPQ could function as an in vivo sensor of Mg2⁺, pH, and various peptides (e.g., CCL20, or TCP C-terminal peptide). All engineered constructs may integrate any of the fluorescent protein reporters, constitutive or inducible promoters, terminators, one- and two-component systems, ribosome binding sites, origins, and other genetic parts disclosed herein. All strains were engineered into probiotic E. coli Nissle 1917.

FIGS. 10A-10B: Thiosulfate sensing bacteria function while encapsulated. Thiosulfate response is measured by sfGFP fluorescence, as described in FIG. 8. (FIG. 10A) Thiosulfate dose-response is similar prior to and following encapsulation. (FIG. 10B) Encapsulation maintains fold change in fluorescence associated with thiosulfate response in free cells (i.e., “pre-encapsulation”).

FIG. 11: Encapsulation preserves response of kynurenine sensing bacteria. Kynurenine response is measured by sfGFP fluorescence, as described in FIG. 9. Encapsulation maintains significant fold change in fluorescence associated with kynurenine response in free cells (i.e., “pre-encapsulation”).

FIG. 12: Encapsulation preserves response of TCP C-terminal peptide sensing bacteria. Kynurenine response is measured by sfGFP fluorescence, as described in FIG. 9. Encapsulation maintains significant fold change in fluorescence associated with TCP C-terminal peptide response in free cells (i.e., “pre-encapsulation”).

FIG. 13: Encapsulation preserves response of pH sensing bacteria. pH response is measured by sfGFP fluorescence, as described in FIG. 9. Encapsulation maintains significant fold change in fluorescence associated with response to acidic conditions in free cells (i.e., “pre-encapsulation”).

FIG. 14: Schematic depicting in vivo experimental timeline for demonstrating in vivo efficacy of diagnostic bacterial strain E. coli Nissle 1917 KD01, in the Wistar Rattus norvegicus dextran sulfate sodium (DSS) animal model of colitis.

FIG. 15: Encapsulated diagnostic bacteria can sense colon inflammation in vivo with DSS rat model. The sfGFP fluorescence of diagnostic thiosulfate-sensing bacteria correlates to disease progression in rats, indicated by DAI (Disease Activity Index).

FIGS. 16A-16B: Encapsulation enables survival and growth of engineered bacterial populations in the animal gut. (FIG. 16A) Bacterial populations within capsules increase after passage through animal GI tract. Capsules were administered to animals through oral gavage. (FIG. 16A) Microscopy analysis suggests capsule integrity is maintained after passing through the animal gut.

FIG. 17: Schematic of ligand-induced secretion of the anti-inflammatory cytokine IL-22 in the therapeutic strain of L. reuteri. The sppKR two-component system is activated by the peptide sakacin P inducing peptide, and secretes the anti-inflammatory cytokine IL-22 in response. All constructs engineered for secretion from bacteria may integrate any of the constitutive or inducible promoters, terminators, one- and two-component systems, ribosome binding sites, origins, and other genetic parts disclosed herein or known in the art. For example, the signal peptide (SP) denoted in the schematic shown can be either SP000, SP004, or other peptides that would enable secretion by different cellular export mechanisms. In addition, secretion could be achieved in E. coli as well using either the Flagellar Type III Secretion System, the YebF fusion protein complex, or a Type IV Secretion System for example employing the OmpA signal peptide.

FIG. 18: Encapsulated cells can secrete IL-22. Engineered bacteria contained an inducible construct (pLS103) that regulates secretion of IL-22, as described in FIG. 12. As a negative control, bacteria were also included that contained a construct (pSIP411) lacking the IL-22 gene. Mass of IL-22 secreted was normalized by colony forming unit (CFU) of each bacterial sample. This bacterial strain was tested in the animal model to assess therapeutic effect, as described in FIG. 14. The sensor may be replaced with others enabling conditional activation (e.g., by pH), or the bacteria may be co-cultured with L. sakei to induce expression.

FIG. 19: Schematic depicting in vivo experimental timeline for demonstrating in vivo efficacy of therapeutic, IL-22 secreting L. reuteri strain, in the Rattus norvegicus dextran sulfate sodium (DSS) animal model of colitis.

FIG. 20: Incorporation of a UV-absorbent material into capsules to allow for external UV sterilization while protecting encapsulated cells. Schematic depicts experimental plan from FIG. 6.

FIG. 21: Schematic depiction of the device dimensions in both capsule and noodle form, the device orientation in vivo, and the various attachment/deployment methods.

FIG. 22: Synthesis of tartrazine-alginate.

FIG. 23: Brightfield image of alginate noodle encapsulating mammalian cells and a suture. Image taken at 4× magnification; inlay taken at 10× magnification.

FIGS. 24A-24D: Porting S. Typhimurium PhoPQ into E. coli. (FIG. 24A) S. Typhimurium PhoPQ natively controls many physiological functions. (FIG. 24B) Genetic diagram of KB1, in which S. Typhimurium PhoPQ has been ported to laboratory E. coli, and of pKB233, the P_{virK} fluorescent reporter plasmid. (FIG. 24C, FIG. 24D) PhoPQ induction with LL-37 in S. Typhimurium (FIG. 24C) and engineered E. coli KB1 (FIG. 24D) strains containing pKB233, as measured by flow cytometry. Data was collected over n=3 separate days, with results from each day shown as a separate marker.

FIGS. 25A-25D: Surface-displayed AMPs activate PhoPQ in E. coli. (FIG. 25A) Schematic of peptide surface display and genetic diagram of peptide surface display and mNeonGreen fluorescent reporter plasmids. (FIG. 25B) Structure of the only known non-activating human AMP (PDB ID-HNP-1: 3GNY). (FIG. 25C) All available structures of known PhoQ-activating AMPs (PDB ID-cecropin P1: 2N92, protegrin-1: 1PG1, melittin: 6DST, LL-37: 2K6O). (FIG. 25D) PhoPQ activation in KB1 in response to IPTG induction of surface-displayed AMPs, as measured by flow cytometry. Data was collected over n=3 separate days, with results from each day shown as a separate marker.

FIGS. 26A-26F: Discovery and characterization of PhoPQ-activating human AMPs. (FIG. 26A) SLAY-TCS workflow. (FIG. 26B) Sort gate strategy for sort-seq, with sorting bins represented by alternating light and dark grey bars. Histogram represents the results from a single biological replicate (n=1). (FIG. 26C) Calculated fluorescence distributions of no peptide, negative (defensin HNP-1), and positive (LL-37) control strains included in the library. Vertical lines indicate estimated mean fluorescence values for each control. (FIG. 26D) Sort-seq fold activation relative to no peptide control strains for all human AMP activators with >2-fold activation. (FIG. 26E) Net charge and hydrophobicity, as measured by grand average of hydropathicity (GRAVY), for all human AMPs included in the screen. Human AMPs belonging to the same cluster are represented by a single data point with error bars indicating the median and 25^{th} to 75^{th} percentile ranges for that cluster. (FIG. 26F) Performance of the cathelicidin sparse robust linear model on peptide variants of human AMP activators of PhoPQ (cathelicidin, CCL20, hBD3, CCL19, NPY, GNLY, TCP) and on peptides variants of non-activating human AMPs (Other). Coefficient of determination (r²) and Pearson correlation coefficient (r) values were calculated for log₁₀-transformed fold change and predicted fold change values.

FIGS. 27A-27C: (FIG. 27A) Conventional hydrogel encapsulation of bacteria leaves some free external bacteria and also allows for gradual bacteria escape. (FIG. 27B) Schematic for method of using UV sterilization and chemical reinforcement to increase biocontainment of the bacteria while also maintaining platform factors for GI tract delivery (i.e., small capsule size, scalability). (FIG. 27C) Confocal images of capsules made with and without bacteria to show residual bacteria that are on the capsule surface. Images were manually zoomed in to show bacteria that are present on the external capsule surface after capsule formation.

FIGS. 28A-28B: UV absorbance screening of TiNP-alginate formulations. (FIG. 28A) Formulations were first screened across the entire UV light spectrum to find key wavelengths of max absorbance. (FIG. 28B) UV absorbance was tested at 365 nm specifically while varying the TiNP concentration that was suspended in the alginate.

FIGS. 29A-29B: Hydrogel capsules and strings formed with TiNP-Alg. (FIG. 29A) Two sizes of capsules and hydrogel strings were formed by electrospraying the optimal TiNP-Alg formulation (30 nm rutile) into barium crosslinking bath. (FIG. 29B) Capsules are designed with a core-shell structure that can contain engineered bacteria within the core. Images represent an overlay of brightfield and fluorescence microscopy images of capsules. Bacteria are engineered to produce mCherry, a fluorescent red protein, for ease of visualization.

FIG. 30: Diffusion of various size FITC-dextrans from alginate and TiNP-Alg capsules. n=5 per group, data shown as mean+/−s.e.m. Different size FITC-dextran molecules were encapsulated in alginate and TiNP-alginate. The incubation buffer was sampled at various time points to assess the escape rate of the various size fluorescent molecules from the two capsule formulations. Overall, TiNP addition had minimal effect on the diffusion properties of the overall alginate hydrogels.

FIGS. 31A-31D: (FIG. 31A) Kill curve data for free bacteria sterilization with 365 nm UV light. Various known concentrations of bacteria were exposed to one of four UV light doses (5-minute exposure at different intensities). The bacteria suspensions were then plated to assess post-UV viability via CFU. This allowed for the identification of a suitable sterilizing dose of UV for the bacteria and light setup. (FIGS. 31B-31C) UV sterilization of TiNP capsules mixed with known bacteria concentrations. Empty TiNP-alginate capsules were added to known concentrations of bacteria suspension in media before exposing all groups to a sterilizing dose of UV light (6 J/cm² at 365 nm). Immediately after UV exposure, all groups were placed in an incubator for a 24-hour growth period. This was to allow for potential recovery of bacteria after UV as well as to create a higher bar for maintaining sterility. Media (FIG. 31B) and homogenized capsules (FIG. 31C) were plated at 24 hours to assess bacteria presence and viability, which was zero across the board for UV-exposed groups. This showed the sterilization procedure allows for total external sterilization of capsules and their incubation media. (FIG. 31D) Empty capsules were exposed to high density bacterial cultures, sterilized with the UV treatment, and incubated in media for 24 hours. Media was plated for CFU to assess efficacy of sterilization. Capsules were homogenized and plated for CFU to assess efficacy of capsule surface sterilization.

FIG. 32: Impact of UV sterilization on encapsulated cell viability. n=5 per group, data shown as the mean. Engineered bacteria (mCherry-expressing E. Coli NIssle 1917) were encapsulated in alginate and TiNP-alginate. Capsules from both groups that received no UV exposure had high viability of encapsulated bacteria (capsules were homogenized in order to plate their contents for CFU), suggesting that the TiNP has minimal short-term toxicity for the encapsulated bacteria. UV-exposed alginate capsules were completely sterilized and continued to have zero cell viability after 24 hours of growth, showing complete sterilization and no recovery. In contrast, cells encapsulated in TiNP-alginate capsules had good viability immediately after UV exposure and showed further growth over the 24-hour period, suggesting they were successfully shielded from UV by the TiNP inclusion.

FIG. 33: Confocal imaging of capsules with and without UV sterilization. The encapsulated bacteria are mCherry-expressing E. coli Nissle 1917, which will fluoresce red while alive and gradually cease to fluoresce red when dead (UV exposure disrupts DNA structure but does not lyse the cells, meaning the red color will not immediately disappear). Minimal differences can be seen in the alginate or TiNP-alginate capsule groups at zero hours post-UV. The alginate group shows significant bacteria growth by 24 hours (as shown by the formation of colonies in the hydrogel matrix) for the unexposed group and complete cell death for the exposed group. The TiNP group showed minimal differences at 24 hours, with both the unexposed and exposed capsules showing bacteria growth on the surface

FIG. 34: Sterility of capsule incubation media at 24 hours. n=5 per group, data shown as the mean. This is the quantification of bacteria growth in the media at 24 hours. This data verifies that there was total sterilization of the UV-exposed alginate group, as well as viable bacteria growth in both TiNP groups. The growth in media seen for the UV-exposed TiNP group indicates the escape of bacteria from the inside of the capsules since the UV sterilization procedure was designed and verified to completely sterilize the outside of capsules and media. Despite post-UV escape of the encapsulated bacteria over time, the UV sterilization procedure led to significantly lower bacterial growth in the media for both groups, suggesting the sterilization process could help play a role in the biocontainment of the bacteria.

FIGS. 35A-35C: Reinforced alginate hydrogel formulations used to make capsules via electrospray and assessed for biocontainment potential. Several alginate-based alternative hydrogel formulations were made to enable increased post-UV biocontainment of the bacteria. (FIG. 35A) Several variations on PAAm-alginate were tried due to the reagents' known cytotoxicity. (FIG. 35B) Scheme for the full library of 156 hydrogel formulations. A library of alginate-based hydrogel formulations was designed to improve biocontainment of encapsulated bacteria compared to the previous formulation (here designated “A1”) used for this application. All formulations included UV-absorbent TiO₂ NP to protect encapsulated cells from death during sterilization protocol. (FIG. 35C) Details of screened hydrogel formulations.

FIG. 36: Two-step process for screening hydrogel formulations for biocontainment. All capsule formulations were made via electrospraying, were UV-exposed with the previously established dose, and were incubated in media for 72 hours. The first screening step involved checking the optical density of the incubation media to assess growth. Any groups that had an OD at or below the background of media were then plated for viability to ensure their media sterility wasn't due to total cell death from the encapsulation chemistries.

FIG. 37: Bacterial growth in media for all screened hydrogel formulations at 72 hours. n=3 per group, data shown as mean+/−st.dev. The optical density of the incubation media was checked as a quick method of assessing bacterial growth, where a low OD was desirable since it indicated biocontainment of the encapsulated bacteria. Eight groups, all of which were PAAm-alginate formulations, passed this bar for low bacteria growth.

FIG. 38: 72-hour viability of encapsulated cells for low-OD₆₀₀ hydrogel formulations. n=5 per group, data shown as mean+/−st.dev. The eight capsule groups that had low bacteria growth in the media were homogenized to assess the viability of the encapsulated bacteria. Most groups showed zero cell viability, owing to the cytotoxic reagents and chemistry of PAAm, but one group had high cell viability that was comparable to the alginate only group.

FIGS. 39A-39D: Summary of exemplary hydrogel formulation's composition and chemistry (APa13). The formulation is a 5:1 mixture of PAAm: alginate where the alginate is ionically crosslinked with barium chloride, and the PAAm was formed at a relatively low concentration and with minimal crosslinker (TEMED) and no accelerant (APS). (FIG. 39A) The formulation, Ape13, is based on a mixture of barium-crosslinked alginate and covalently crosslinked polyacrylamide, with TiO₂ NP suspension. Simultaneous ionic alginate crosslinking and polyacrylamide formation enables the formation of an interpenetrating polymer network. This formulation was used to generate multiple form factors to improve biocontainment capabilities, specifically generating capsules containing either one or two shells with variable size cores. Bacteria are loaded only into capsule cores, shown in dark blue. The cores and first shell contain TiO₂ NP. The second shell contains only alginate. (FIG. 39B) Bright-field microscopy of single and multi-shell capsules. Bacteria were encapsulated within four hydrogel formulations (A1, A1-MS, APa13, APa13-MS). (FIG. 39C) Hydrogel formulation APa13-MS performs best to prevent bacterial escape from the capsules into the media, with 7 out of 10 replicates remaining completely sterile. Each replicate contained 50 capsules incubated in 1 mL of media. Capsules were incubated for 72 hours and all surrounding media was plated for CFU to quantify bacterial population. (FIG. 39D) Viability of encapsulated bacteria was similar among formulations prior to UV treatment and also following 72 hours of growth post-UV treatment. Encapsulated bacteria are able to grow in population for all hydrogel formulations shown.

FIGS. 40A-40C: (FIG. 40A) Post-UV bacteria growth for core-shell and multi-shell alginate capsules. n=5 per group, data shown as mean+/−s.e.m. **p<0.01. ND=not detected. The PAAm-alginate formulation (APa13) was used to encapsulate bacteria along with TiNPs and was tested for post-UV biocontainment potential against TiNP-alginate. After a 72-hour post-UV growth period, the PAAm-alginate formulation showed significantly lower bacteria growth in the media, suggesting significantly better biocontainment. This further improved the increased biocontainment caused by the UV sterilization process and the comparable viability of the two groups above indicates that the lower bacteria growth is due to better containment rather than the chemistry involved simply killing the cells and allowing for less escape. (FIG. 40B) Spectral screen of TiO₂ nanoparticles of varying crystal structures suspended in alginate. TiO2 NPs absorb UV light at 365 nm. Degree of absorbance and absorbance spectra varies with crystal structure. (FIG. 40C) Absorbance of TiO₂ nanoparticles at 365 nm is greatest for rutile 30 nm crystal structure at high weight percent concentration (0.05 wt. %) in alginate.

FIG. 41: Diffusion of various size FITC-dextrans from alginate and PAAm-Alg capsules. n=5 per group, data shown as mean+/−s.e.m. The diffusion assay was used to assess the impact of the PAAm incorporation on the capsule diffusion properties. The PAAm-alginate formulation showed much lower diffusion of all size molecules at most time points, with the exception being the gradual escape of the 150 kDa FITC dextran at later time points. This suggests the overall porosity of the PAAm-alginate is lower, which indicates a reason for the better containment of the encapsulated cells.

FIGS. 42A-42F: Bacteria can be encapsulated within alginate hydrogels supplemented with magnetic iron oxide nanoparticles to improve ease of retrievability. (FIG. 42A) Brightfield image of encapsulated bacteria. (FIG. 42B) Schematic depicting magnetic-aided capsule retrieval process. (FIG. 42C) Bacterial viability (CFU) within capsules prior to oral administration and following retrieval from stool samples. (FIG. 42D) Activation of ThsSR in sensor bacteria following DSS-induced gut inflammation for n=4 rats. (FIG. 42E) Fluorescence values from retrieved ThsSR sensor bacteria correlate with animal disease state as measured by disease activity index (DAI) at the time of capsule administration. (FIG. 42F) Representative histological sections from rat 5, which was at DAI 6 at the study endpoint.

FIGS. 43A-43B: (FIG. 43A) L. reuteri engineered to secrete human interleukin-10 (hIL-10) via a sakacin-inducible two-component system. (FIG. 43B) hIL-10 secretion from free bacteria under uninduced and induced conditions.

FIGS. 44A-44B: (FIG. 44A) L. reuteri engineered to secrete hIL-10 can be encapsulated within a semipermeable hydrogel wherein the hIL-10 can freely diffuse into the surrounding environment. (FIG. 44B) hIL-10 secretion from encapsulated bacteria under uninduced and induced conditions.

FIG. 45: E. coli Nissle 1917 were engineered to constitutively secrete either mIL-2, mIL-7, mIL-12, or mIL-15 via fusion to the natively exported protein YebF.

FIG. 46: Constitutive mIL-2 secretion from free E. coli Nissle 1917 engineered bacteria. mIL-2 concentration in the bacterial media increases as the bacteria grow.

FIG. 47: Constitutively expressed KynR one-component system engineered in E. coli Nissle 1917 to respond to kynurenine levels via CyPet production. mCherry is produced constitutively to facilitate identification of engineered bacteria among native microbiota via flow cytometry. This sensor exhibits a 5.89-fold change in vitro response to 0.1 mM kynurenine.

FIG. 48: Constitutively expressed KynR sensor was administered to rats prior to and following chemically induced inflammation via DSS treatment. Sensor activation as measured by CyPet fluorescence was significantly higher in DSS-treated rats. CyPet fluorescence correlated with rat disease severity as measured by Disease Activity Index (DAI). Constitutive mCherry fluorescence did not significantly vary with DSS treatment. Constitutive mCherry fluorescence did not significantly correlate with DAI.

FIG. 49: Schematic illustration of hydrogel encapsulated biosensor delivery to the GI. Encapsulated biosensor bacteria are delivered to the digestive tract, where they sense and respond to a biomarker of inflammation. Bacterial viability and hydrogel integrity are maintained during passage through the gastrointestinal tract, such that hydrogels may be retrieved non-invasively and processed to obtain fluorescent readout for diagnosis.

FIGS. 50A-50D: Fabrication and characterization of tunable hydrogel spheres. (FIG. 50A) Schematic of capsule synthesis. (FIG. 50B) Chemical structure of alginic acid depicting associated pKa of each subgroup. (FIG. 50C) Capsules can be reproducibly synthesized with varying diameters by adjusting electrospray voltage. (FIG. 50D) Capsule permeability over 24 hours remains restricted to molecules under 500 kDa in size.

FIGS. 51A-51E: Encapsulation preserves bacterial viability. (FIG. 51A) Representative dark-field image of hydrogel capsules with and without encapsulated E. coli at varying cell densities. (FIG. 51B) Bacterial viability is preserved within capsules. Living bacteria accept only SYTO-9 stain while non-living bacteria accept both SYTO-9 and propidium iodide. Capsule microscopy images are representative of n=3 biological replicates containing 5 capsules each. (FIG. 51C) Bacterial viability (CFU) prior to and following encapsulation per volume encapsulated was not significantly different (p=0.715***). Average bacterial load per capsule is 7.47×10⁴±3.74×10⁴ CFU. (FIG. 51D) Bacteria encapsulated at OD 0.1 can grow inside capsules and reach an average carrying capacity of 2.38×10⁷±1.19×10⁷ CFU/capsule. Encapsulated bacteria were grown for 48 hours, and 5 capsules were homogenized at each timepoint. Experiment was repeated for n=3 biological replicates. (FIG. 51E) Encapsulated bacteria from FIG. 51D were retrieved at 3 timepoints and stained with DAPi to visualize degree of growth within the capsules.

FIGS. 52A-52B: Encapsulation preserves performance of thiosulfate biosensing E. coli Nissle strain. (FIG. 51A) The bacterial two-component system ThsSR is engineered as an in vivo diagnostic tool in probiotic E. coli (Daeffler et al. 2017). Schematic depicts signaling through ThsSR, by which the cell detects thiosulfate and responds by producing a green fluorescent protein (sfGFP). (FIG. 51B) Thiosulfate dose-response is preserved following encapsulation of ThsSR sensor bacteria. Dynamic range, K_{1/2}, and hill coefficient are similar prior to and following encapsulation.

FIGS. 53A-53D: Capsules maintained integrity and bacterial viability during passage through GI tract. (FIG. 53A) Capsules are administered to rats by oral gavage and retrieved after 12 hours from stool samples. Bacteria are retrieved by homogenizing the capsules and contents are plated to assess internal bacterial viability. (FIG. 53B) Encapsulated bacteria survive in acidic conditions. (FIG. 53C) Bacterial viability (CFU) within capsules prior to oral administration and following retrieval from stool samples. (FIG. 53D) Qualitative assessment of capsule integrity via dark-field microscopy reveals consistent integrity prior to and following passage through rat GI tract. Change in color represents a higher density of bacteria present in the sample recovered from stool.

FIGS. 54A-54D: Encapsulated bacteria can diagnose colon inflammation in rat DSS model of inflammation. (FIG. 54A) Timeline of disease development and capsule dosing in rats. Rats are dosed with encapsulated sensor bacteria prior to disease onset by DSS administration. At day 0 they are administered water with 3-5% DSS for 12 days. Sensor bacteria are given by oral gavage following DSS administration. After 12 hours, samples are collected from the rats, processed, and analyzed by flow cytometry to measure sfGFP production. (FIG. 54B) ThsSR in sensor bacteria is activated by gut inflammation for n=10 rats. (FIG. 54C) Fluorescence values from retrieved ThsSR sensor bacteria correlate with animal disease state as measured by disease activity index (DAI) at the time of capsule administration. (FIG. 54D) Representative histological sections from rats with varying degrees of inflammation and different DAI. Rats were sacrificed at day 11 and colon sections were processed. At this point, rats had reached different final DAI states.

FIG. 55: Transfer function parameters for sensor bacteria prior to and following encapsulation. Here, F is the fluorescence at a given ligand concentration L, k₁₂ is the concentration of ligand that elicits a half-maximal response, n is the Hill coefficient, A is the fit of the minimum response with no ligand, and B is the fit of the maximum response at saturating ligand concentration. D is the dynamic range measured by the ratio between the maximal and minimal response.

FIG. 56: Capsules with different shell thicknesses can be reproducibly synthesized by varying the core:shell flow rate ratios of the alginate.

FIGS. 57A-57C: Optimization of bacterial retrieval protocol. (FIG. 57A) Bacteria can survive in EDTA at 4 C for a limited time, so each homogenization step must be performed within 5 minutes. (FIG. 57B) The chelating agent EDTA is necessary for complete release of bacteria from the capsules. (FIG. 57C) Use of the homogenizer is needed to break up the hydrogel capsules and does not affect cell viability of free cells.

FIGS. 58A-58B: Validation of live-dead stain with free and encapsulated cells. (FIG. 58A) Free (i.e., unencapsulated) cells are stained as expected based on viability. SYTO-9 stains only living cells when used independently. Propidium iodide (PI) stains only nonliving cells when used independently. A mixture of both stains can be used to differentiate living from non-living cells; all cells will fluoresce green (SYTO-9), but only non-living cells will fluoresce red (PI). Microscopy images are representative of n=3 biological replicates that were independently stained. (FIG. 58B) Capsules from the samples imaged in FIG. 50B were homogenized and plated to determine viability at time of staining. Living E. coli capsules contained an average population of 5.55×10⁴ CFU, while non-living, heat-killed E. coli capsules contained an average population of 1.29×10³ CFU.

FIG. 59: Expression of constitutive mCherry is not affected by thiosulfate concentration. The mCherry fluorescence of all samples from FIG. 52B was measured and found to remain constant for both free and encapsulated cells.

FIGS. 60A-60D: Effect of DSS on thsSR sensor performance. Sensor function was assayed with no ligand and saturating ligand (5 mM). (FIG. 60A) ThsSR sensor performance in free cells (pre-encapsulation) with and without 5% DSS. (FIG. 60B) mCherry fluorescence of ThsSR sensor free cells (pre-encapsulation) with and without 5% DSS. (FIG. 60C)) ThsSR sensor performance in encapsulated cells (post-encapsulation) with and without 5% DSS. (FIG. 60D) mCherry fluorescence of ThsSR sensor encapsulated cells (post-encapsulation) with and without 5% DSS.

FIGS. 61A-61C: Optimizing bacterial viability within capsules prior to gavage and upon retrieval from stool. (FIG. 61A) Microscopy depicts capsule morphology is not affected significantly by a 2 hour incubation in media at pH 3.2. (FIG. 61B) Capsule integrity was maintained after passage through the GI tract for bacteria administered with sodium bicarbonate or only with PBS or saline. (FIG. 61C) Bacterial viability within capsules after passage through the GI tract is maintained only for samples administered with sodium bicarbonate. Data points represent the net change in capsule population compared to the average population prior to gavage.

FIGS. 62A-62C: Disease progression during DSS administration. (FIG. 62A) Disease progression per rat measured by Disease Activity Index (DAI) over the course of a 12-day DSS dosing regimen. (FIG. 62B) Body weight of individual rats plotted over the course of DSS treatment. (FIG. 62C) spleen weights of individual.

FIGS. 63A-63B: The effect of DSS administration on sfGFP fluorescence and gated cell count of ThsSR sensor bacteria. (FIG. 63A) sfGFP fluorescence from bacteria retrieved from rats prior to DSS is lower than that from bacteria retrieved from rats following DSS. Three capsule samples were collected per animal for n=10 animals. (FIG. 63B) Total gated cell count obtained from each of 3 samples from n=10 rats. Horizontal bar represents 250 count cutoff, datapoints below this are not represented by the means reported per animal in the reported fluorescence data. Following flow cytometry data acquisition, raw data were processed using FlowCal. A standard curve was generated from calibration beads to transform arbitrary units into absolute fluorescence units (MEFL or MECY). Data was then gated by an FSC/SSC scatter profile characteristic of E. coli Nissle 1917. Remaining cell counts were further gated to retain only counts with an FL3 value greater than 5000 MECY. Samples giving fewer than 250 counts by these gating procedures were discarded. Capsules retrieved from DSS-treated rats gave more counts/sample and usable samples than those retrieved from rats prior to DSS treatment. Overall, from 60 samples collected, 49 samples were retained by these criteria (81.26%).

FIG. 64: Complete ThsSR sensor bacteria 2D flow cytometry data per stool sample collected prior to DSS. mCherry vs. sfGFP scatter plots for rats prior to DSS treatment. Data has been gated by an FSC/SSC scatter profile characteristic of E. coli Nissle 1917. Data has been gated to select only cells with mCherry fluorescence higher than 5000 MECY. All capsule samples are shown, including those that were discarded due to low gated cell count. R1-3 represent the 3 replicates collected per rat. Each replicate contains the bacterial contents of 5 capsules.

FIG. 65: Complete ThsSR sensor bacteria 2D flow cytometry data per stool sample collected post-DSS. mCherry vs. sfGFP scatter plots for DSS-treated rats. Data has been gated by an FSC/SSC scatter profile characteristic of E. coli Nissle 1917. Data has been gated to select only cells with mCherry fluorescence higher than 5000 MECY. All capsule samples are shown, including those that were discarded due to low gated cell count. R1-3 represent the 3 replicates collected per rat. Each replicate contains the bacterial contents of 5 capsules.

FIG. 66: Encapsulated red fluorescent bacteria were incubated in a culture of green fluorescent bacteria to qualitatively assess sterilization capabilities. Buffer exchange washes remove many of the external bacteria, and coupled with the UV treatment are able to completely remove bacteria from the surrounding capsule media and capsule surface. The two separate colors allow for visual identification of the introduced external bacteria (green) and encapsulated bacteria (red) before and after various sterilization procedures. Specifically, a procedure with six post-encapsulation buffer washes was compared with the optimized UV sterilization procedure, which includes two washes before UV exposure and one wash after. This model supports the importance of UV sterilization to remove all external bacteria, as the six buffer changes conventionally used in this protocol are not sufficient to fully remove the external bacteria.

FIG. 67: Assay to investigate whether TiO2 alginate formulation protects encapsulated cells from cell death during UV-based sterilization. Schematic of experimental protocol to evaluate sterility of surrounding capsule media and viability of encapsulated bacteria. Engineered red fluorescent bacteria were encapsulated, sterilized with the UV treatment, and incubated in media for 24 hours. Capsule buffer was plated for CFU. Capsules were homogenized and plated to assess viability of internal bacteria.

FIGS. 68A-68B: A secondary encapsulation step is used to cover the capsule formulation in a second shell. (FIG. 68A) Two separate encapsulation procedures are used to generate multishell capsules, with the first creating small core-shell capsules with reinforced PAAm-Alg and TiNPs and the second coating multiple inner capsules in a larger alginate shell. (FIG. 68B) Several ratios of alginate to inner capsules were made to optimize the multishell capsule formation. A ratio of 7:1 alginate to inner capsules was used for the main text figures and work.

FIG. 69: Biocontainment capabilities of exemplary formulations. Each replicate represents 50 capsules incubated in 1 mL of media for 72 hours. All media was plated for CFU to quantify any bacterial escape into the media. All 3 replicates of multi-shell formulation CC were completely sterile and remained bio-contained, while 2 out of 3 replicates of single-shell formulation C remained bio-contained. Group Z represents a sterile media control containing no capsules but processed and incubated similarly to all other groups.

FIG. 70: Variability during electrospray can enable post-sterilization escape of encapsulated bacteria and justifies a secondary shell layer. Variability during the electrospray encapsulation process can cause some of the capsule cores to be abnormally close to the capsule exterior despite designing for a thick capsule shell. Bacteria can gradually escape through this narrow hydrogel barrier and grow externally, regardless of external sterilization procedures.

FIG. 71: FITC-dextran diffusion from control and exemplary capsule formulations. Fluorescent FITC-dextran of 3 molecular weights, 2000, 500, and 150 kDa, were encapsulated group A control capsules (labelled Alg) and exemplary group CC formulation capsules (labelled PAAmAlg). PAAmAlg capsules exhibited slower diffusion of all MW FITC-dextran, suggesting lower porosity compared to Alg capsules. PAAmAlg capsules permitted diffusion of all MW FITC-dextran at or below 500 kDa, supporting its use for the desired in vivo applications where biomarkers and bacterial products will need to cross the hydrogel membrane. Each value represents the average of 5 replicates. Each replicate represents 5 capsules in 100 μL of buffer. Fluorescence values were background subtracted and normalized to the 0 hr timepoint measurements for each formulation.

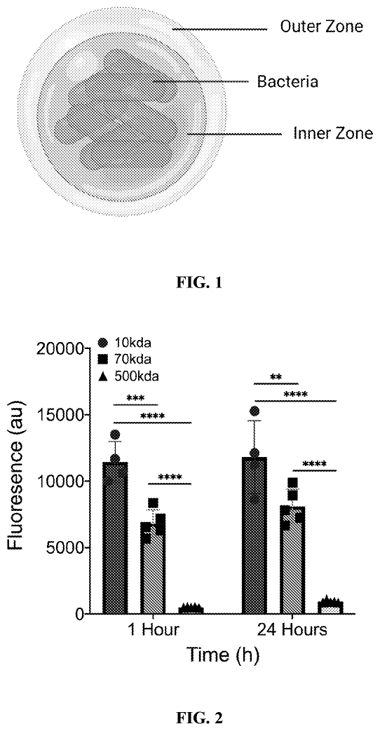

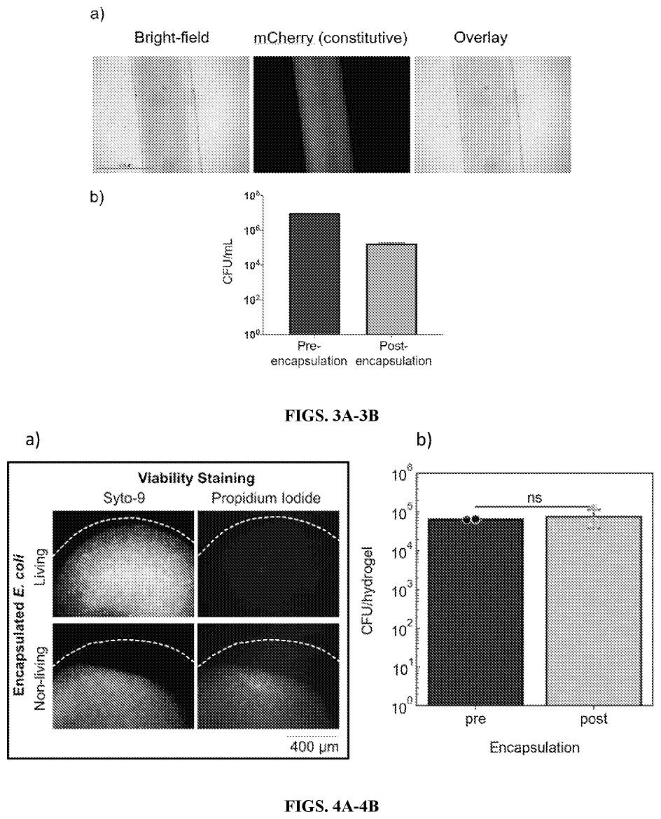

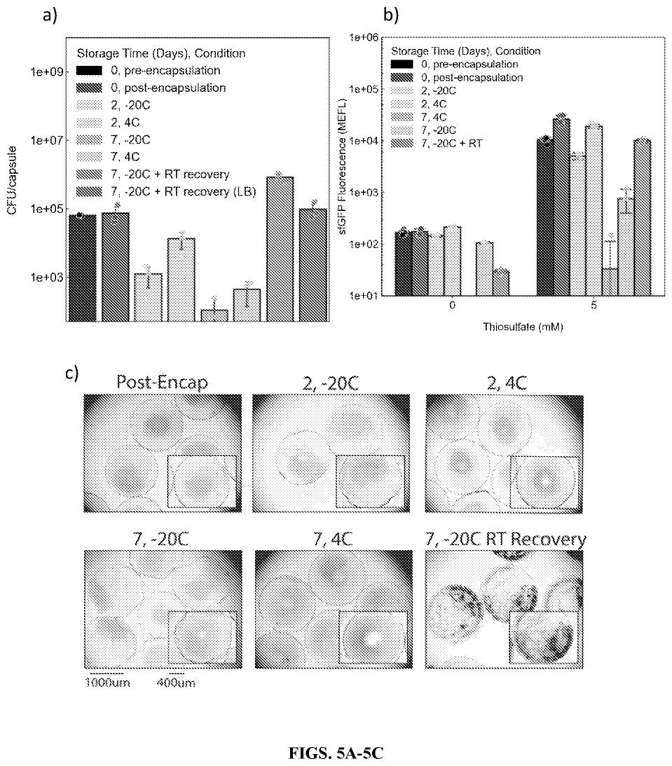

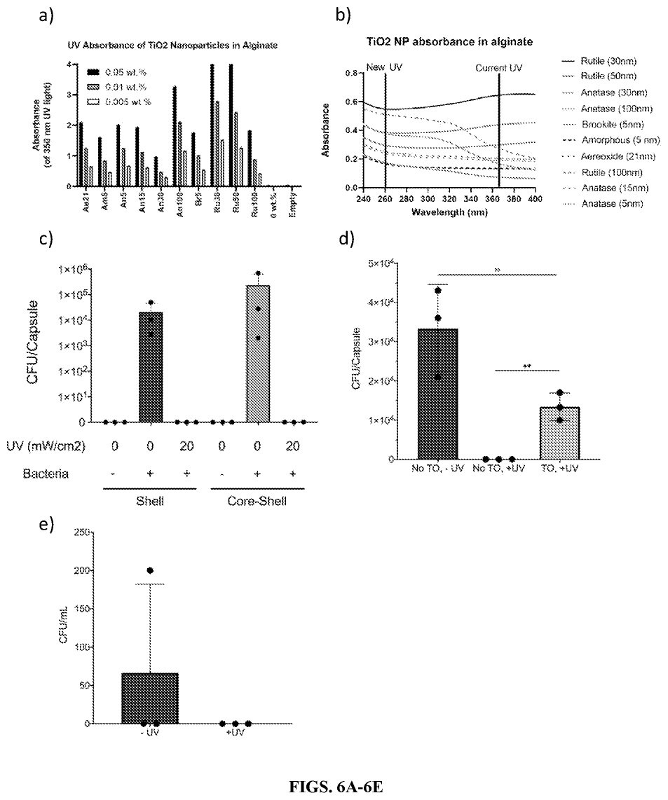

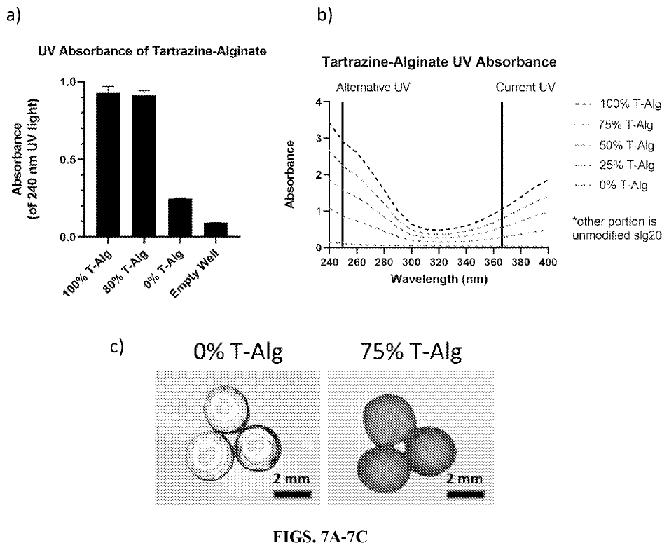

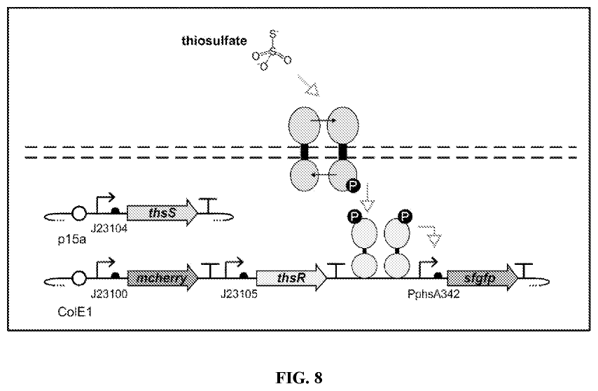

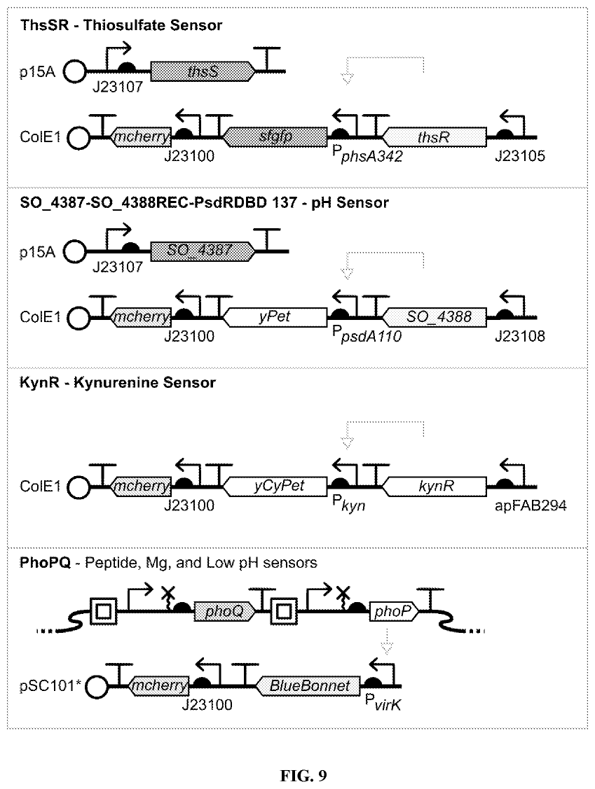

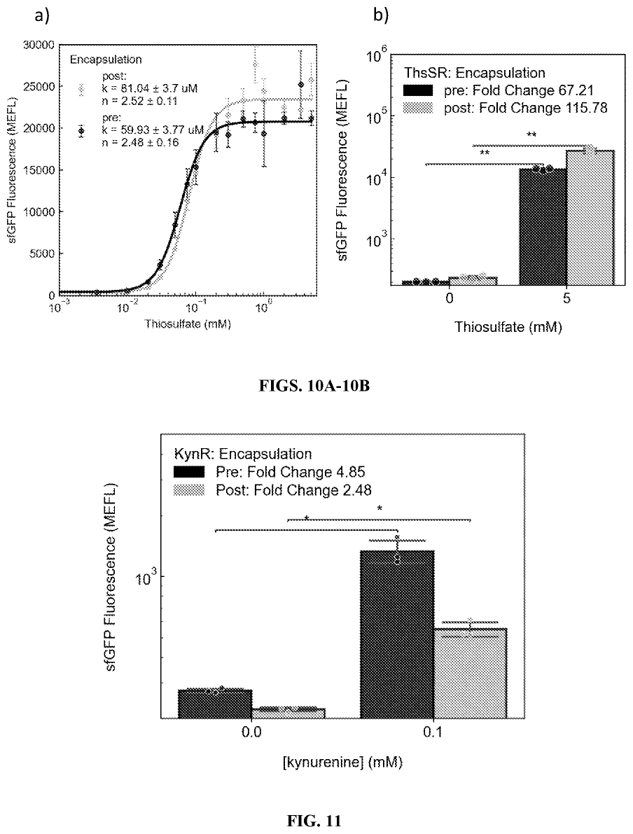

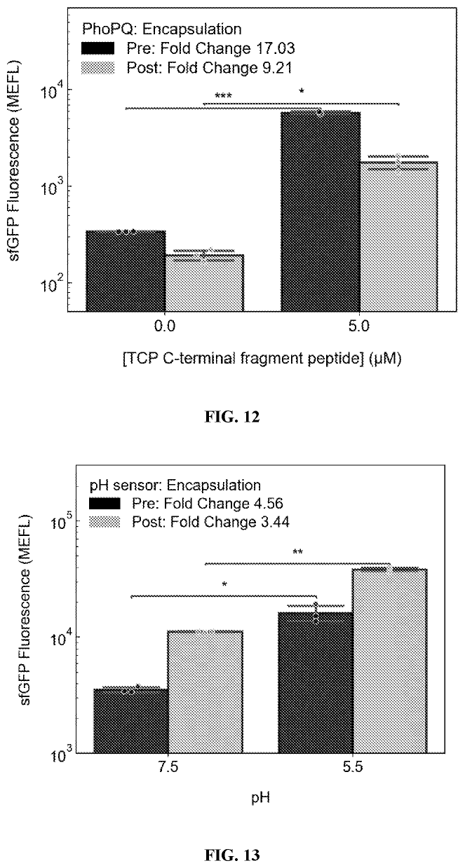

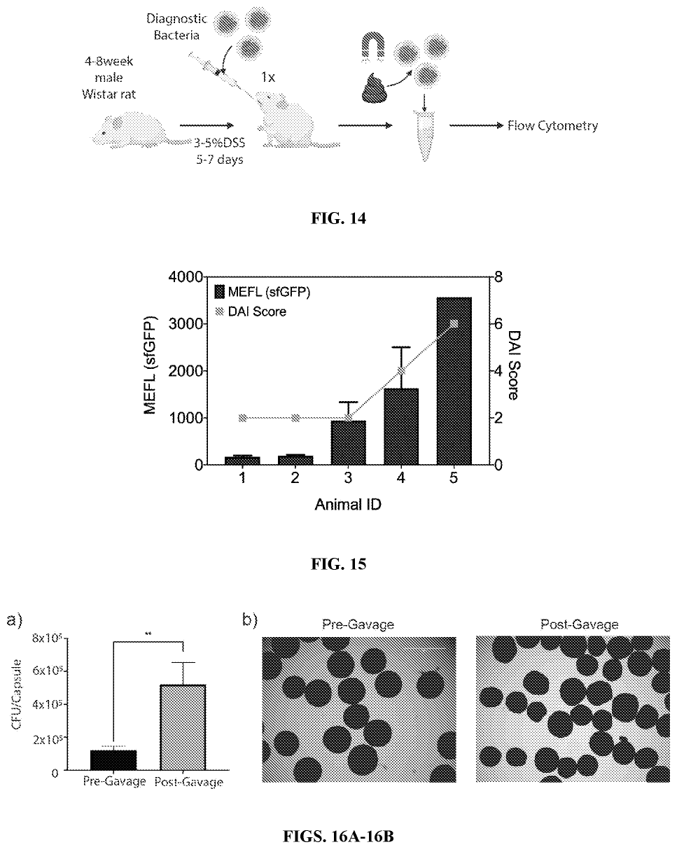

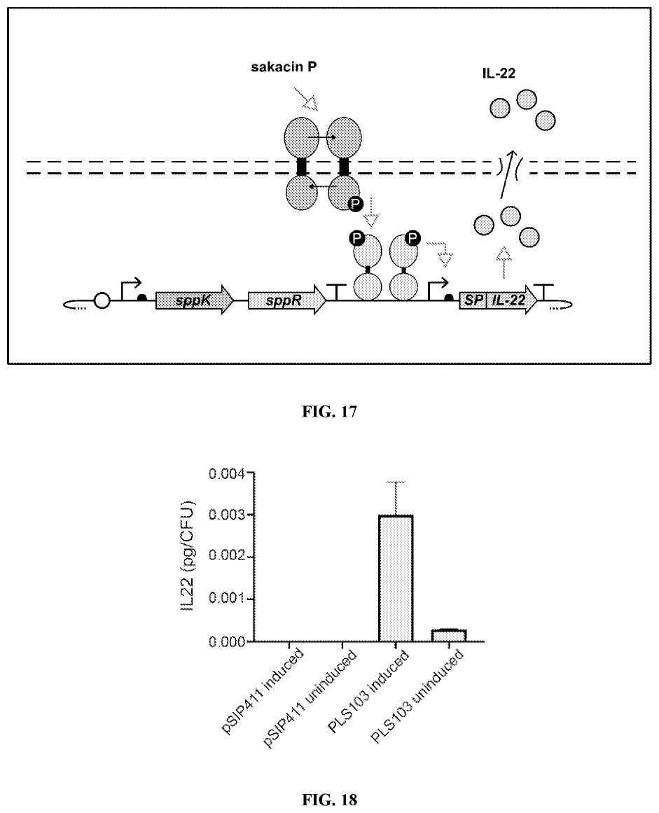

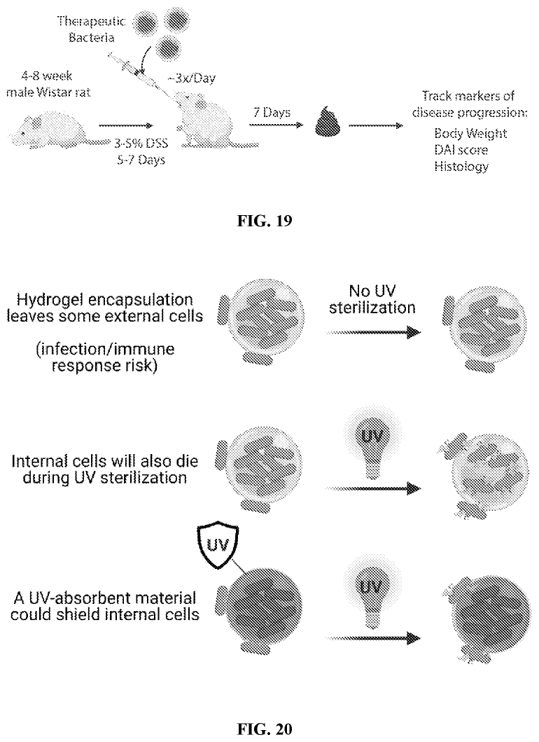

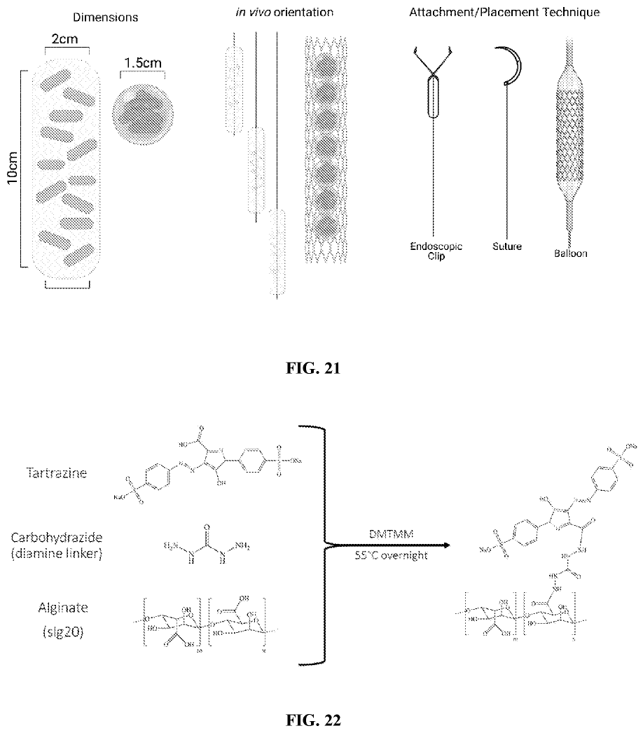

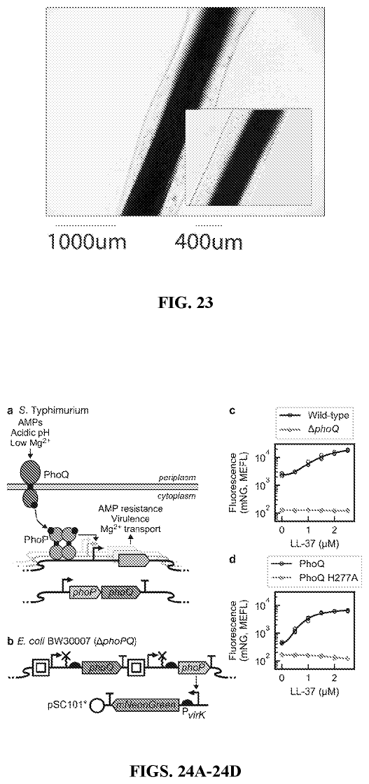

### DESCRIPTION OF ILLUSTRATIVE EMBODIMENTS

In certain embodiments, the present disclosure provides engineered bacteria, such as engineered bacteria that may be diagnostic, therapeutic, and/or theranostic. In some aspects, the engineered bacteria are engineered bacterial biosensors that respond to biomarkers of intestinal inflammation. The engineered bacteria may be encapsulated in a hydrogel matrix.

The matrix may be semi-permeable, allowing molecules under a certain size limit to be exchanged between the inner zone and the outer environment. The engineered bacteria may serve a dual role in sensing/reporting inflammation and secreting therapeutic proteins or small molecules in response to inflammation for disease treatment or management. Further embodiments provide methods for the use of the present engineered bacteria for the diagnosis or treatment of various diseases, such as inflammatory bowel disease (e.g., Crohn's or ulcerative colitis) or various cancers. For example, the present compositions may be used as a non-invasive tool for diagnosing and treating inflammatory bowel disease (IBD) and monitoring recurrences of inflammation. Further provided herein is the use of a lactic acid bacteria platform for vaginal applications in the gut environment. Additionally, in order to localize the therapy/diagnostic, enable long term treatment, and enable retrieval of the therapy/diagnostic, the present device may be in a cylindrical hydrogel structure that can be attached to the intestines via suture, or endoscopic clip (FIG. 16).

In certain embodiments, the present engineered bacteria may be formulated as an ingestible, implantable, inhalable, or injectable device for diagnostic or therapeutic purposes. This device may comprise an inner zone containing the engineered bacteria, and a semi-permeable outer zone that allows for bidirectional, diffusion-enabled exchange of molecules between the inner zone and the environment.

In some aspects, the present engineered bacterial biosensors respond to kynurenine, thiosulfate, oxidative stress, and other biomarkers of colonic inflammation. These bacteria can produce either a reporter molecule (e.g., a fluorescent protein reporter), and/or secrete a therapeutic molecule (e.g., an anti-inflammatory therapeutic molecule), in response to biomarker detection. The present biosensors may be engineered to secrete therapeutic proteins and/or non-protein molecules for disease treatment and management. In the present studies, the diagnostic potential of the thiosulfate sensor was demonstrated using the dextran sodium sulfate (DSS) rat model of colitis.

In some embodiments, the engineered bacterial biosensor comprises a two-component system (TCS) to sense pathogenic states. Targets for biosensing applications include, but are not limited to, pH, cadaverine, putrescine, nitrite, sialic acid, cellulose, estradiol, testosterone, androstenedione, and human antimicrobial peptides. The inventors previously developed a library of TCSs that were identified from the genomes of organisms in the human gastrointestinal tract. Alternatively, a library of TCSs identified from the genomes of organisms in the urogenital tract may be used. These libraries library may be screened for TCSs that are activated by the aforementioned target(s) using previously described methods. Sensors may be optimized for use as biosensors of human pathology, such as by increasing the dynamic range of the sensor, tuning the threshold of activation for the sensor, and/or optimizing expression of the sensing module in the target bacteria. These sensors can be used for genetic circuits that induce or repress the expression of a secondary molecule with diagnostic or therapeutic function. Molecules with diagnostic or therapeutic function that may be expressed either constitutively or dynamically from encapsulated bacteria include, but are not limited to, IL-6, IL-10, IL-12, IL-22, IL-35, IL-2, IL-7, IL-15, anti-NGF, bacteriocins, viral or bacterial antigens, human allergens, human cytokines, monoclonal antibodies, nanobodies, bacterial quorum sensing molecules, DNA, RNA, and viral vectors containing DNA or RNA.

In particular aspects, the TCS is a PhoPQ TCS from Salmonella Typhimurium and related Gram-negative pathogens that can sense human antimicrobial peptides (AMPs) RK-31, KS-27, KS-30, TLN-58, LL-29, ALL-38, GNLY, CCL20, TCP, KR-20, CCL-19, hBD3, and NPY. Each of these AMPs is produced by the human innate immune system to fight bacterial infections. However, they also have numerous other biological roles (e.g., immunomodulation, mucosal immunity, wound healing, adaptive immunity, and nervous system regulation) and have been implicated as disease biomarkers (e.g., CCL20 is a human intestinal biomarker of inflammation). Thus, in some aspects, the present disclosure provides engineered alginate-encapsulated bacterial strains to express PhoPQ in order to sense and report or sense and treat intestinal inflammation and other pathological states in vivo.

In some aspects, the engineered bacteria matrix is paramagnetic. In specific aspects, the metal oxide is titanium oxide, iron oxide, or ferrite. In particular aspects, the metal-based agent is ferric ammonium citrate. In some aspects, the hydrogel matrix further comprises iron oxide (US20130302252A1, U.S. Pat. No. 9,784,730B2; both incorporated herein by reference in their entirety). In certain aspects, the hydrogel matrix further comprises an ultraviolet (UV) absorbent small molecule (U.S. Ser. No. 10/183,477B2, U.S. Pat. No. 9,315,669B2, U.S. Pat. No. 9,670,300B2, KR102011152B1, CN105017871B, JP6224277B2; all incorporated herein by reference in their entirety). In some aspects, the hydrogel matrix has a specific density of chemical modifications (US20210145759A1, U.S. Ser. No. 10/786,446B2, WO2021026484A1, U.S. Ser. No. 10/730,983B2; all incorporated herein by reference in their entirety). In certain aspects, the hydrogel matrix is formulated to promote cell-material interactions between the semi-permeable polymer and the engineered bacteria (KR101465596B1, US20060292690A1, U.S. Ser. No. 10/786,446B2; all incorporated herein by reference in their entirety).

Prior to delivery, the bacteria are encapsulated within a superparamagnetic and porous hydrogel matrix. This device offers improvements compared to current methods for delivery of living therapeutics, and also compared to current standards of care for applicable diseases. Current diagnostic tools for many gastrointestinal diseases rely on medical intervention such as endoscopy and colon biopsy. In contrast, the present compositions and methods use a biosensor-based approach to diagnosis, which aims to reduce or eliminate the need for invasive procedures. In some aspects, the maximum size of the particles may be small relative to the size of the GI tract. Thus, the present device may comprise larger particles, have multiple particles linked together within a bacterial network or biomaterial, or may be implanted within the target site for long-term delivery.

The present devices comprising the engineered bacteria can enable improved survival and functionality of engineered bacteria, biocontainment in vivo, non-invasive administration through oral delivery, non-invasive diagnosis through collection of stool samples, and targeted delivery of therapeutics to the gut and other environments. Thus, the present methods and compositions, compared to current invasive methods, are amenable to persistent, long-term disease monitoring and treatment. The present compositions can deliver encapsulated, genetically engineered, diagnostic or therapeutic bacteria to an animal. By contrast, others have delivered un-encapsulated engineered bacteria (e.g., oral administration of bacterial culture), or encapsulated non-engineered bacteria thought to impart a therapeutic benefit. The present compositions enable the design of organisms that precisely diagnose the presence of physiological biomarkers of specific diseases and release appropriate therapeutics targeted at the relevant disease mechanisms. The present encapsulated engineered bacteria can i) survive transit through the gut at much higher numbers, ii) form a colony that survives in the gut long term without dilution and dispersal due to normal gastrointestinal flow, iii) be more easily analyzed to diagnose a disease, iv) secrete higher local amounts of drug, and v) be delivered to precisely the desired location where the disease is manifested.

### I. ENCAPSULATED ENGINEERED BACTERIA

The present compositions comprise engineered bacteria, particularly bacterial biosensors. The engineered bacteria may activate gene expression directly, or may activate an engineered genetic circuit that activates gene expression. The engineered bacteria may produce therapeutic and/or reporter (i.e., diagnostic) molecules, such as by extracellular secretion, production of molecules that diffuses extracellularly, or partial or complete lysis that releases molecules extracellularly.

Molecules with diagnostic or therapeutic function may be expressed either constitutively or dynamically from encapsulated bacteria. The therapeutic or reporter agent may be an immune system modulator or activator, e.g., a cytokine including but are not limited to, IL-6, IL-10, IL-12, IL-22, and IL-35), anti-NGF, bacteriocins, viral or bacterial antigens, human allergens, human cytokines, monoclonal antibodies, nanobodies, bacterial quorum sensing molecules, DNA, RNA, and viral vectors containing DNA or RNA.

The engineered bacteria may consist of multiple strains capable of communicating with each other. For example, the strain may be one or more of Escherichia coli, Lactobacillus reuteri, Lactobacillus crispatus, Lactobacillus rhamnosus, Lactobacillus crispatus, Lactococcus lactis, Bacteroides thetaiotamicron, or Bacillus subtilis bacterium.

The engineered bacteria may be transduced or transfected with a nucleic acid (e.g., a vector) comprising an expression sequence of a therapeutic agent or diagnostic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

Further provided herein is a device comprising encapsulated engineered bacteria. The device may comprise a cell or a plurality of cells, such as engineered bacteria. In the case of a plurality of cells, the concentration and total cell number may be varied depending on a number of factors, such as cell type, implantation location, and expected lifetime of the device. In some aspects, the device comprises 10¹ to 10¹⁰ bacteria. In an embodiment, the total number of cells included in the device is greater than about 2, 4, 6, 8, 10, 20, 30, 40, 50, 75, 100, 200, 250, 500, 750, 1000, 1500, 2000, 5000, 10000, or more. In an embodiment, the total number of cells included in a device is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In an embodiment, the total number of cells included in a device is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in a device is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within the device may be determined, e.g., prior to and/or after incorporation into the device. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### A. Biosensor System

The engineered bacterial biosensor may comprise one-component cytoplasmic proteins that sense ligands in the cytoplasm, membrane-bound two-component proteins that sense ligands outside the cell and regulate gene expression by communicating with cytoplasmic proteins, or cytoplasmic RNA molecules that bind cytoplasmic ligands and regulate gene expression

A two-component regulatory system serves as a bacterial stimulus-response coupling mechanism to allow organisms to sense and respond to changes in many different environmental conditions. Such systems typically consist of a membrane-bound histidine kinase that senses a specific environmental stimulus and a corresponding response regulator that mediates the cellular response, mostly through differential expression of target genes.

Two-component signal transduction systems enable bacteria to sense, respond, and adapt to a wide range of environments, stressors, and growth conditions. Some bacteria can contain hundreds of two-component sensor systems. These pathways have been adapted to respond to a wide variety of stimuli, including nutrients, cellular redox state, changes in osmolarity, quorum signals, antibiotics, temperature, chemoattractants, pH and more. As an example, in Escherichia coli the EnvZ/OmpR osmoregulation system controls the differential expression of the outer membrane porin proteins OmpF and OmpC.

Two-component systems accomplish signal transduction through the phosphorylation of a response regulator (RR) by a sensor histidine kinase (SK). SKs are frequently homodimeric transmembrane proteins containing a histidine phosphotransfer domain and an ATP binding domain, but SKs can be cytoplasmic. RRs may consist only of a receiver domain, but usually are multi-domain proteins with a receiver domain and at least one effector or output domain, often involved in DNA binding.

Upon detecting a particular change in the extracellular environment, the SK performs an autophosphorylation reaction, transferring a phosphoryl group from adenosine triphosphate (ATP) to a specific histidine residue. The cognate response regulator (RR) then catalyzes the transfer of the phosphoryl group to an aspartate residue on the RR receiver domain. This typically triggers a conformational change that activates the RR effector domain, which in turn produces the cellular response to the signal. Frequently, the effector domain is a DNA binding domain that enables the RR to stimulate (or repress) transcription from one or more target promoters and expression of one or more target genes.

Many SKs are bifunctional and possess phosphatase activity against their cognate RRs, so that their signaling output reflects a balance between their kinase and phosphatase activities. Many phosphorylated RRs also auto-dephosphorylate. The relatively labile phosphor-aspartate can also be hydrolyzed non-enzymatically. The overall level of phosphorylation of the RR population in the cell ultimately controls the activity of the two-component system.

It is possible to identify two-component systems from bacterial genome sequences by computational methods, such as homology and/or domain searching. However, such systems typically have unknown inputs and outputs. In the case of two-component systems wherein the RR has a DNA binding domain, this means that a computationally identified two-component system frequently controls expression of unknown output genes. Because both key pieces of information are lacking, and because the microbes that contain them are often un-culturable and/or difficult to genetically manipulate in the laboratory, it is very difficult to identify the inputs that they sense.

In a particular embodiment, there is provided an engineered bacterial two-component system PhoPQ from Salmonella Typhimurium and related Gram-negative pathogens that senses the human antimicrobial peptides (AMPs) RK-31, KS-27, KS-30, TLN-58, LL-29, ALL-38, GNLY, CCL20, TCP, KR-20, CCL-19, hBD3, and NPY. Each of these AMPs is produced by the human innate immune system to fight bacterial infections. However, they also have numerous other biological roles (e.g., immunomodulation, mucosal immunity, wound healing, adaptive immunity, and nervous system regulation) and have been implicated as disease biomarkers (e.g., CCL20 is a human intestinal biomarker of inflammation). Thus, in some embodiments, the present disclosure provides an engineered alginate-encapsulated bacterial strain to express PhoPQ in order to sense and report or sense and treat intestinal inflammation and other pathological states in vivo.

### B. Bacterial Encapsulation

The present device may comprise a permeable, semi-permeable, or impermeable material to control the flow of solution in and out of the device, such as a hydrogel matrix. For example, the material may be permeable or semi-permeable to allow free passage of small molecules, such as nutrients and waste products, in and out of the construct. In addition, the material may be permeable or semi-permeable to allow the transport of an agent, out of the device. Exemplary materials include polymers, metals, ceramics, and combinations thereof.

In some aspects, the present device comprises a polysaccharide (e.g., alginate, cellulose, hyaluronic acid, or chitosan). In particular aspects, the present device comprises alginate. The present device may comprise a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about 80 kDa, from about 20 kDa to about 70 kDa, from about 22.5 kDa to about 60 kDa, or from about 25 kDa to about 50 kDa). The present device may comprise at least 0.5%, 1%, 2%, 3%, 4%, 5% 7.5%, 10%, 15%, 20%, 30%, 40%, 50%, 60%, 70%, 80% or more of a polymer, e.g., a polymer described herein.

Alginate is a naturally occurring polymer comprising 0-(1-4)-linked mannuronic acid and guluronic acid residues, and as a result of its high density of negatively charged carboxylates, may be cross-linked with certain cations to form a larger structure, such as a hydrogel. Alginate polymers described herein may have an average molecular weight from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about 80 kDa, from about 20 kDa to about 70 kDa, from about 22.5 kDa to about 60 kDa, or from about 25 kDa to about 50 kDa). The present device may comprise at least 0.5%, 1%, 2%, 3%, 4%, 5%, 7.5%, 10%, 15%, 20%, 30%, 40%, 50%, 60%, 70%, 80% or more of an alginate polymer, such as an ultrapure alginate (e.g., SLG20 alginate).

In some aspects, the present device comprises a metal or a metallic alloy. Exemplary metals or metallic alloys include titanium (e.g., nitinol, nickel titanium alloys, thermo-memory alloy materials), platinum, platinum group alloys, stainless steel, tantalum, palladium, zirconium, niobium, molybdenum, nickel-chrome, cobalt, tantalum, chromium molybdenum alloys, nickel-titanium alloys, and cobalt chromium alloys. The present device may comprise stainless steel grade. The device may comprise at least 0.5%, 1%, 2%, 3%, 4%, 5%, 7.5%, 10%, 15%, 20%, 30%, 40%, 50%, 60%, 70%, 80% or more of a metal or metallic alloy, e.g., a metal or metallic alloy described herein.

In some aspects, the present device comprises a ceramic. Exemplary ceramics include a carbide, nitride, silica, or oxide materials (e.g., titanium oxides, hafnium oxides, iridium oxides, chromium oxides, aluminum oxides, and zirconium oxides). The present device may comprise at least 0.5%, 1%, 2%, 3%, 4%, 5%, 7.5%, 10%, 15%, 20%, 30%, 40%, 50%, 60%, 70%, 80% or more of a ceramic, e.g., a ceramic described herein.

The present device may comprise glass. The device may comprise at least 0.5%, 1%, 2%, 3%, 4%, 5%, 7.5%, 10%, 15%, 20%, 30%, 40%, 50%, 60%, 70%, 80% or more glass.

A material within the present device may be further modified, for example, with a chemical modification. For example, a material may be coated or derivatized with a chemical modification that provides a specific feature, such as an immunomodulatory or antifibrotic feature. Exemplary chemical modifications include small molecules, peptides, proteins, nucleic acids, lipids, or oligosaccharides. The present device may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein.

In some embodiments, the material is chemically modified with a specific density of modifications. The specific density of chemical modifications may be described as the average number of attached chemical modifications per given area. For example, the density of a chemical modification on a material in, on, or within a device described herein may be 0.01, 0.1, 0.5, 1, 5, 10, 15, 20, 50, 75, 100, 200, 400, 500, 750, 1,000, 2,500, or 5,000 chemical modifications per square μm or square mm.

The chemical modification of a material may include a linker or other attachment moiety. These linkers may include a cross-linker, an amine-containing linker, an ester-containing linker, a photolabile linker, a peptide-containing linker, a disulfide-containing linker, an amide-containing linker, a phosphoryl-containing linker, or a combination thereof. A linker may be labile (e.g., hydrolysable). Exemplary linkers or other attachment moieties is summarized in Bioconjugate Techniques (3^{rd} ed, Greg T. Hermanson, Waltham, MA: Elsevier, Inc, 2013), which is incorporated herein by reference in its entirety.

Cell encapsulation within a hydrogel can leave a fraction of the cells on the hydrogel surface, which could cause complications after implant. Thus, in some aspects, the present device comprises a UV absorbent small molecule or nanoscale material. The UV absorbent small molecule may be a dye or photoblocker, such as tartrazine (Yellow 5). The UV absorbent nanoscale material may be a metallic crystal, synthetic polymer, natural polymer, or carbon-based material. In specific aspects, the UV absorbent molecule may be attached to alginate, which may be then crosslinked with barium cations.

In order to increase the mechanical properties of alginate, polyacrylamide, a secondary polymer that can form covalent bonds with itself and alginate (Tang et al., 2021) may be used. These combination materials have greatly increased durability and resistance to fracturing while also retaining their biocompatibility and permeability to small molecules, allowing for viable cell encapsulation. Polyacrylamide-alginate can be used to coat alginate hydrogels, allowing for specific architectures like microcapsules or strings to be made first and subsequently reinforced via this tough coating. This method makes use of an aqueous pregel solution: alginate, acrylamide, ammonium persulfate, N,N-methylenebisacrylamide (MBAA, crosslinker), and N,N,N′,N′-tetramethylethylenediamine (TEMED, an accelerator). The hydrogels being coated can be dipped in this pregel solution, under nitrogen atmosphere, and can then be transferred to MES buffer containing 1-ethyl-3-(3-dimethylaminopropyl)carbodiimide, N-hydroxysuccinimide, and adipic acid dihydrazide to covalently bond the polyacrylamide-alginate coating.

Polyacrylamide-alginate hydrogels can also be made directly by combining a similar aqueous pregel solution with an alginate-crosslinking solution of calcium sulfate, followed by curing with UV light. Cells may be loaded into these gels after gel formation, and this method can allow for molding a wide variety of architectures.

Other methods for improving mechanical properties that may be used include but are not limited to increasing the alginate molecular weight or increasing crosslinker density (e.g., letting the hydrogel crosslink for longer). Both of these methods can result in uniform changes throughout the hydrogel, with the former making the hydrogel more interconnected and the latter focusing on more tightly binding individual alginate polymers together. Another method may comprise incorporating another polymer, such as PEG or chitosan, to act as structural reinforcement. Additionally, UV-based covalent crosslinking methods may be added to the system during the same step without endangering the encapsulated cells.

In some aspects, the device may comprise a degradable component. “Degradable,” as used herein, refers to a structure which upon modulation, e.g., cleavage, decreases the ability of the zone of the device (e.g., the inner zone and/or the outer zone) to impede contact of a host immune effector with the zone (e.g., the inner zone and/or the outer zone) or a component disposed in the zone. For example, the degradable entity can comprise a site which is cleavable by an enzyme, e.g., an endogenous host enzyme, or an administered enzyme. Typically, the degradable entity mediates a physical property of a zone, e.g., the inner zone or the outer zone, for example, the thickness, degree of cross-linking, or permeability, which impedes passage of a host agent (e.g., a host immune component, e.g., a host immune cell).

### C. Therapeutic Agents

A device described herein may contain a therapeutic agent, for example, produced or secreted by a cell. A therapeutic agent may include a nucleic acid (e.g., an RNA, a DNA, or an oligonucleotide), a protein (e.g., an antibody, enzyme, cytokine, hormone, receptor), a lipid, a small molecule, a metabolic agent, an oligosaccharide, a peptide, an amino acid, an antigen. For example, the device comprises bacteria that are genetically engineered to produce or secrete a therapeutic agent.

The engineered bacteria may be engineered to produce or secrete a protein. The protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may be include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, blood clotting factor (e.g., Factor VII, Factor VIII, Factor IX, GCG, or VWF), hemoglobin, enzymes, proenkephalin, a growth factor (e.g., EGF, IGF-1, VEGF alpha, HGF, TGF beta, bFGF), or a cytokine.

A protein produced or secreted by a cell may include a hormone. Exemplary hormones include growth hormone, growth hormone releasing hormone, prolactin, lutenizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

A protein produced or secreted by a cell may include a cytokine. A cytokine may be a pro-inflammatory cytokine or an anti-inflammatory cytokine. Example of cytokines include IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M. J. Cameron and D. J. Kelvin, Cytokines, Chemokines, and Their Receptors (2013), Landes Biosciences, which is incorporated herein by reference in its entirety.

The present device may comprise a cell expressing a single type of therapeutic agent, e.g., a single protein or nucleic acid, or may express more than one type of therapeutic agent, e.g., a plurality of proteins or nucleic acids. The engineered bacteria may express two, three, or four types of therapeutic agents (e.g., two types of proteins or nucleic acids) or three types of therapeutic agents (e.g., three types of proteins or nucleic acids).

### D. Device Formulation and Administration

The device described herein may take any suitable shape or morphology. For example, the present device may be a sphere, spheroid, tube, cord, string, ellipsoid, disk, cylinder, sheet, torus, cube, stadiumoid, cone, pyramid, triangle, rectangle, square, or rod. The device may comprise a curved or flat section. The device may be prepared through the use of a mold, resulting in a custom shape. In specific embodiments, the device is in a sphere confirmation or cylindrical noodle confirmation.

The device may vary in size, depending, for example, on the use or site of implantation. For example, the device may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. The device may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. The device may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. The device may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

The device may comprise at least one zone capable of preventing exposure of an enclosed antigenic or therapeutic agent from the outside milieu, e.g., a host effector cell or tissue. The device may comprise an inner zone (IZ) and an outer zone (OZ). Either the inner zone (IZ) or outer zone (OZ) may be erodible or degradable.

The zone (e.g., the inner zone or outer zone) of the device may comprise a degradable entity, e.g., an entity capable of degradation. A degradable entity may comprise an enzyme cleavage site, a photolabile site, a pH-sensitive site, or other labile region that can be eroded or comprised overtime. The degradable entity may be degraded upon exposure to a first condition (e.g., exposure to a first milieu, e.g., a first pH or first enzyme) relative to a second condition (e.g., exposure to a second milieu, e.g., a second pH or second enzyme). In some aspects, the degradable entity is degraded at least 2, 5, 10, 20, 30, 40, 50, 60, 70, 80, or 100 times faster upon exposure to a first condition relative to a second condition. The degradable entity may be an enzyme cleavage site, e.g., a proteolytic site, a polymer (e.g., a synthetic polymer or a naturally occurring polymer, e.g., a peptide or polysaccharide), a substrate for an endogenous host component, e.g., a degradative enzyme, e.g., a remodeling enzyme, e.g., a collagenase or metalloprotease. The degradable entity may comprise a cleavable linker or cleavable segment embedded in a polymer.

The device may comprise pores to permit passage of an object, such as a small molecule (e.g., nutrients or waste), a protein, or a nucleic acid. For example, pores may be greater than 0.1 nm and less than 10 μm. In some aspects, the device comprises pores with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

A device described herein may comprise a chemical modification in or on any enclosed material. Exemplary chemical modifications include small molecules, peptides, proteins, nucleic acids, lipids, or oligosaccharides. The device may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. The device may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In some aspects, the present device is formulated such that the duration of release of the therapeutic and/or diagnostic agent is tunable. For example, the device may be configured in a certain manner to release a specific amount of a therapeutic and/or diagnostic agent over time, e.g., in a sustained or controlled manner. The device comprises a zone (e.g., an inner zone or an outer zone) that is degradable, and this controls the duration of therapeutic release from the construct by gradually ceasing immunoprotection of encapsulated cells or causing gradual release of the agent.

In some embodiments, the device is chemically modified with a specific density of modifications. The specific density of chemical modifications may be described as the average number of attached chemical modifications per given area. For example, the density of a chemical modification on or in the present device may be 0.01, 0.1, 0.5, 1, 5, 10, 15, 20, 50, 75, 100, 200, 400, 500, 750, 1,000, 2,500, or 5,000 chemical modifications per square μm or square mm.

The device may be formulated or configured for implantation in any organ, tissue, cell, or part of a subject. For example, the device may be implanted or disposed into the intraperitoneal space of a subject. The device may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. The device may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

Multiple devices may be configured in a “beads in a mesh” design which will fix/attach the devices to the target tissue. This design consists of the devices enclosed in a noddle-shaped mash fixed to the target tissue via a surgical thread (21). Nutrients, proteins, and other relevant molecules can pass freely through the mesh and diffuse into or out from the devices OZ and IZ. Most of the mesh surface area may be holes/gaps that are smaller than the devices to prevent the escape of devices from the mesh. In addition, to prevent the release of the devices from the mash, it may be closed on both sides using welding, knots, or other methods. The device may be attached to its target site via surgical thread fix to the treated or/and diagnosed tissue. The mesh may be made from alloy or other biocompatible polymers. The devices enclosed in the mash can be fixed to the target tissue via a suture, an endoscopic clip and/or a balloon (21) connected to the mesh.

The present device may be formulated for use for any period of time. For example, the present device may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. The present device can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). The present device can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). The present device can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

The present disclosure provides pharmaceutical compositions comprising encapsulated engineered bacteria. Such compositions comprise a prophylactically or therapeutically effective amount of the encapsulated engineered bacteria, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

### E. Methods of Treatment

In particular, the present compositions that may be used in treating a disease in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (Complete blood count (CBC)), or cancer cell growth or proliferation.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD 117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker either in healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### II. KITS

In various aspects of the embodiments, a kit is envisioned containing therapeutic agents and/or other therapeutic and delivery agents. In some embodiments, the present embodiments contemplates a kit for preparing and/or administering a device of the embodiments. The kit may comprise one or more sealed vials containing any of the pharmaceutical compositions of the present embodiments. The kit may include, for example, engineered bacteria as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may comprise one or more reagents for a biotechnology product or assay. The kit may further comprise regents for in vitro assays such as western blots, flow cytometry, immunoprecipitation, ELISA, or immunofluorescence.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Encapsulated Engineered Bacteria

Bacterial encapsulation. Alginate hydrogel spheres were synthesized using a custom-built, two-fluid co-axial electrostatic spraying device. The device consisted of a voltage generator that was attached to the tip of a co-axial needle and grounded to a 1:4 barium chloride:mannitol crosslinking bath. The co-axial needle was fed by two separate syringes containing 1.4% alginate solutions that were diluted in 0.8% saline. The alginate used for the core contained engineered bacteria. The bacterial culture was diluted 1:100 in fresh LB medium (3 μL in 3 mL), incubated at 37° C. and shaken to OD₆₀₀=1. 1 mL of the culture was centrifuge (5000 g, 4 min) and washed with sterile PBS then centrifuged again. The cell pellet was resuspended with 50 μL sterile PBS (to make the mixing with the alginate easier) and then mixed with alginate at a volume that would dilute the cells to the desired OD. The desired volume of SLG20 alginate was added to the pellet and pulled up by a 5 ml luerlock syringe. Syringes containing the two different alginates were placed in two separate syringe pumps that were suspended over 150 ml of the crosslinking bath. Flow rates for each syringe were 5 ml/hr and 14 ml/hr for the core and shell respectively. Size of the capsules was maintained by adjusting the voltage on the generator; a voltage of ˜5.60 most consistently produced capsules that were 1.5 mm in diameter. Capsules were decanted in order to separate them from the crosslinking bath. They were subsequently washed 3 times with HEPES buffer.

Bacterial noodle encapsulation. Hydrogel noodles were made using a custom-built, two-fluid co-axial electrostatic spraying device. A 1:4 barium chloride: mannitol crosslinking bath was used to gel the alginate. A co-axial needle was fed by two separate syringes containing alginate dissolved at a 1.4% wt/v concentration in 0.8% saline. E. coli bacteria at OD₆₀₀: 1 resuspended in 50 μL sterile PBS was mixed with 1 ml of the dissolved alginate and taken up by a 5 mL syringe. The syringe feeding the shell (also in a 5 mL syringe) was free of bacteria. Both syringes were attached to the co-axial needle which was submerged into the crosslinking bath. The flow rates for the core and shell syringe pumps were set to 15 mL/h. The alginate was expelled until none remained in the syringes. The noodle incubated in the bath for 15 minutes and was subsequently washed 3× with HEPES buffer. The noodle was then sliced into 1-inch sections and placed into 2 mL Eppendorf tubes where 500 μl of EDTA was added. The noodle was homogenized using an electric homogenizer. The free bacteria were diluted in pbs and plated on agar plates in order to count CFU/noodle section. The plates were incubated at 37° C. and colonies were counted 24 hours later.

Preparation of iron oxide nanoparticle-alginate mixture. Carboxylic acid-functionalized, 20 nm iron oxide nanoparticles (Ocean Nanotech, SHP-20) at the stock concentration of 5 mg/mL were put in dialysis against MilliQ water to remove the 0.02% sodium azide in the stock solution. The dialysis tubing used (Sigma, D2272) was benzoylated and had a MWCO of 2000 Da. The water was changed after the first two hours, and the nanoparticle solution was left in dialysis overnight. The nanoparticle solution was removed from the tubes the following day, the recovered volume was recorded, and the nanoparticle solution was diluted as necessary (e.g., to 1.8 mg/mL). Sodium chloride (Sigma, S7653, BioXtra) was dissolved in the nanoparticle solution to achieve a final concentration of 0.8 wt. %. Sodium alginate (Pronova, UPLVG 1703) was dissolved in the nanoparticle solution overnight to achieve a final concentration of 1.4 wt. %

Screening of TiO₂ nanoparticles for UV absorbance. Ten different titanium oxide nanoparticles were suspended at 1 wt. % in 1.4 wt. % alginate (Pronova SLG20) with two hours in a heated sonication bath. The TiO₂-alginate was diluted to 0.05 wt. % TiO₂ in alginate with additional alginate solution, mixed by vortexing, and n=3 wells of a quartz-bottom 96-well plate were filled with 75 μL of the diluted TiO₂-alginate solution. The process of diluting, mixing, and plating was repeated to get 0.01 wt. % and 0.005 wt. % of each TiO₂ nanoparticle in alginate. Once all three concentrations for a single nanoparticle type were made and plated, the plate was quickly put through an absorbance scan at 365 nm light in a Tecan plate reader. To prevent TiO₂ from falling out of suspension, only one TiO₂ nanoparticle type was plated and scanned at a time as opposed to preparing all solutions before plating. Additionally, the above process was repeated to obtain absorbance spectrums for the ten TiO₂ nanoparticles in alginate for 230-400 nm. TiO₂ nanoparticles used included U.S. Research Nanomaterials nanopowder stocks US3501 (5 nm amorphous), US3492 (15 nm anatase), US3498 (30 nm anatase), US3411 (100 nm anatase), US3520 (30 nm rutile), US3530 (50 nm rutile), US3535 (100 nm rutile), and Sigma-Aldrich 718467 (21 nm titanium dioxide “Aeroxide”) and 791326 (<100 nm brookite).

General preparation of titanium oxide nanoparticle-alginate mixture. Titanium oxide nanoparticles (e.g., 30 nm rutile TiO₂ from U.S. Research Nanomaterials, Inc.) were suspended in water or a pre-dissolved alginate solution at 1 wt. % TiO₂ over two hours in a heated sonication bath. Prior to use with cells or being made into a hydrogel construct, the TiO₂-alginate solution was filtered with a 5 μm syringe filter. If the solution includes another component that cannot be further diluted, e.g., a premade suspension of iron nanoparticles, TiO₂ nanoparticle suspension is the last step since it does not require changing the alginate concentration.

Preparation of UV-absorbent tartrazine-alginate. Sodium alginate (Pronova SLG20) was dissolved at 1 wt % in MilliQ water. All molar equivalency calculations are based on the 5.1 mmol —COOH per gram of alginate that will be used for functionalization. Additionally, the solution was kept in the dark for the duration of the reaction since tartrazine is light sensitive. One molar equivalent of carbohydrazide (Sigma) was added to the solution, followed by 1 molar equivalent of tartrazine (Sigma). Once these reagents were dissolved, 0.75 molar equivalent of DMTMM was dissolved in 50 mL of MilliQ water and added dropwise to the reaction solution. The reaction was kept in the dark at 55° C. overnight. The solution was placed in 12 kDa pore size dialysis tubing the following day against MilliQ water, which was changed twice daily for a week.

Bacterial engineering methods. Bacterial strains were genetically engineered using conventional genomic and plasmid-based approaches, such as polymerase chain reaction and golden gate assembly. Molecular biology methods are described further in Daeffler et al. (2017).

Bacterial Viability and Sterilization Experiments. Bacteria were encapsulated as described previously. Samples were plated before encapsulation for each of the sample groups in order to obtain a CFU count. Capsules were exposed to various intensities of UV radiation for various durations of time, using a unidirectional UV source at 365 nm. One control group of capsules was left unexposed, to assess bacterial viability after encapsulation. Capsules were transferred to sterile tubes and the buffer was aspirated. Each tube contained 5 capsules, to which 500 μL sterile EDTA (pH 8) was added. Capsules were homogenized with an electric homogenizer blade at speed 3 for 10-20 seconds until there are no visible capsule fragments remaining. In between samples, the homogenizer blade was cleaned by running it at high speed, first in sterile DI H₂O, then dried, then sterilized with EtOH for 5 sec, then washed with sterile PBS and dried again. Solutions were placed on ice for downstream analysis or diluted and plated immediately to obtain CFU counts as a measure of bacterial survival. Plates were incubated overnight at 37° C.

For sterilization experiments, the capability to sterilize the capsule surface was demonstrated by incubating bacterially void capsules in a bacterial culture containing the maximum amount of bacteria possible to contaminate the buffer during the encapsulation process. The ability to sterilize the buffer alone was also demonstrated by incubating and plating the buffer from capsules containing the regular amount of bacteria. To demonstrate internal bacterial viability, the homogenized capsules were plated and their numbers compared to those present in the equivalent volume of both unencapsulated bacteria and unsterilized capsules.

Biosensor response assays. After overnight bacterial growth, preparation, and encapsulation, capsules were washed 3 times in sterile PBS. In a 96-well plate, 5 capsules were aliquoted per well. The native buffer was aspirated and replaced with 200 μL/well of sterile M9 media (% glycerol) containing varying concentrations of the biomarker being assayed (i.e., thiosulfate, kynurenine, Mg²⁺, pH, or peptide). For unencapsulated controls, samples were diluted as if into alginate (50 μL of cells from 10 mL culture at OD 0.1, added to 950 μL of alginate), and the same amount of bacteria was placed in each well as was present inside 1 capsule (ex. if 5 capsules/well: core volume of 1 capsule is 0.9 μL of alginate-bacterial mix, so bacterial volume of 5 capsules ˜ 4.5 μL). Samples were incubated for 4.5 hr at 37° C. stationary. Capsules were washed 3 times in sterile PBS and homogenized using standard protocol detailed previously. If nanoparticles were included in the capsule formulation, the homogenized solution was be placed on 96 well magnetic block, on ice for 20 min to draw nanoparticles to the bottom of the wells (to decrease the noise in the flow cytometer). The supernatant was then removed without disturbing the nanoparticles and transferred to flow cytometry tubes. The homogenized sample was then diluted into 1 mL sterile PBS (add 50 μL homogenized sample to 950 μL PBS→a 10× dilution) and transferred to a flow cytometry tube. Samples were run through the flow cytometer using the FL3 channels, to gate only the bacteria and not the nanoparticles and alginate debris. Data was collected to 10K counts on the flow with settings FL1=600 FL2=476 FL3=563 BCD 2, 9, 14.

IL-22 quantification. Capsules containing engineered L. reuteri were made as described above and incubated with sakacin P. inducing peptide at 37° C. for 6 hours at which point the supernatant was collected and frozen. The concentration of IL-22 was determined via ELISA.

Diagnosis using DSS model of intestinal inflammation and gavage. All animal studies were performed in accordance with guidelines established by the Animal Welfare Committee. Sprague dawley rats were weighed on day 0, prior to any manipulation. Stool was also collected and checked for occult blood as a negative control. To establish intestinal inflammation, rats were provided drinking water with 3% wt/v dextran sulfate sodium for 7 days. Rats were monitored daily for body weight and presence of occult blood in the stool. A disease activity index score was used for the comparative analysis of intestinal bleeding; no weight loss, normal stool consistency, negative hemoccult: 0; 1-5% weight loss, positive hemoccult but no visible blood, loose/soft stools: 1; 5-10% weight loss, positive hemoccult with visual pellet bleeding, Very soft stools: 2; 10-15% weight loss, blood around the rectum/prolapse, watery stools: 3. 15-20% weight loss, gross bleeding, and diarrhea:4. After 7 days, scores were recorded and rats were dosed with 1.5 mL of encapsulated bacteria in 5 ml of PBS containing 10% wt/v sodium bicarbonate via oral gavage. 6 hours post gavage, rats were separated and fecal samples were collected and capsules were retrieved, transferred to Eppendorf tubes with PBS and kept on ice. 5 capsules from 3 separate fecal samples were used for measurement of sfGFP reporter via flow. Prior to homogenization, the PBS was aspirated and 500 ul of 50 mM EDTA was added to each repeat. Capsules were homogenized using an electric homogenizer at speed 3 for 10-20 seconds until no capsule fragments were visible. The homogenized solution was placed immediately on ice for processing.

### Example 2—Cylindrical Alginate

The alginate cylindrical noodle was formed by resuspending a 50 ul bacterial pellet in 950 ul alginate, mixing with a pipette, loading a 5 ml BD plastic syringe with bacteria (core), loading a 5 ml BD plastic syringe with alginate only (shell), setting the syringe pump to 15 ml/hour for both the core and shell, filling the crosslinking dish with ˜150 ml crosslinking buffer, submerging the co-axial needle into the bath, washing 3× with HEPES, and keeping bacteria in Ca+KREBS.

SLG20 alginate, used for the core of the core-shell hydrogels, was purchased from PRO NOVA. It was dissolved in 0.8% saline at 1.4% weight per volume. Following alginate and cell preparation, hydrogels were synthesized using a two fluid co-axial electrostatic spraying device which consisted of two syringe pumps. Each of the pumps held a syringe, one containing SLG20 alginate with the cell suspension, and the other containing SLG20 alginate alone. The syringes were connected using a co-axial needle that was submerged in a 1:4 barium chloride: mannitol crosslinking bath. The bath was supplemented with 0.65% Tween20®. The pumps were set to run at 15 ml per hour. Noodle was incubated in the crosslinking bath for 15 minutes and subsequently washed three times with HEPES buffer, followed by three times with complete media.

To incorporate the thread, it was fed feed the syringe in a spiral. The needle was attached and the thread was pulled through to stick out. When the alginate came out of the syringe it pushed the thread out with it. The alginate was allowed to crosslink as it regularly does. Once it is fully crosslinked the alginate was shaved off to have coated sections of the desired length. With the early, non-coated sections of thread, a suture or a clip was tied for the desired attachment technique.

To produce the tartrazine-alginate (FIG. 15) to protect the bacteria inside and allow for sterilization of the outside with UV light, alginate (NOVAMATRIX SLG20) was dissolved in MilliQ water at 2 wt. %. 0.75 equivalent of tartrazine powder (Sigma) and 0.5 equivalent of carbohydrazide (Sigma), as a diamine linker, were added to the solution. 0.5 equivalent of DMTMM was dissolved in 20 mL of MilliQ water and was added dropwise. The reaction was left in the dark at 55° C. overnight. The following day, the solution was put in dialysis against MilliQ water for 5 days before lyophilization.

To characterize the material's UV absorbance, solutions with various concentrations of tartrazine-alginate were prepared by mixing solutions of tartrazine-alginate and unmodified slg20 alginate (both dissolved at 1.4 wt. % in 0.8% saline). Specifically, the solutions made were 0, 25, 50, 75, and 100% tartrazine-alginate. These solutions were then plated into a quartz-bottom 96 well plate and scanned for UV absorbance with a Tecan plate reader (FIG. 20). Two alternative UV wavelengths are shown on the plot.

Tartrazine-alginate was crosslinked with divalent cations as the unmodified alginate. Specifically, a 1.4 wt. % solution of tartrazine-alginate in 0.8% saline was made and dropped into a crosslinking bath (20 mM barium chloride, 5 wt. % mannitol, 0.01 vol. % Tween 20) to form capsules (FIG. 18). The capsules shown were made using only a syringe, but electrospraying can be used to control the capsule diameter. The tartrazine-alginate retained the yellow color of the dye used to make it.

### Example 3—High-Throughput Discovery of Peptide Activators of a Bacterial Sensor Kinase

Bacteria use two-component system (TCS) signaling pathways to sense and respond to peptides involved in host defense, quorum sensing, and inter-bacterial warfare. However, little is known about the peptide-sensing capabilities of these TCSs. A high-throughput E. coli display method was developed to characterize the effects of human antimicrobial peptides (AMPs) on the pathogenesis-regulating TCS PhoPQ of Salmonella Typhimurium. IT was found that PhoPQ senses AMPs comprising diverse sequences, structures, and biological functions. Using thousands of AMP variants, sub-domains and biophysical features responsible for PhoPQ activation were identified. It was shows that most of the newfound activators induce PhoPQ in S. Typhimurium, suggesting a role in virulence regulation. Finally, it was found that PhoPQ homologs from Klebsiella pneumoniae and extraintestinal pathogenic E. coli, which occupy different in vivo niches, exhibit distinct AMP response profiles. The high-throughput method enables new insights into the specificities, mechanisms, and evolutionary dynamics of TCS-mediated peptide sensing in bacteria.

PhoPQ is a peptide-sensing TCS that regulates virulence in Gram-negative enterobacterial pathogens. This TCS is best studied in Salmonella Typhimurium. Typically S. Typhimurium bacteria enter the body through ingestion of contaminated food or water. Once inside, they infect intestinal epithelial cells, cross the epithelial barrier, and are endocytosed by immune cells, including macrophages, where they survive and replicate intracellularly in a Salmonella-containing vacuole. Here, the membrane-bound sensor histidine kinase PhoQ detects antimicrobial peptide (AMPs) produced as part of the human innate immune response, as well as acidic pH. PhoQ can also sense low divalent cation concentrations, though this stimulus is likely less important in vivo. When activated, PhoQ phosphorylates the response regulator PhoP. Phosphorylated PhoP binds to genomic target promoters and directly or indirectly modulates transcription of more than 100 genes, including those involved in AMP resistance and virulence pathways that are essential for infection.

Despite decades of investigation, little is known about the AMP-sensing capabilities of PhoQ. Previous studies of peptide-TCS interactions have largely relied on chemical peptide synthesis, which is costly and limited to short, linear peptides in high-throughput applications. By contrast, host AMPs often contain disulfide bonds and can extend to tens of amino acids in length. Due to these limitations, studies of AMP-PhoQ interactions have examined fewer than nine peptides each, most of which shared similar biochemical and biophysical properties. These studies suggest that cationic AMPs bind to an acidic patch within the PhoQ periplasmic domain, and that the extent of positive net charge and hydrophobicity are positively correlated with PhoQ activation. Most of the 16 known PhoQ-activating AMPs, including the only known human activator cathelicidin LL-37 and its mouse ortholog cathelicidin-related antimicrobial peptide (CRAMP), are cationic and amphipathic.

In an effort to develop novel antimicrobial peptides, surface-localized antimicrobial display (SLAY), a multiplexed screening approach for determining the impact of peptides on bacterial viability may be used (Tucker et al., 2018). In SLAY, E. coli are engineered to express a fusion protein that tethers peptides to the outer membrane (OM). The tether is of sufficient length that displayed peptides can penetrate the OM and interact with the periplasm and inner membrane (IM), yet short enough that they can only interact with the cell from which they are expressed. This cis activity enables peptide sequence to be directly linked to phenotype (i.e. growth) via next generation DNA sequencing (NGS). SLAY also permits the formation of disulfide bonds to enable surface display of AMPs with more complex structures, such as defensin HNP-1. Despite its demonstrated utility for AMP development, SLAY has not been utilized to characterize peptide-TCS interactions.

In the present studies, SLAY was combined with heterologous TCS expression and sort-seq, a high-throughput fluorescent reporter gene expression assay based on fluorescence-activated cell sorting (FACS) with NGS, to measure the response of S. Typhimurium PhoPQ to 117 human AMPs and thousands of variants of those AMPs in laboratory E. coli. This method is referred to as SLAY-TCS. Using SLAY-TCS, 13 human AMP activators of PhoQ were identified, the effects of subdomains of human AMP activators with combined α+β structures on PhoQ were evaluated, and peptide biochemical and biophysical properties associated with PhoQ activation were identified. These newfound activators were validated using chemical and recombinant peptide synthesis and PhoPQ assays in S. Typhimurium. Finally, a simplified SLAY-TCS protocol was used wherein sort-seq is replaced with flow cytometry to discover that PhoPQ orthologs from extraintestinal pathogenic E. coli (ExPEC) and Klebsiella pneumoniae have evolved altered AMP sensing specificities relative to S. Typhimurium PhoPQ. It was demonstrated that PhoQ senses select human AMPs in a specific manner. The results suggest a role for evolutionary adaptation in AMP sensing specificity and provide the first method for high-throughput characterization of peptide-TCS interactions.

Porting S. Typhimurium PhoPQ into E. coli: To enable high-throughput screening of AMP-PhoQ interactions, an E. coli reporter strain of S. Typhimurium PhoPQ activity was engineered (24). First, a genomic integration cassette was constructed wherein S. Typhimurium phoP and phoQ are expressed from constitutive promoters and other well-characterized gene regulatory elements (24). The resulting engineered PhoPQ expression cassette was integrated into the genome of E. coli BW30007, which lacks the E. coli phoPQ ortholog, resulting in strain KB1. Next, a plasmid-based transcriptional reporter of PhoPQ activity was developed. When overexpressed, PhoP can activate target promoters independently of PhoQ. Thus, five plasmids were constructed, each containing an anhydrotetracycline (aTc)-inducible phoP and a different S. Typhimurium PhoP-activated promoter driving expression of a superfolder GFP (sfgfp) fluorescent reporter gene. These plasmids were transformed into BW30007 and measured sfGFP fluorescence in response to varying levels of aTc. It was found that the virK promoter (P_{virK}) yields the largest fold activation in response to aTc exposure. Thus, P_{virK} was selected as a reporter of PhoPQ activity for the subsequent experiments.

Next, a plasmid was constructed wherein P_{virK} controls expression of an mNeonGreen (mNG) fluorescent reporter gene, which produces a fluorescent protein that is brighter than sfGFP. This plasmid was transformed into both S. Typhimurium and KB1 to measure PhoPQ activation by the positive control AMP LL-37. It observed that this P_{virK}-mNG reporter system is activated in a similar manner in both organisms (24), with half-maximal activation occurring at 2.01±0.43 and 1.16±0.10 μM LL-37, respectively. LL-37 response was also measured in two negative control strains: an S. Typhimurium strain lacking phoQ and a KB1 derivative in which the catalytic histidine of PhoQ was mutated to an alanine (KB1 PhoQ H277A). Neither negative control strain responds to LL-37, demonstrating that S. Typhimurium PhoPQ specifically senses this AMP in both its native context and in E. coli KB1. These results demonstrate that the function of S. Typhimurium PhoPQ was recapitulated in E. coli strain KB1.

PhoPQ senses surface-displayed peptides in E. coli: Next, it was validated that PhoPQ can detect AMPs displayed on the surface of KB1. In SLAY, peptides of interest are expressed at the C-terminus of a fusion protein consisting of the Lpp signal peptide, an OmpA transmembrane domain, and a flexible tether. Here, a series of 13 displayed peptide expression plasmids were constructed adapted from the P_{virK}-mNG reporter plasmid. In addition to P_{virK}-mNG, each of these plasmids encodes a SLAY fusion protein under the control of an isopropyl-β-D-thiogalactoside (IPTG)-inducible promoter (P_{tac}) (FIG. 25). P_{tac} is tightly repressed, to avoid potential toxicity arising from leaky AMP expression in the absence of inducer. Using this platform, the PhoPQ response was measured to nine positive control peptides that had been shown to activate PhoPQ in previous studies (FIG. 25). Cecropin P1 was included, which had previously been displayed with SLAY, as a tenth putative activator, as a phoQ mutant strain had previously exhibited increased susceptibility to this peptide (FIG. 25). Two negative control peptides were also measured: human defensin HNP-1, which had previously been shown not to activate PhoPQ, and the scrambled, inert peptide NC, which had previously been shown to be non-toxic to S. Typhimurium phoP and phoQ mutant strains. Finally, a no-peptide control plasmid encoding a stop codon was constructed instead of a peptide following the OmpA transmembrane domain. This plasmid enabled quantification of the baseline level of fluorescence from the P_{virK}-mNG reporter in the absence of any displayed peptide in order to calculate a fold change of PhoPQ activation for each AMP.

As expected, the no-peptide control, human defensin HNP-1, and NC do not activate PhoPQ when displayed in KB1 (FIG. 25D). On the other hand, nine out of the 10 positive control peptides induce mNG expression between 8.3- and 71-fold at maximum IPTG induction (500 μM) (FIG. 25D). Notably, protegrin-1 activates PhoPQ when surface-displayed, indicating that SLAY-TCS is compatible with AMPs containing disulfide bonds. Of the positive control peptides, only Bac2a does not activate PhoPQ. In the previous study where Bac2a was reported to activate PhoPQ in S. Typhimurium, it was amidated on its C terminus, a modification that is not possible using SLAY. The data suggest that this C-terminal amidation, which also increases the net charge of the peptide by +1, may be important for Bac2a sensing by PhoPQ. To validate that activation of PhoPQ by surface-displayed AMPs is dependent on PhoQ kinase activity, this assay was repeated in KB1 PhoQ H277A. As expected, no PhoPQ activation was observed for any peptide in the PhoQ H277A strain (FIG. 25D). A slight increase in mNG fluorescence was observed at very high levels of protegrin-1 expression (FIG. 25D). However, this effect is likely due to an AMP-mediated growth slowdown resulting in mNG accumulation. Thus, SLAY-TCS enables faithful characterization of the response of PhoPQ to surface-displayed peptides, including those containing disulfide bonds.

PhoQ senses diverse human AMPs: To characterize the response of PhoQ to most human AMPs, 133 human AMPs in the Antimicrobial Peptide Database (APD3) were first identified). From this set, 16 (12%) were excluded exceeding 104 amino acids in length due to size restrictions of commercial oligonucleotide synthesis. The remaining set of 117 AMPs vary in charge, length, hydrophobicity, and secondary structure, and comprise 11 clusters of high sequence similarity. These clusters include families of mature AMPs processed from a single precursor peptide (e.g. products of cathelicidin, human defensin 5, and β-amyloid) and peptides with high sequence similarity that are produced from distinct genes (e.g. CXCL1, CXCL2, and CXCL3).

Next, a human AMP plasmid expression library was constructed wherein each plasmid encodes P_{virK}-mNG and a surface-displayed human AMP under control of P_{tac}, as in the control studies. This plasmid library was transformed into KB1. Using Illumina NGS, it was validated that sequences encoding all 117 human AMPs, along with no-peptide controls, are present in the KB1 human AMP library. Variants of human AMPs were also identified that likely resulted from errors in oligo synthesis or plasmid assembly. As expected, these variants generally had lower representation in the library than the human AMPs.

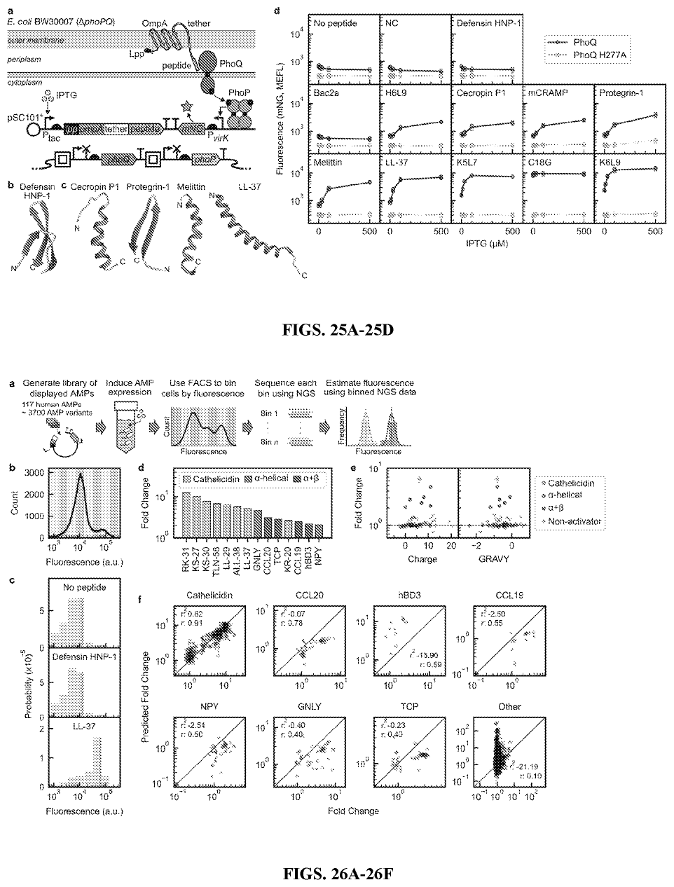

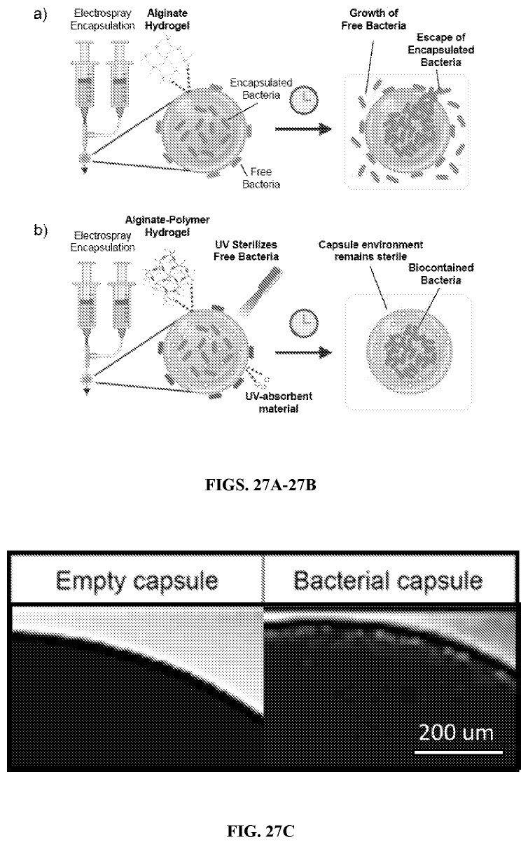

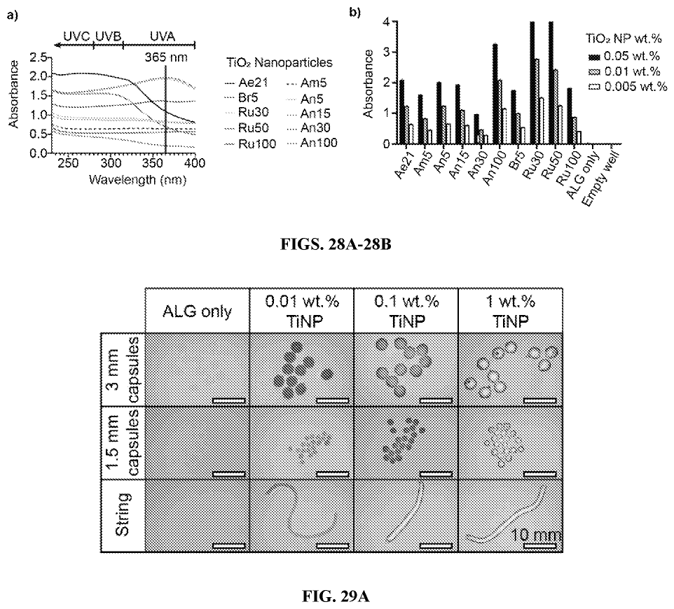

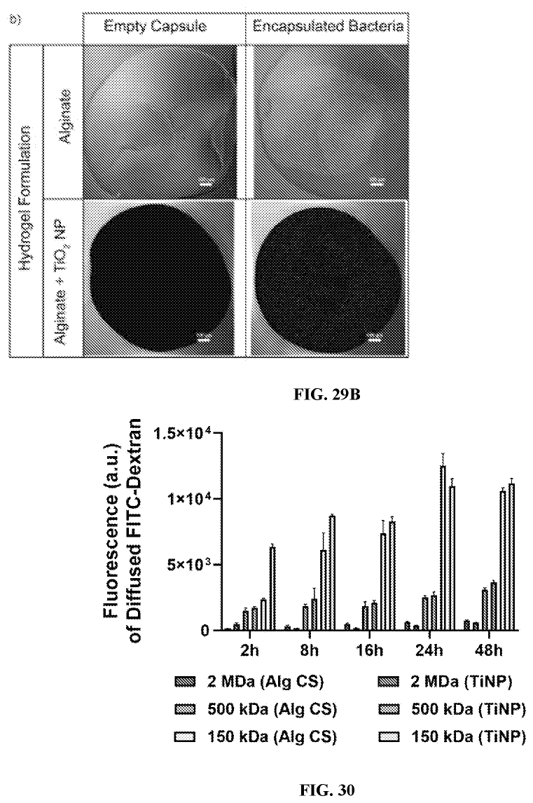

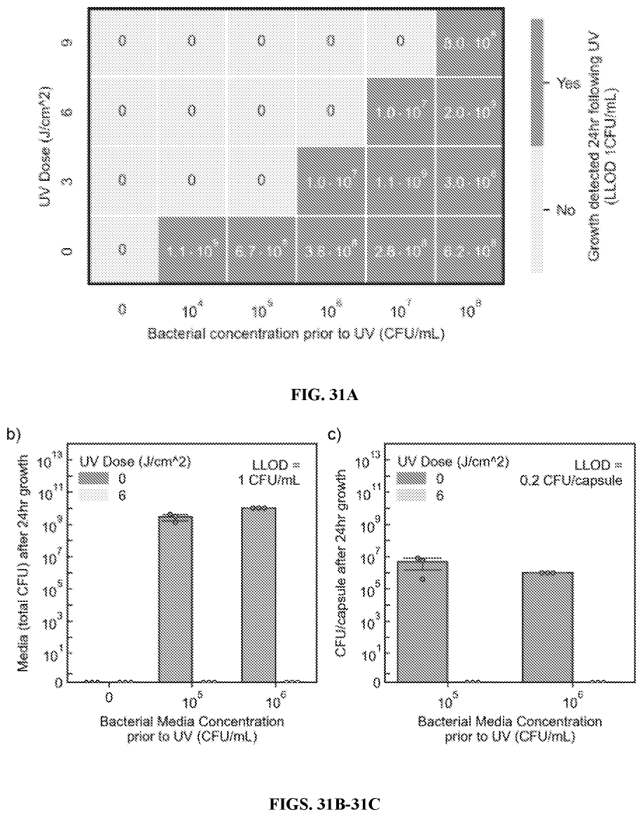

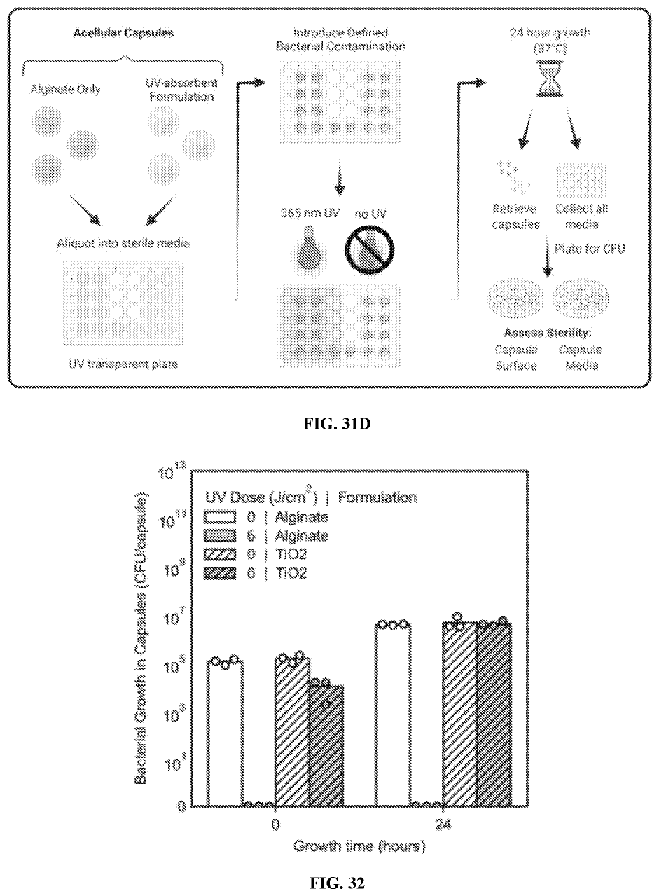

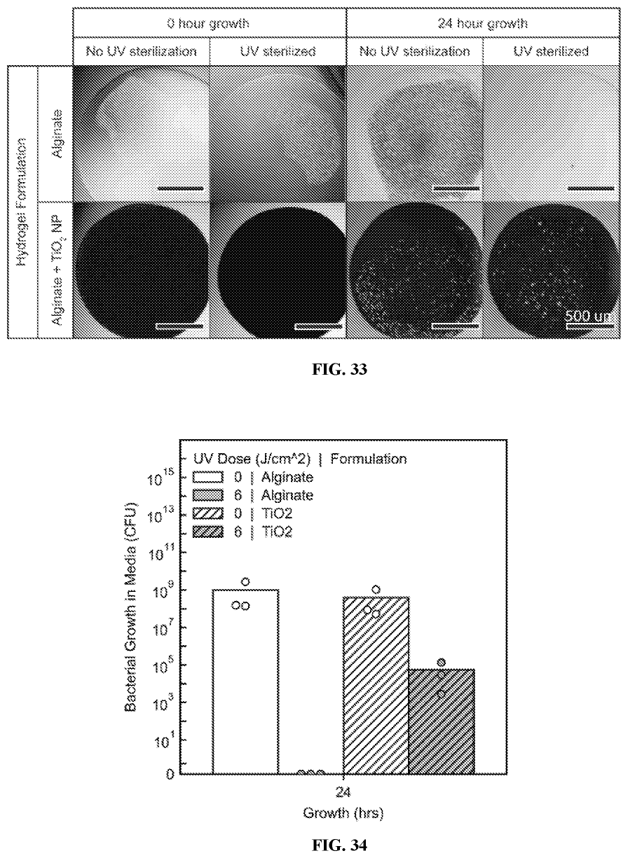

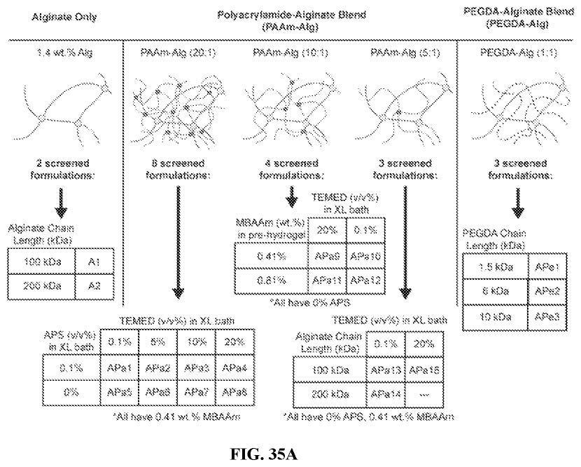

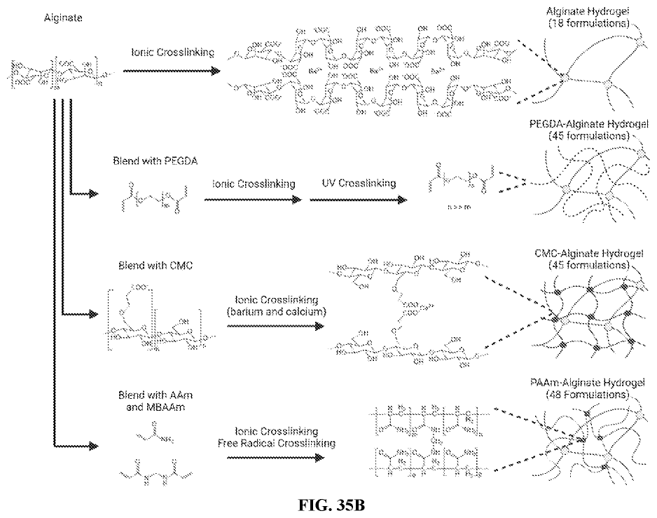

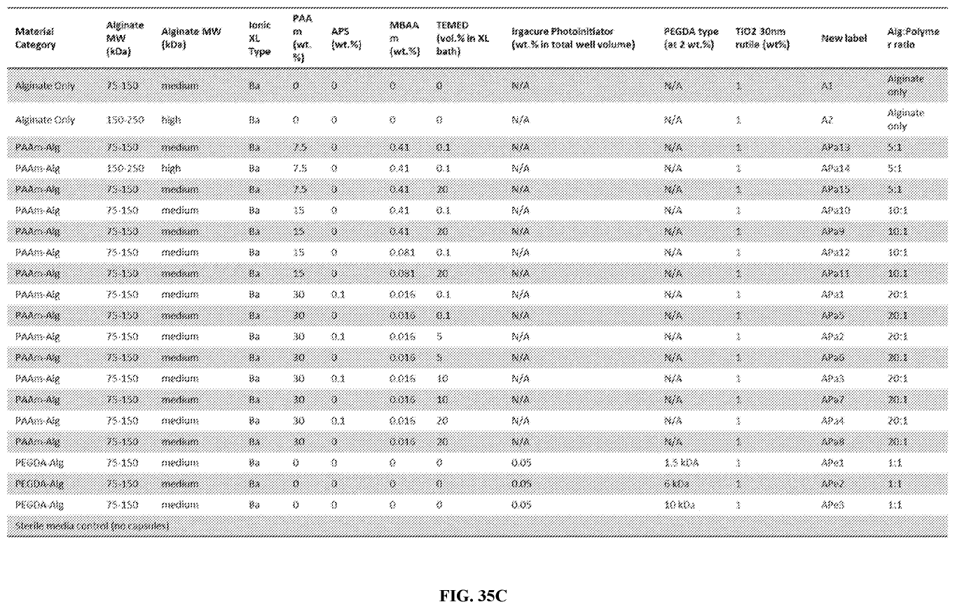

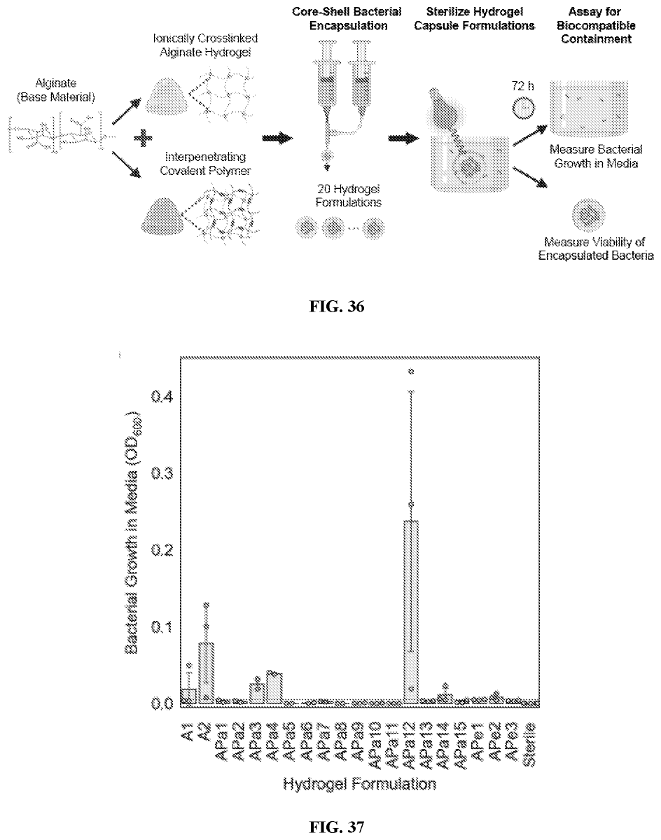

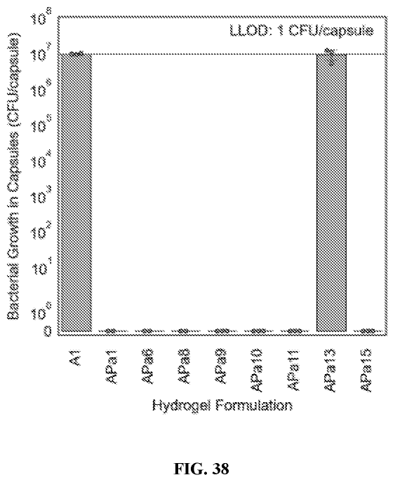

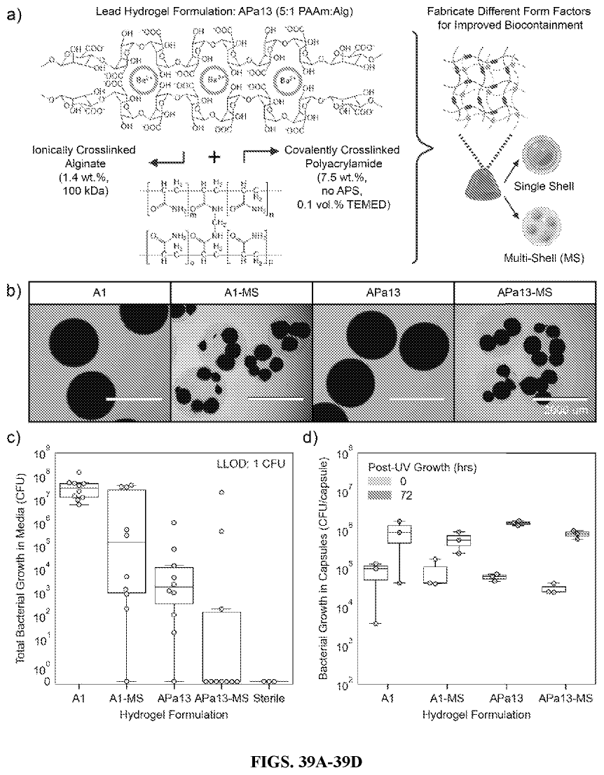

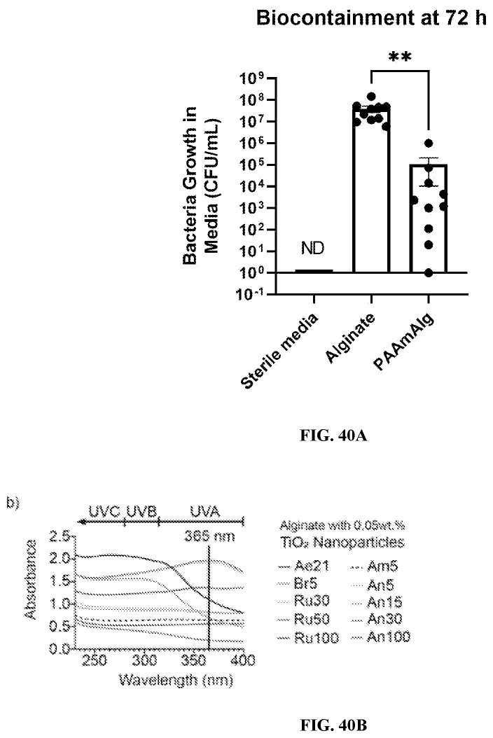

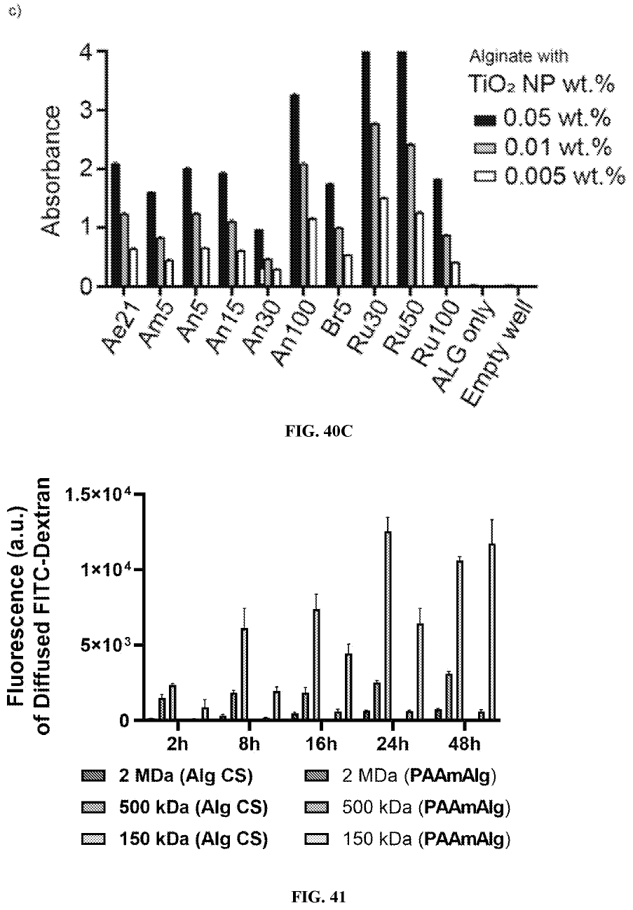

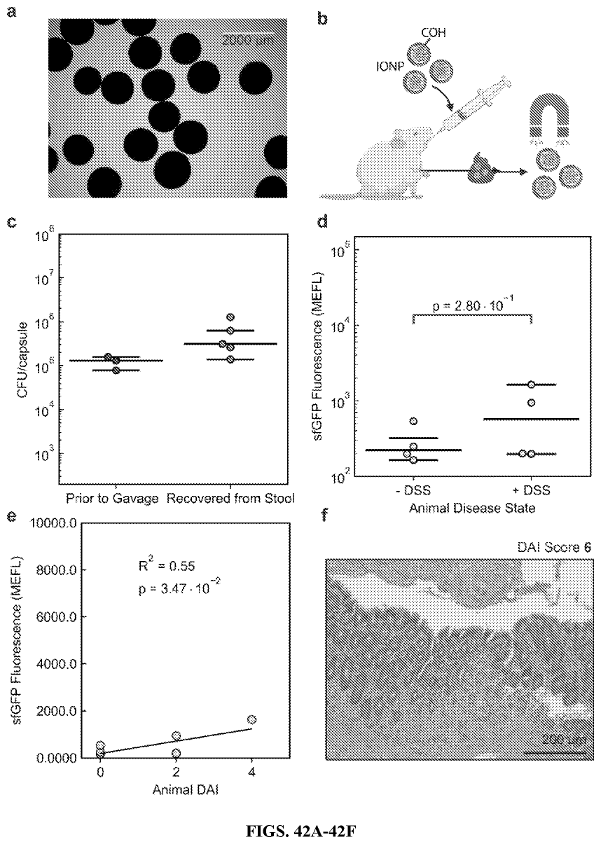

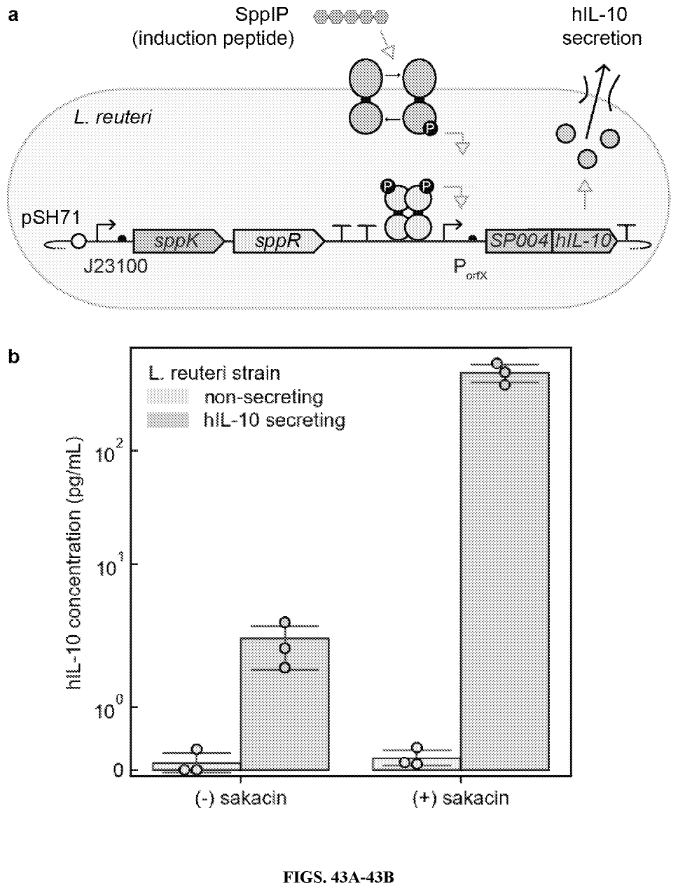

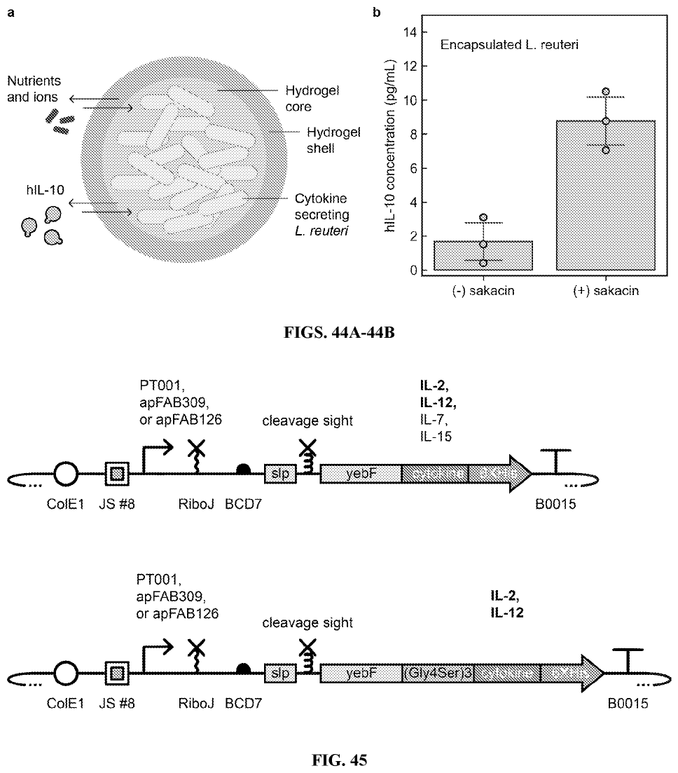

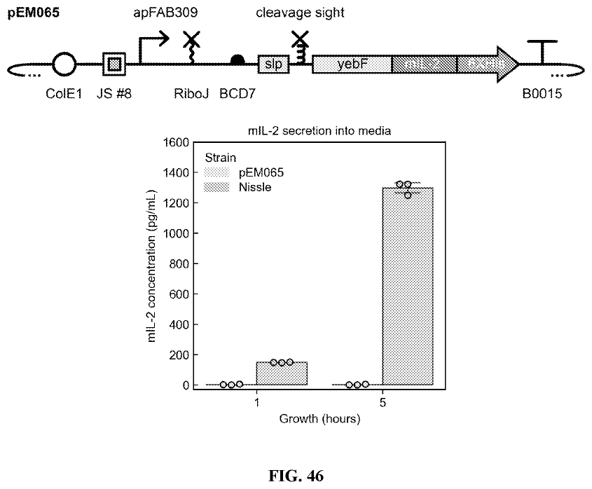

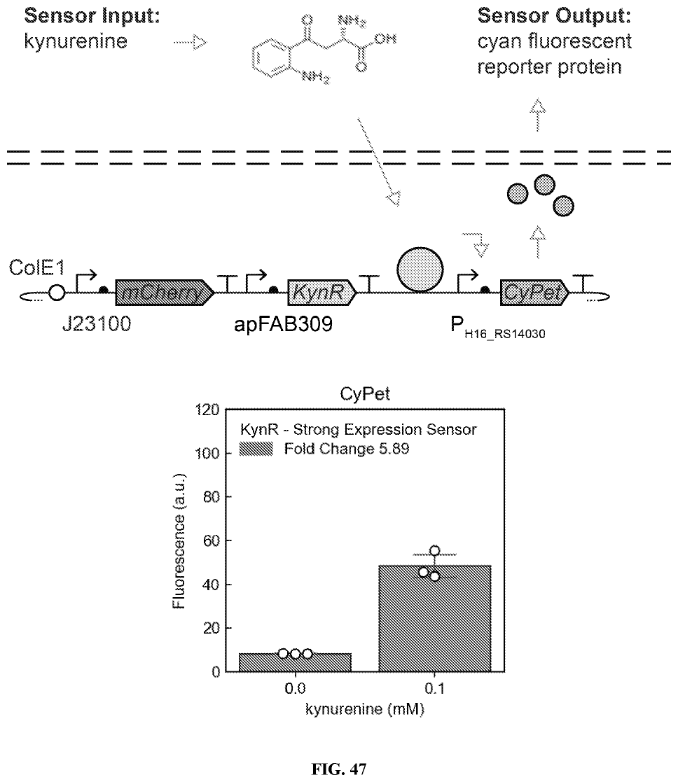

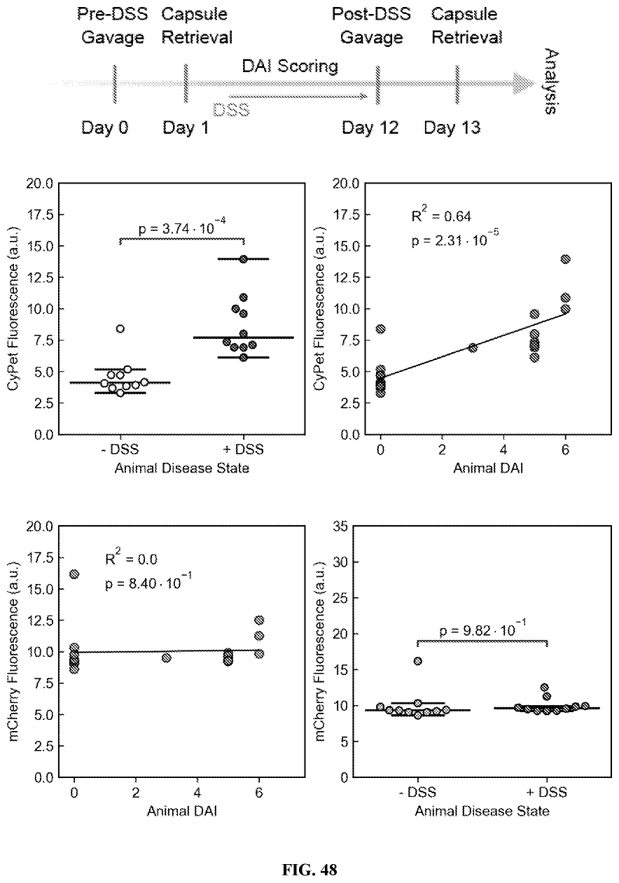

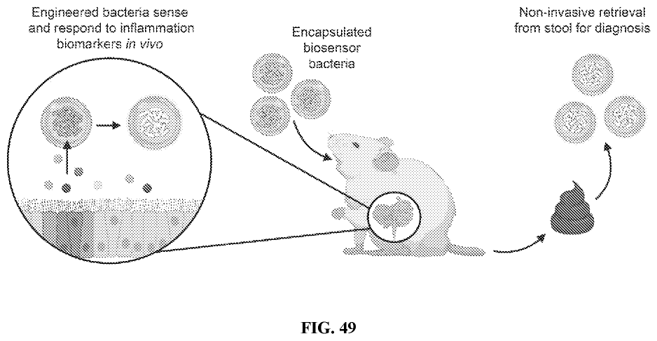

Next, sort-seq was utilized to characterize the effect of each peptide on PhoPQ activity. First, the KB1 human AMP library was grown in the presence of 500 μM IPTG, which induces sufficient expression of positive control AMPs to strongly activate PhoPQ (FIG. 25D). Then, FACS was used to sort these bacteria into eight logarithmically-spaced mNG fluorescence bins (FIG. 26B). The bins were designed to span the fluorescence distributions of negative (no peptide) and positive (C18G) PhoQ activity control strains measured on the same day. Induction of the peptide library resulted in two distinct mNG fluorescence peaks corresponding to PhoPQ activity levels of the positive and negative controls. This result suggested that some peptides in the library strongly activate PhoQ. IPTG treatment had little effect on bacterial growth, suggesting that peptide toxicity did not substantially influence the measured fluorescence values. Next, Illumina NGS was used to sequence the peptide-encoding DNA regions from bacteria in each bin and estimated the mean mNG fluorescence level of each strain using the relative abundances of peptide-encoding sequences across fluorescence bins. These estimated fluorescence values were divided by the estimated mean fluorescence of the no-peptide control to calculate the fold activation of PhoPQ in response to each peptide in the library. These analyses revealed that LL-37 activates mNG expression 5.2-fold, whereas HNP-1 does not activate mNG expression (FIG. 26C), consistent with previous work using purified peptides.

In addition to LL-37, 13 AMPs were identified that activate PhoPQ activity at least 2-fold in the sort-seq screen (FIG. 26D). Seven of these AMPs are derived from cathelicidin, the same parent protein as LL-37, and exhibit substantial sequence identity to LL-37. Only one cathelicidin-derived AMP, LL-23, failed to activate PhoPQ by 2-fold in the assay. The remaining six activators were the chemokines CCL19 and CCL20, granulysin (GNLY), human β-defensin 3 (hBD3), thrombin-derived C-terminal peptide (TCP), and neuropeptide Y (NPY), which are unrelated to cathelicidin. It was validated that each of these non-cathelicidin peptides specifically activates PhoQ when surface-displayed in KB1 in single-strain cultures. Together, these results reveal that diverse human AMPs activate PhoPQ.

### Example 4—Hydrogel-Encapsulated Bacteria to Diagnose Colon Inflammation

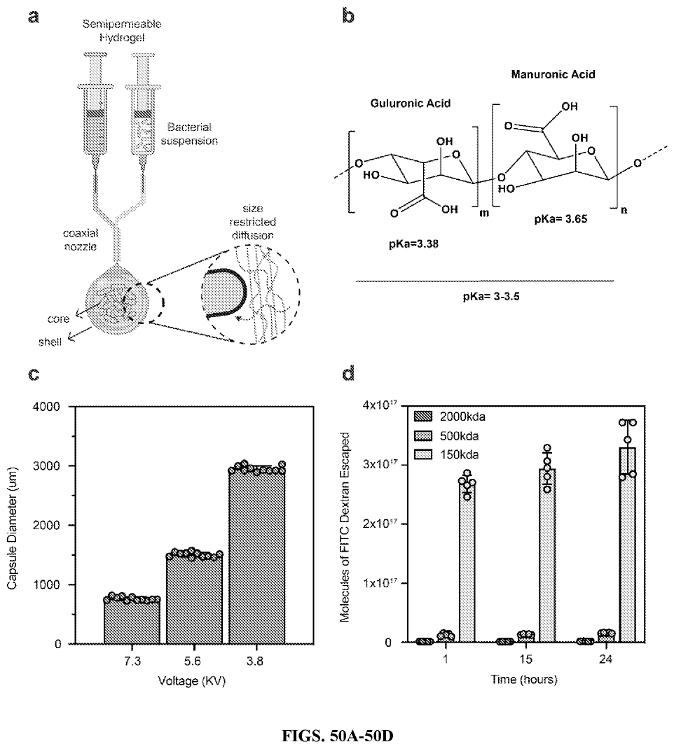

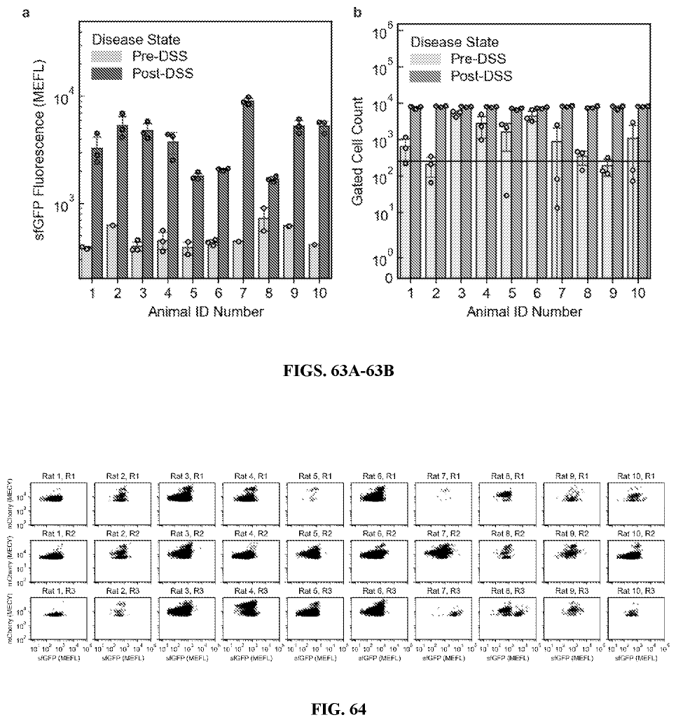

Development of a bacterial encapsulation platform that preserves cell viability. A hydrogel encapsulation method was developed for the delivery of engineered bacterial biosensors to the gastrointestinal tract (FIG. 50A). A custom-built, two-fluid co-axial electrostatic spraying device (Ghanta et al., 2020) was used to synthesize semi-permeable, core-shell bead structures wherein bacteria are loaded only into the capsule core (FIG. 50A). This system was tuneable and enabled the customization of various material properties for specific applications. An ionically crosslinked sodium alginate-based hydrogel was selected for its biocompatibility and ability to provide an environment generally conducive to good cell viability due to its pore size and ability for exchange of nutrients. Alginate hydrogels also exhibit unique pH-sensitive behavior that have established the material as an acid-resistant drug carrier (Mansourpour et al., 2015). The polymer's chemical structure included guluronic and mannuronic acid (Chuang et al., 2017), which have pKa values of 3.38 and 3.65, respectively (FIG. 50B). These pKa values cause alginate to be a negatively charged polymer at higher pH values while lower pH causes aggregation due to intramolecular hydrogen bonds that form after the carboxylic acid groups are protonated (Rizwan et al., 2017). This pH-dependent aggregation in turn enables alginate hydrogels to protect encapsulated payloads in low pH environments, including simulated gastric fluid (SGF). Various crosslinking baths can be used for the generation of alginate hydrogels. However, barium-mediated crosslinking has been shown to result in hydrogels with increased stability when in contact with physiological salt solutions and was thus utilized for the development of this platform (Morch et al., 2006). This encapsulation platform permits highly reproducible tuning of structural properties like hydrogel size, which can be reliably adjusted by modulating the voltage on the generator. For this work, a capsule diameter of 1.5 mm was desired to match the maximum clearance size from the rat GI, previously reported to be between 1-2 mm (Jang et al., 2013). A voltage of 5.6 V was found to produce 1.5 mm capsules most consistently (Standard deviation=+/−38.5 um) (FIG. 50C). Importantly, larger and smaller capsules can also be synthesized reproducibly for use in other applications. The shell thickness can also be modified by modulating the core and shell flow rate ratios. Shell thickness is an important property that enables various degrees of separation of the encapsulated cells from the host. For this work, a ratio of 5:14 was used resulting in a core radius of 0.9 mm and a shell thickness of approximately 0.6 mm (FIG. 63). The permeability of the hydrogels was assayed using dextran diffusion and the pore size was found to be approximately 500 kDa in size (FIG. 50D), which is large enough to be suitable for the size-restricted diffusion of small molecules and small enough to limit bacterial escape from the capsules. This facilitates the environmental exchange of nutrients and biomarkers between the encapsulated cells and their surrounding environment, enabling the cells to grow, multiply, and sense environmental signals in situ.

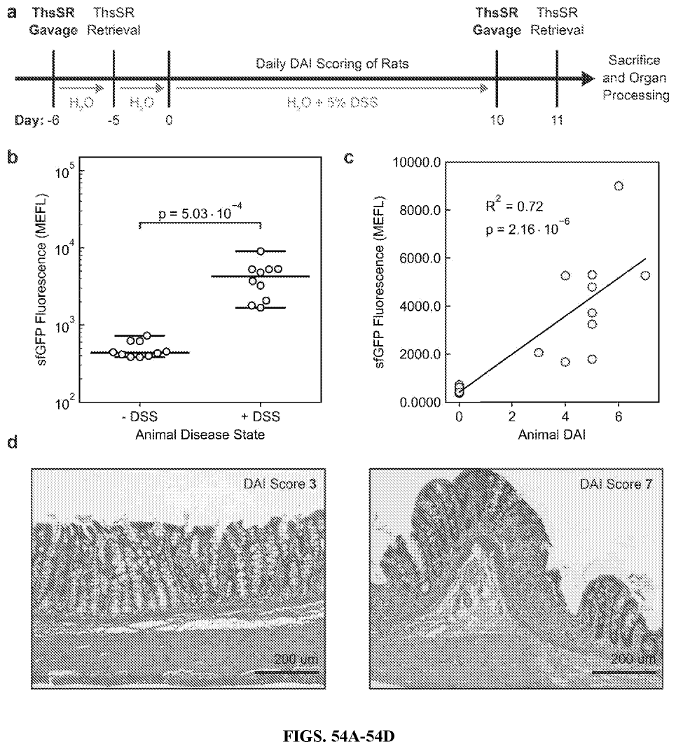

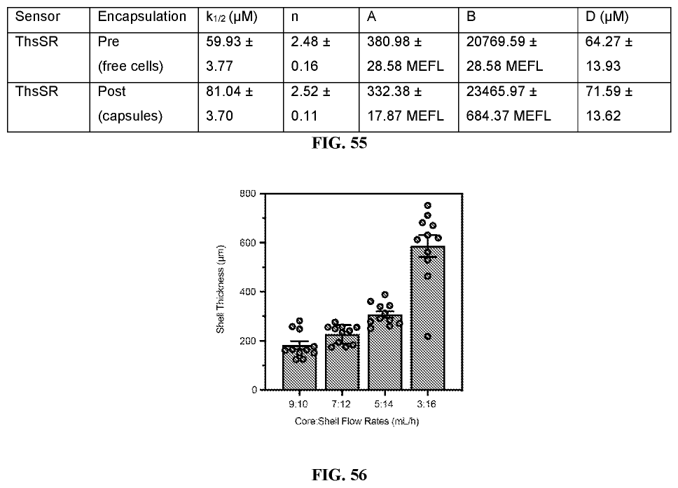

To evaluate the viability and performance of encapsulated cells, capsule-based assays were developed to leverage established microbiology methods. These included bacterial plating for live-cell assessment through counts of colony-forming units (CFU), viability staining with propidium iodide, and flow cytometry. To adapt these approaches for encapsulated cells, hydrogels were exposed to a chelating agent and physically homogenized to release bacteria from the hydrogel. EDTA is a calcium chelator that helps disrupt the alginate crosslinks and reverses gelling, allowing for less aggressive mechanical disruption to be used (Chueh et al., 2009). The duration and temperature of cell retrieval was performed such that cell viability was minimally affected and consistent among samples (FIG. 54A). The use of EDTA during homogenization improved the retrieval of encapsulated bacteria compared to PBS (FIG. 54B). The use of physical homogenization to improve hydrogel deterioration did not affect the viability of free cells (FIG. 54C).

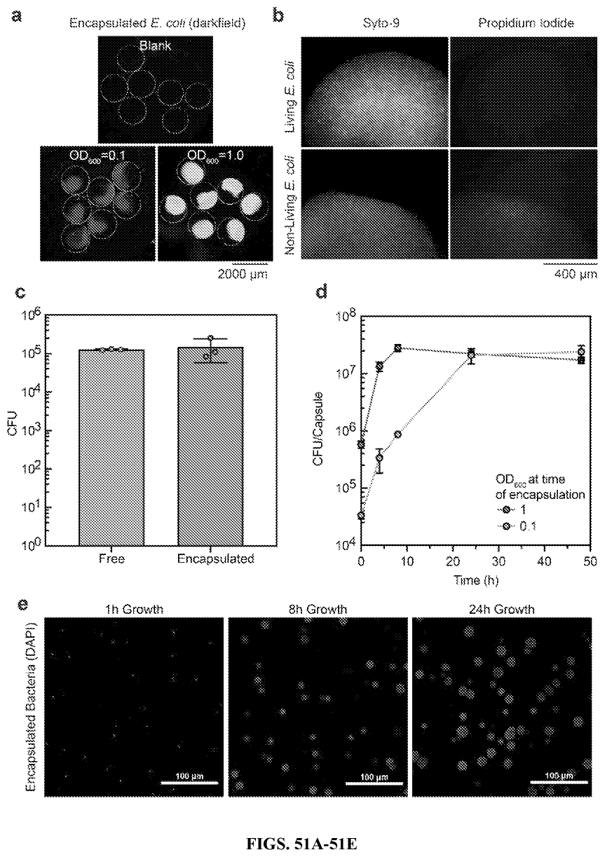

Bacterial density at the time of encapsulation was explored to determine optimal conditions for cell growth within the alginate hydrogel beads (hereafter, capsules). Encapsulated bacteria were imaged in darkfield microscopy to enhance contrast and highlight the capsule boundaries. Representative images of blank capsules and capsules loaded with OD₆₀₀=0.1 and OD₆₀₀=1 demonstrate that increases in bacterial density can be better visualized using this method. (FIG. 51A). The viability of Nissle within the capsules was assessed using Syto-9 and propidium iodide cell stains. The expected performance of the viability stains was first validated with unencapsulated, free cells (FIG. 64A). Living bacteria accept only SYTO-9 stain while non-living bacteria accept both SYTO-9 and propidium iodide. The non-living control group comprises bacteria that were heat-killed prior to encapsulation. Cell death was validated by plating for CFU (FIG. 64B). Fluorescence microscopy depicted that a majority of encapsulated bacteria remained viable, as demonstrated by the lack of red fluorescence in living cells (FIG. 51B). To quantitatively assess bacterial survival within capsules, colony forming units (CFU) were counted prior to and after encapsulation for bacteria loaded at OD₆₀₀ 0.1 (FIG. 51C). Bacterial viability (CFU) prior to and following encapsulation per volume encapsulated was not significantly different (p=0.715***). Average bacterial load per capsule is 7.47×10⁴±3.74×10⁴ CFU.

The growth of bacteria encapsulated at different phases was evaluated to investigate population dynamics within the capsule microenvironment over time (FIG. 51D). Bacteria encapsulated during exponential state at (OD₆₀₀ 0.1) exhibited a longer period of continuous growth than those encapsulated at stationary phase (OD₆₀₀ 1). The stability and robustness of engineered bacterial circuits is higher during exponential growth phase where continuous growth enables a steady state of biochemical reaction rates (Sleight et al., 2010). To deliver engineered bacteria to the animal gut, bacteria were hereafter encapsulated at OD₆₀₀ 0.1 to maintain the desired near-steady state conditions and prevent loss-of-function during transport through the gastrointestinal tract (8-10 hours for the rat model used in this work). Inside the restricted microenvironment, bacteria reached an average population capacity of 2.38×10⁷±1.19×10⁷ CFU/capsule regardless of initial growth state at the time of encapsulation. Over the course of growth, the bacteria encapsulated at OD₆₀₀ 0.1 were fixed and stained with DAPI at 0-, 8-, and 24-hours following encapsulation and imaged via confocal microscopy (FIG. 51E), illustrating that the capsule microenvironment enables cell growth.

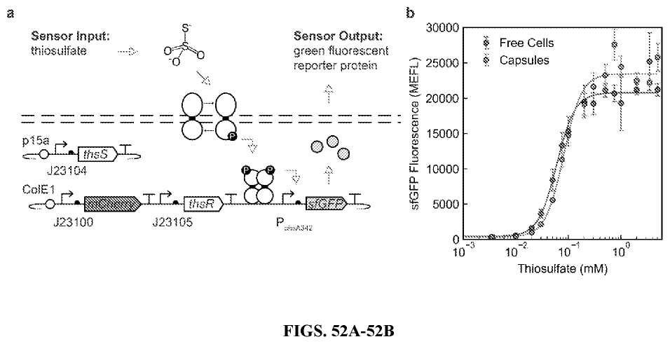

Hydrogel encapsulation preserves bacterial biosensor performance. Bacterial thiosulfate biosensors, hereafter ThsSR Nissle, were encapsulated to investigate the effects of the restricted hydrogel environment on biosensor performance. These bacteria have been genetically engineered to respond to thiosulfate by ligand-induced signaling through the Shewanella halifaxensis two-component system ThsSR as depicted in FIG. 52A (Daeffler et al. 2017). Briefly, the membrane-bound sensor kinase ThsS phosphorylates the cytoplasmic response regulator ThsR in the presence of thiosulfate. Phosphorylated ThsR then activates transcription of the fluorescent reporter sfGFP, via the promoter P_{phsA342}. This sensor strain was previously optimized for in vivo application by constitutively expressing ThsSR to eliminate the need for exogenous chemical inducers. In addition, a strong constitutive mCherry expression cassette was added to enable differentiation from native gut microbes, and the expression of ThsR was optimized to improve the biosensing performance under anaerobic conditions similar to those present in the oxygen-poor gut microenvironment.

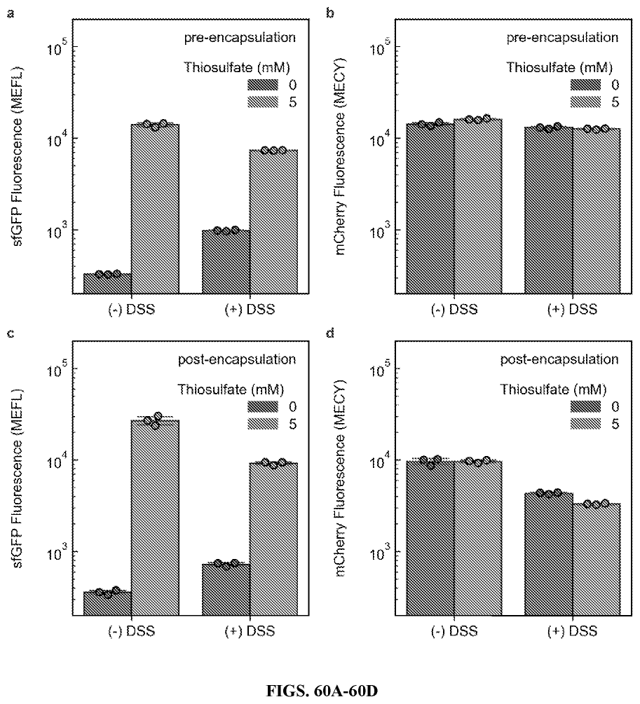

The thiosulfate dose-response of encapsulated ThsSR Nissle was compared to that of free ThsSR Nissle (FIG. 52B). The dynamic range, K_{1/2}, and hill coefficient are similar for the ThsSR sensor in both free and encapsulated cells. (FIG. 63) Biosensing capabilities were not hindered by the encapsulation process, and fluorescence of the constitutive mCherry protein remained constant across the thiosulfate dose range, further validating the expected behavior of this genetic construct (FIG. 59). Encapsulation increased the dynamic range of the sensor. The thiosulfate response of free and encapsulated ThsSR Nissle was also evaluated in the presence of 5% DSS. DSS minimally impacted the sensor response to the saturating ligand condition (FIG. 60).

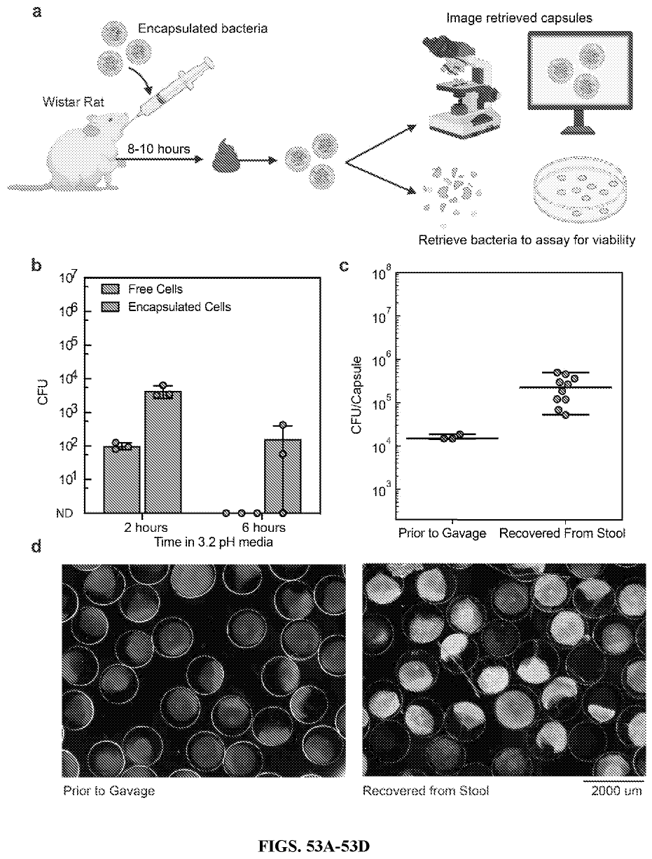

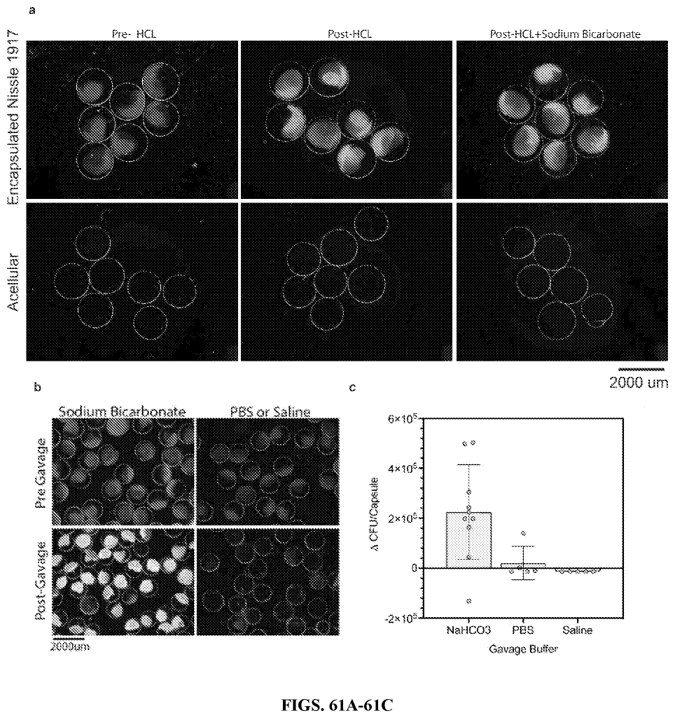

Hydrogel encapsulation preserves bacterial viability during transport through the animal gut. An important characteristic of this diagnostic platform is the ability of the hydrogel capsules and biosensor bacteria to withstand the gastrointestinal tract. Digestion involves various chemical and mechanical processes that could affect both bacterial viability and hydrogel integrity. Particularly, the acidity and bile salt concentrations of the GI have been shown to be a significant barrier to the viability of orally delivered microbes. Thus, to ensure the encapsulated biosensor bacteria could withstand digestion, Nissle were encapsulated and gavaged to healthy rats. FIG. 53A depicts the dosing regimen and post retrieval processing. The rat stomach is a very acidic environment, at an average pH of 3.2 if the rat has not been fasted (McConnell et al., 2010). The rats were not fasted for all in vivo experiments in this work. The ability of the capsules to protect bacteria in this environment was modeled by incubating encapsulated bacteria in media brought to a final pH of 3.2 with hydrochloric acid (HCl). HCl is the acidic component of gastrointestinal fluid. Capsules and free bacteria were incubated at 37 C in the acidic solution for 2 and 6 hours. Viability of encapsulated bacteria was compared to free bacteria. Results showed that encapsulated bacteria had a mean 43-fold higher viability than unencapsulated bacteria at 2 hours, and 162-fold higher at 6 hours (FIG. 53B). Representative microscopy of capsules exposed to acid depict minimal change in structure (FIG. 61A). These results suggest that viability of microbes delivered to the gut could be improved through encapsulation. For further protection, capsules were gavaged in a 10% wt/v sodium bicarbonate PBS solution. Encapsulated bacteria delivered in this solution exhibited the best survival following retrieval from the rat stool (FIG. 61C), and this gavage buffer was used for all remaining in vivo studies. Capsule morphology remains structurally similar after passage through the animal gut (FIG. 61B).

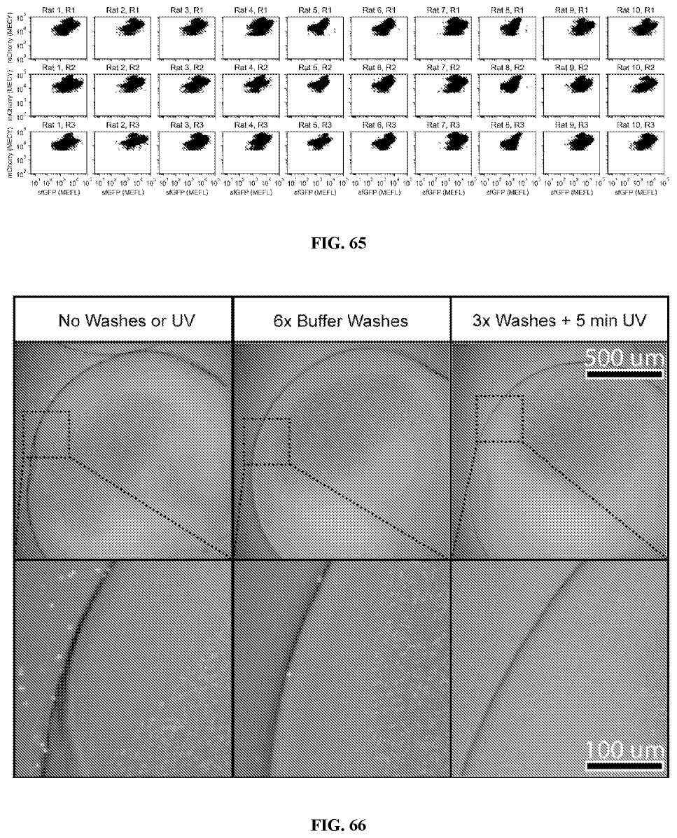

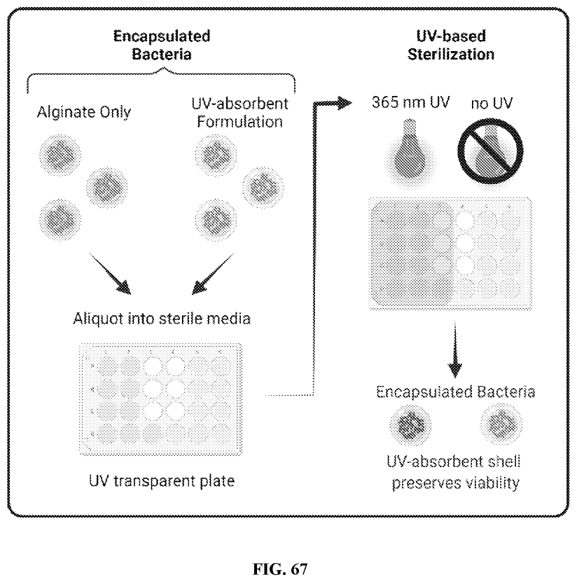

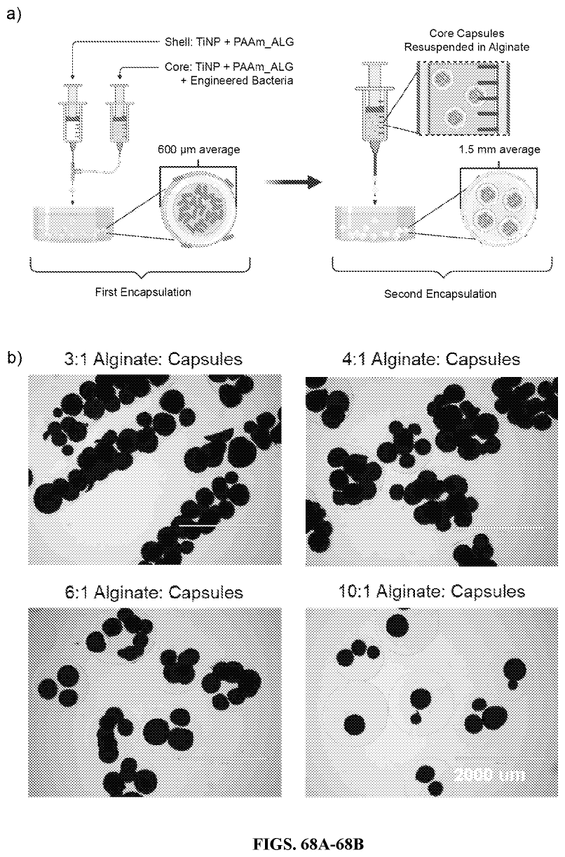

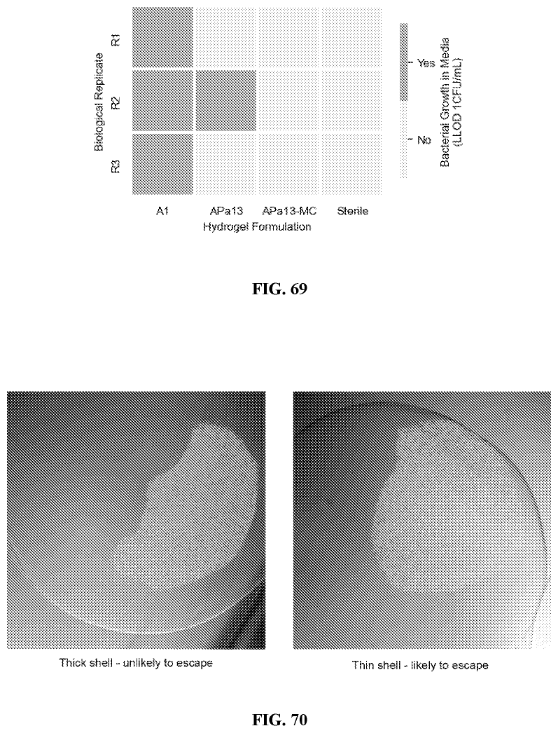

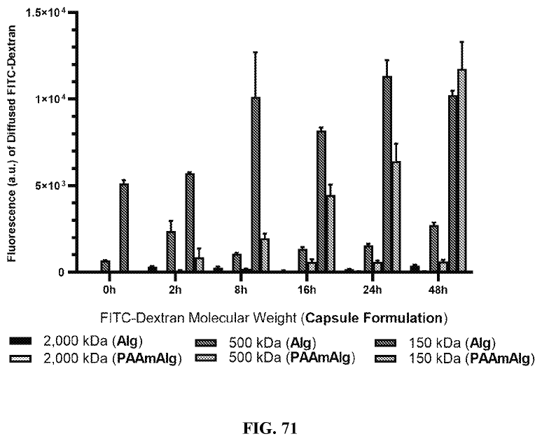

Rats were gavaged with 2 mL of hydrogel capsules in PBS, which was evaluated to have the best effect on bacterial survival compared to both PBS alone and saline (FIG. 65B). 10 hours after dosing, capsules were retrieved from fresh fecal pellets and homogenized as described above. Serial dilutions of the hydrogel-bacteria suspension were plated and CFU were counted 12 hours later. The results indicate that there was not a significant decrease in CFU per capsule prior to and following gavage (FIG. 53C). The average CFU of ThsSR was higher in the capsules recovered from stool following gavage. The increased bacterial density was also demonstrated via microscopic images where several retrieved capsules showed increased opacity when compared to capsules imaged prior to gavage (FIG. 52D). This difference in transparency is representative of increased concentrations of bacteria. These images also demonstrate that the capsules do not suffer any significant change in circularity or size. No obvious damage or breakage of the hydrogels was observed indicating. Overall, the results indicated that the encapsulated bacteria were able to pass through the gastrointestinal tract without significant effect on bacterial viability and capsule integrity, highlighting the potential of this platform as a clinically relevant diagnostic tool.

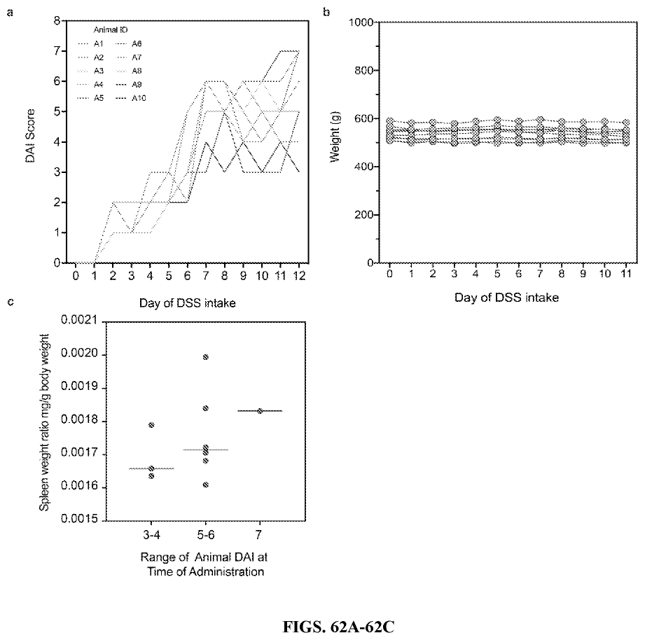

Encapsulated bacterial biosensors to diagnose chemically induced inflammation in a rat model. To assess the ability of encapsulated cells to sense and report colonic inflammation in vivo, a chemically induced disease model of gastrointestinal inflammation was established via administration of dextran sulfate sodium (DSS). DSS is a chemical irritant commonly used for the establishment of inflammation, gland loss, as well as epithelial erosion in the colon (Martin et al., 2016). It has been shown to closely resemble the state of inflammation that occurs in humans (Chassaing et al., 2014). Rats were treated with DSS in their drinking water for 12 days throughout which the disease activity index (DAI) of each rat was reported based on changes in weight, presence of occult blood in the stool, and stool consistency (FIG. 62). Encapsulated ThsSR Nissle were gavaged on day 12 post DSS treatment and retrieved 8 hours later. The timeline of treatment is depicted in FIG. 54A.

Following capsule retrieval, ThsSR Nissle were analyzed by flow cytometry as described previously (Daeffler et al., 2017). Cells were gated by an FSC/SSC scatter profile characteristic of Nissle, and remaining cells were further gated to retain only counts with an mCherry fluorescence value greater than 5000 MECY, in order to isolate only the engineered ThsSR Nissle for analysis. Bacterial samples with fewer than 250 cells following gating by these criteria were discarded. Overall, 100% of bacterial samples from DSS-treated rats were retained (FIG. 63B). Together, this data suggests that encapsulation improved the confidence of diagnosis compared to free cells by producing more tractable samples. In addition, the ease of retrievability was greatly improved by the facile collection of macroscopic capsules as compared to diffuse free cells in the stool samples. Overall, 2 orders of magnitude fewer CFU per gram body weight than previous studies (10⁷ CFU/g vs. 10⁹ CFU/g) were administered and increased efficiency was demonstrated in diagnostic capabilities through the use of the platform.

ThsSR was activated by DSS-induced gut inflammation for all 10 rats (FIG. 63A). Overall, sfGFP fluorescence significantly increased following DSS treatment (FIG. 54B). These results demonstrate that ThsSR was activated in living rats and was elevated due to DSS treatment. sfGFP fluorescence values from retrieved ThsSR Nissle correlate with animal disease state as measured by disease activity index (DAI) at the time of capsule administration (FIG. 54C), suggesting that this encapsulated biosensor system can be used to diagnose and monitor gut inflammation in this animal model.

Intestines from each rat were retrieved at sacrifice and processed for histological sectioning. Representative images of intestinal sections from rats with low (3) and high (7) DAI scores are shown, which demonstrate a relationship between DAI score and epithelial damage (FIG. 54D). Together these results demonstrate that ThsSR and KynR are effective biomarkers of gastrointestinal inflammation and correlate both to qualitative assessments of disease progression, as well as physical assessment of tissue damage.

Iron oxide nanoparticles improve retrievability of encapsulated biosensor bacteria. To improve the translation of this platform, carboxyl iron oxide nanoparticles (IONP) were incorporated into the shell compartment of the capsules (FIG. 62A). IONPs are a nanomaterial used commonly in medicine due to their established biocompatibility and stability in aqueous solutions (Arias et al., 2018). Furthermore, iron oxide nanoparticles are not charged and do not form aggregates at physiological pHs making them ideal candidates for in vivo applications (Coricovac et al., 2017). Specifically, when incorporated in the encapsulation platform, iron oxide capsules demonstrated an improved retrievability from fecal samples (FIG. 62B). To ensure that encapsulation of bacteria alongside iron oxide did not have a significant effect on bacterial viability or biosensor function in vivo, the capsules were characterized after digestion. Following a similar dosing regimen as described previously, rats treated with DSS were subsequently gavaged with 2 mL of capsules. CFU were quantified prior to and after gavage and no significant differences between viability was observed. Flow cytometry analysis of retrieved capsules showed that there was a significant increase in GFP expression from capsules retrieved from diseased rats compared to healthy rats. Furthermore, the correlation between GFP expression and disease progression as reported by DAI was similar to capsules that did not include iron oxide nanoparticles. This demonstrates that iron oxide does not inhibit biosensor function of the encapsulated bacteria. Finally, histological sections of colons demonstrated epithelial damage that relates to DAI score. Overall, the inclusion of iron oxide nanoparticles resulted in the improvement of efficiency and translation of this platform without having a detrimental effect on sensor capabilities.

Here, a delivery platform was established to improve the performance of engineered metabolite-sensing gut bacteria following oral delivery. Specifically, genetically engineered bacterial biosensors were delivered in an animal model of gut inflammation where they sensed and responded to an inflammatory biomarker. For efficient delivery to the animal gut, bacteria were delivered within a porous alginate matrix. This semi-permeable material enabled size-restricted diffusion of molecules allowing encapsulated bacteria to exchange nutrients and metabolites with their surrounding environment. This size restriction of the hydrogel further supported the containment of the engineered bacteria within their own microenvironment. Further, compared to previous methods for delivery of living diagnostics, this approach improved bacterial survival and function in the gastrointestinal tract, enabled improved in vivo biocontainment, and facilitated rapid post-digestion retrievability and analysis. The encapsulated bacteria were administered orally and retrieved from stool for diagnosis of gastrointestinal inflammation. Retrieval of bacteria was further improved by the employment of carboxyl iron oxide nanoparticles which enabled magnetic retrieval of the capsules from fecal samples without significant effects on biosensor function. Overall, this platform allowed us to measure disease pathology in an objective, and non-invasive manner as opposed to conventional IBD diagnostic measures which are subjective or in severe cases rely on invasive and painful colonoscopy procedures and tissue biopsy. Furthermore, the non-invasive route of administration of the device results in the ability for frequent dosing and monitoring of flareups that will improve the rate of diagnosis and disease management for patients.

Overall, this technology may provide significant advances for the clinical standards of care, as well as the administration and future approval of living bacterial diagnostics as tools to sense and respond to the gut microenvironment. The innovations presented herein will improve the generalizability and applicability of synthetic biology approaches for human health applications. Specifically, the delivery and viability of probiotics to patients is significantly improved, highlighting a readily available avenue for clinical translation. Living microbial diagnostics that can detect inflammation in situ could overcome many challenges associated with the current standard of care for IBD. In addition to IBD, the mammalian colon is an important target for many other localized and systemic conditions. The human metabolism, immune, and brain function are all affected by gut processes, which are orchestrated in part by metabolic and signaling interactions among host cells and the resident microbiota. Dysbiosis in the gut microbiome is tied to many aspects of human health, and genetically engineered sensor bacteria have untapped potential as tools to analyze, target, and treat this dynamic and inaccessible microenvironment. The ability to accomplish these goals in a clinically translational manner is significantly improved by the technology described herein.

TABLE 1
Details and abbreviations for screened titanium dioxide nanoparticles (TiNP).
TiNP is a highly UV absorbent material that can be suspended
in alginate solutions and hydrogels.

| Type and Size | Abbreviation | Product | Supplier |
|---|---|---|---|
| 21 nm Anatase and | Ae21 | 718467 | Sigma-Aldrich |
| Rutile | | | |
| 5 nm Amorphous | Am5 | US3501 | U.S. Research Nanomaterials, Inc. |
| 5 nm Anatase | An5 | US3838 | U.S. Research Nanomaterials, Inc. |
| 15 nm Anatase | An15 | US3492 | U.S. Research Nanomaterials, Inc. |
| 30 nm Anatase | An30 | US3498 | U.S. Research Nanomaterials, Inc. |
| 100 nm Anatase | An100 | US3411 | U.S. Research Nanomaterials, Inc. |
| 5 nm Brookite | Br5 | 791326 | Sigma-Aldrich |
| 30 nm Rutile | Ru30 | US3520 | U.S. Research Nanomaterials, Inc. |
| 50 nm Rutile | Ru50 | US3530 | U.S. Research Nanomaterials, Inc. |
| 100 nm Rutile | Ru100 | US3535 | U.S. Research Nanomaterials, Inc. |

### Example 4—Materials and Methods

DNA and bacterial strains. E. coli Nissle 1917 was used for viability staining experiments. Strain KD01 was used for all other experiments. KD01 is an engineered thiosulfate sensing E. coli Nissle 1917 strain that is resistant to chloramphenicol and spectinomycin, as described in Daeffler et al. Freezer stocks of plasmid strains were prepared by growing a colony containing sequence-verified plasmids in LB Miller broth and the appropriate antibiotics (35 μg/ml chloramphenicol and 100 μg/ml spectinomycin) to OD₆₀₀=˜0.5, adding glycerol to a 15% v/v final concentration, and freezing at −80° C. All experiments were performed aerobically.

Bacterial preparation for encapsulation. Overnight cultures were started from freezer stocks in LB with the appropriate antibiotics and incubated shaking for 18 h at 250 rpm at 37° C. Cultures were then diluted 100× into fresh LB with appropriate antibiotics and incubated shaking at 250 rpm at 37° C. to OD₆₀₀=0.1. Cultures were placed on ice to stop cell growth, and remaining procedure was performed on ice or at 4° C. Cultures were transferred into sterile, ice-cold tubes and centrifuged at 4000 g for 10 min. Cells were washed once with sterile, ice-cold PBS, to remove traces of LB. Cells were then concentrated 10× into a 1:19 solution of PBS:alginate (1.4% SLG20 alginate, ProNOVA, diluted in 0.8% saline). The resulting bacterial alginate suspension was used as the core solution for the encapsulation procedure. All capsules prepared in this work were formed with a core-shell structure where bacteria are loaded only into the core, with an average core radius of 450 um, such that the capsule core volume is ˜ 0.38 μL. For free cell experiments, the cells were concentrated into a 1:19 solution of PBS: LB rather than PBS:alginate, and 1.9 μL of the resulting bacterial suspension was used to model the bacterial load of 5 capsules.

Bacterial encapsulation within alginate hydrogels. The bacterial alginate suspension prepared as described was loaded into a 5 mL Luerlock syringe to be used for the capsule core. Another identical syringe was loaded with SLG20 alginate to be used for the capsule shell. Alginate hydrogel capsules were synthesized using a custom-built, two-fluid co-axial electrostatic spraying device. The device consists of a voltage generator attached to the tip of a co-axial needle and grounded to a 1:4 BaCl₂: mannitol crosslinking bath. The co-axial needle is fed by the 2 syringes described, which are placed in 2 separate syringe pumps and suspended over 150 mL of the crosslinking bath. For this work, the ratio between the flow rates of the core and shell syringe was set to 5:14 for a shell thickness of ˜0.30 mm. Capsules drop into the crosslinking bath as they are synthesized, and incubate for 15 min. The crosslinking bath is then decanted, capsules are washed 3× with HEPES buffer, and transferred to Ca+KREBS (128 mM NaCl, 5 mM KCl, 2.7 mM CaCl₂, 1.2 mM MgCl₂, 1 mM Na₂HPO₄, 1.2 mM KH₂PO₄, 5 mM NaHCO₃, 10 mM HEPES-henceforth, capsule buffer).

Dextran diffusion assays for permeability. 150 kda (Sigma), 500 kda (Sigma), and 2000 kda (Sigma) dextran's were encapsulated into the core of 1.4% SLG20 core-shell capsules using the encapsulation protocol. Specifically, 5 ul of 25 mg/mL dextran per mL of alginate. From each group, 5 capsules per well of a 96 well plate were placed and collected the supernatant at 1, 15, and 24 hours and transferred it into an adjacent well (with no capsule) and read the fluorescence on a TECAN Plate reader (with excitation and emission 490/521). The fluorescence was normalized to molecules of dextran per well at each time point.

Bacterial viability staining. Staining of free cells was performed according to the manufacturer's guidelines using the LIVE/DEAD® BacLight™ Bacterial viability kit (Thermo Fisher). Additional free cell samples were prepared to test each stain independently as an assay control (FIG. 57A). Staining of the encapsulated cells was performed as follows: 10 capsules per replicate were washed 3× with sterile 0.9% saline, placed in 1 mL saline, and incubated on ice for 40 min. The nonliving control groups were incubated in a water bath at 65-70° C. for 40 min prior to staining. 5 capsules from each replicate were removed and processed as described below to determine the living bacterial concentration (FIG. 2B). The remaining 5 capsules were placed into 200 μL saline. The remaining procedure was performed according to vendor instructions, with the following modifications: the live/dead staining solutions were dissolved in saline, and the capsules were washed 3× with 1 mL saline prior to imaging due to high stain absorption by the alginate.

Capsule microscopy. Prior to imaging, capsules were washed 3× and transferred into ˜5 mL capsule buffer within a 6-well plate. Dark- and bright-field microscopy was performed using an EVOS at 2× magnification. Fluorescent microscopy images were obtained using an EVOS XL at 10× magnification. For SYTO-9 fluorescence, an excitation/emission wavelength of 480 nm/500 nm was used, and 490 nm/635 nm for propidium iodide and mCherry fluorescence. Images in this work are representative of 3 replicates for in vitro bacterial experiments and 10 replicates for in vivo rat experiments.

Hydrogel capsule processing for bacterial assays. For all bacterial assays, capsules were first processed to release encapsulated bacteria and enable measurements. Each experimental condition was performed in triplicate. This procedure was performed on ice or at 4° C. For each replicate, 5 capsules were transferred in capsule buffer to sterile flow cytometry tubes. For the remaining processing procedure, only 3 samples were processed at a time, to avoid cell over-exposure to chelating agents. Capsule buffer was aspirated from the capsules and replaced with 500 μL of EDTA (pH 8). Capsules were then homogenized using an electric homogenizer blade at speed 3 for 10-20 seconds until no visible capsule fragments remain (henceforth, the homogenized capsule sample). The homogenizer blade is cleaned and sterilized prior to the first sample, in between samples, and after the last sample. Assays using the homogenized sample were performed immediately following this processing procedure.

Bacterial biosensing assays. To determine the response of the biosensing bacteria to thiosulfate, bacteria were first encapsulated as described. 5 capsules in capsule buffer were then aliquoted into wells in a sterile 96-well plate. Capsule buffer was aspirated from each well and replaced with 200 μL of M9+glycerol (1× M9 salts, 0.4% v/v glycerol, 0.2% casamino acids, 2 mM MgSO₄, and 100 μM CaCl₂) at varying concentrations of sodium thiosulfate pentahydrate (Sigma Aldrich). For free cell experiments, bacterial suspensions were prepared as described to model the bacterial alginate suspension, and 1.9 μL of bacterial suspension was used to inoculate each well. The 96-well plate was incubated stationary at 37° C. for 6 h and then placed in an ice water bath to stop cell growth. Remaining procedure was performed on ice or at 4° C. Capsules were then processed as described. The homogenized samples were then passed through Bel-Art SP Scienceware Flowmi 40 μm cell strainers (Fisher Scientific) to remove solids. The filtered samples were then diluted 10× into 1 mL ice-cold PBS for flow cytometry analysis. For free cell experiments, 1.9 μL of the final experimental culture was added to 500 μL EDTA and diluted into PBS as for the capsule samples. Reported fluorescence values are not corrected for cellular autofluorescence.

Flow cytometry and data analysis. Flow cytometry analysis was performed on a BD FACScan flow cytometer with a blue (488 nm, 30 mW) and yellow (561 nm, 50 mW) laser. Fluorescence was measured on two channels: FL1 with a 510/20-nm emission filter (GFP), and FL3 with a 650-nm long-pass filter (mCherry). For in vitro bacterial experiments, cells were thresholded by an SSC scatter profile characteristic of E. coli Nissle 1917. Cells were further thresholded in the FL3 channel, to ignore counts with low mCherry-like fluorescence that arise from alginate micro-fragments remaining after capsule homogenization. Typical event rates were between 1,000 and 2,000 events per second for a total of 10,000 events within the gated population. For in vivo experiments, data were collected for 5 min or for 10,000 counts within the gated population, whichever came first. Calibration particles (Spherotech, catalog RCP-30-20A) were run at the end of every experiment at the same gain settings used for data collection. Following data acquisition, raw data were processed using FlowCal (Castillo-Hair et al., 2016). First, a standard curve was generated from the calibration beads to convert arbitrary units from the cytometer into absolute fluorescence units (MEFL for FL1 and MECY for FL3). Then, data were gated by an FSC/SSC scatter profile characteristic of E. coli Nissle 1917. Remaining cell counts were further gated to retain only counts with an FL3 value greater than 5000 MECY. Samples giving fewer than 250 counts by these gating procedures would be discarded. Overall, capsules retrieved from DSS-treated rats gave more counts/sample than those retrieved from rats prior to DSS treatment.

Bacterial viability assays. To determine the viable bacterial capsule load following an experiment, the homogenized capsule sample is vortexed at speed 6 and immediately diluted into PBS. Serial dilutions of the homogenized capsule sample were prepared in PBS and plated onto LB agar with appropriate antibiotics and incubated at 37° C. for ˜18 h. For free cells, 1.9 L of the final experimental culture was added to 500 μL EDTA and diluted in PBS as for the capsule samples. Bacterial colonies were counted to determine the concentration (CFU/mL) of the homogenized capsule sample and to calculate the bacterial capsule load (CFU/capsule).

Dextran sodium sulfate rat experiments. All animal studies were performed in accordance with guidelines established by the Animal Welfare Committee. Sprague Dawley rats were weighed on day 0, prior to any manipulation. Stool was collected and checked for occult blood. Capsules were retrieved from stool samples of healthy rats at this stage. To establish intestinal inflammation, rats were provided drinking water with 3-5% wt/v dextran sulfate sodium for 12 days. The body weight, stool consistency and presence of gross bleeding or occult blood in feces for each animal were scored daily from 0-4. The score in each category was summed to determine the animals overall disease activity index (DAI); no weight loss, normal stool consistency, negative hemoccult: 0; 1-5% weight loss, positive hemoccult but no visible blood, loose/soft stools: 1; 5-10% weight loss, positive hemoccult with visual pellet bleeding, very soft stools: 2; 10-15% weight loss, blood around the rectum/prolapse, watery stools: 3. 15-20% weight loss, gross bleeding, and diarrhea:4. After 12 days rats were dosed with 2 mL of encapsulated bacteria in 5 ml of PBS containing 10% wt/v sodium bicarbonate via oral gavage. Ten to twelve hours following gavage, rats were separated, and fecal samples were collected, capsules were retrieved, and transferred to PBS on ice.

Stool and intestinal sample preparation. Stool samples were collected from each rat prior to DSS administration and again after 12 days of DSS administration. 10 capsules were retrieved from each of 3 separate stool samples from each rat. For each of the 3 stool replicates, 5 capsules were homogenized and processed as described for the bacterial biosensing assays. The remaining 5 capsules were imaged to qualitatively assess capsule integrity following passage through the rat GI tract. From the 3 homogenized capsule samples for each rat, 1 was plated as described for the bacterial viability assays. The 3 homogenized capsule samples were processed for flow cytometry as described previously in the biosensing assays. At the time of sacrifice, the colon was separated from the cecum and rectum at the distal and proximal ends respectively. The contents of the colon were subsequently rinsed out with 10 mL of cold PBS via polyurethane gavage tubing inserted into the distal end of the colon. The colon was then cut longitudinally along the mesenteric line and rinsed in a petri dish containing cold PBS as described previously (Bialkowska et al., 2016). With the luminal side facing upwards, the colon was swiss rolled against a wooden stick beginning with the proximal end. The rolled colon was placed in a tissue cassette and stored in 10% formalin for 24 hours. Then, the cassettes were transferred to 70% ethanol for 24 hours and submitted for sectioning and H&E staining.

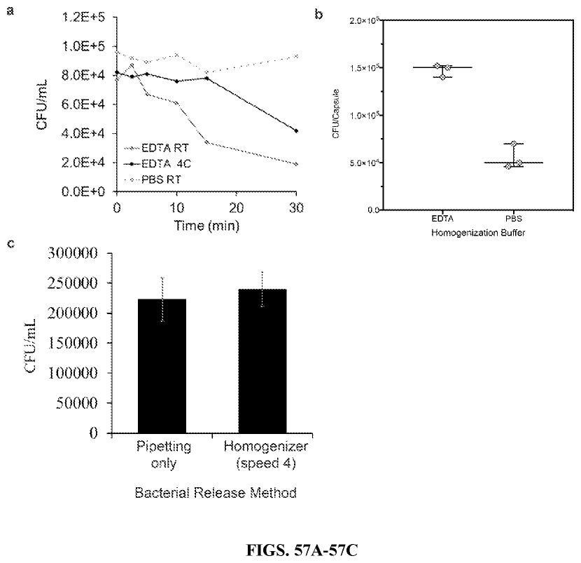

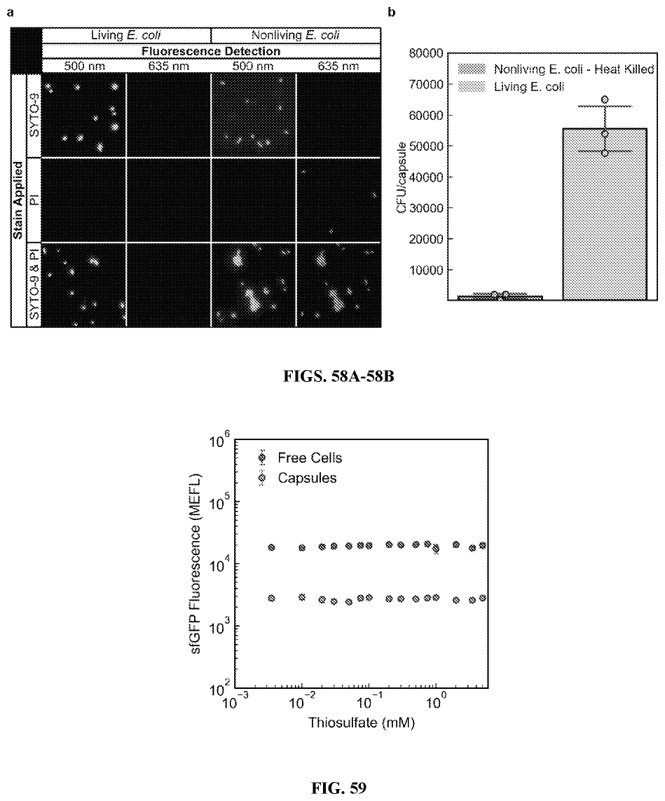

Transfer function modeling and parameter estimation. The transfer functions were obtained by fitting the averaged fluorescence values at each ligand concentration to the Hill equation, F=A+B/(1+(k_{1/2}/L)ⁿ), using the LmFit python package for non-linear least-squares minimization and curve-fitting (Newville et al., 2016). Here, F is the fluorescence at a given ligand concentration L, k_{1/2} is the concentration of ligand that elicits a half-maximal response, n is the Hill coefficient, A is the fit of the minimum response with no ligand, and B is the fit of the maximum response at saturating ligand concentration. All transfer functions were measured for 3 independent biological replicates. Datasets from each replicate were fit independently. The 95% confidence intervals of fit parameter values were calculated using the conf_interval function in LmFit, which executes the F-test. Fit parameters for all experiments in this study are shown in FIG. 56. The dynamic range was measured as the ratio between the maximal and minimal response.

Statistics and data analysis. Statistical analysis was performed using the SciPy python package (Virtanen et al., 2020) and data was visualized using the Seaborne python package (Waskom, 2021). Equal sample variance was assumed for all datasets where variances were within a twofold magnitude of one another. The Shapiro-Wilk test for normality was administered to the data for each pre- and post-DSS comparison. Any comparison for a non-normal distribution was performed with the non-parametric Mann-Whitney U-test. For in vivo studies, correlation was evaluated using the Pearson correlation coefficient to measure the linear relationship between bacterial fluorescence and rat DAI. The corresponding p-values were calculated assuming a normal distribution. Data in FIGS. 50, 51, 56, 59, 60, and 62 was visualized using GraphPad Prism. Flow cytometry scatter plots were visualized using FlowCal (Castillo-Hair et al., 2016). Schematics were created in part with BioRender.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arias, L. S. et al. Iron Oxide Nanoparticles for Biomedical Applications: A Perspective on Synthesis, Drugs, Antimicrobial Activity, and Toxicity. Antibiot. (Basel, Switzerland) 7, (2018).
Bialkowska, A. B., Ghaleb, A. M., Nandan, M. O. & Yang, V. W. Improved Swiss-rolling Technique for Intestinal Tissue Preparation for Immunohistochemical and Immunofluorescent Analyses. J. Vis. Exp. 2016, (2016).
Bochenek, M. A. et al. Alginate encapsulation as long-term immune protection of allogeneic pancreatic islet cells transplanted into the omental bursa of macaques. Nat. Biomed. Eng. 2, 810-821 (2018).
Castillo-Hair, S. M. et al. FlowCal: A User-Friendly, Open Source Software Tool for Automatically Converting Flow Cytometry Data from Arbitrary to Calibrated Units. ACS Synth. Biol. 5, 774-780 (2016).
Chassaing, B., Aitken, J. D., Malleshappa, M. & Vijay-Kumar, M. Dextran sulfate sodium (DSS)-induced colitis in mice. Curr. Protoc. Immunol. 104, (2014).
Chuang, J. J. et al. Effects of pH on the Shape of Alginate Particles and Its Release Behavior. Int. J. Polym. Sci. 2017, (2017).
Chueh, B. H. et al. Patterning alginate hydrogels using light-directed release of caged calcium in a microfluidic device. Biomed. Microdevices 2009 121 12, 145-151 (2009).
Coricovac, D. E. et al. Biocompatible colloidal suspensions based on magnetic iron oxide nanoparticles: Synthesis, characterization and toxicological profile. Front. Pharmacol. 8, 154 (2017).
Daeffler, K. N. et al. Engineering bacterial thiosulfate and tetrathionate sensors for detecting gut inflammation. Mol. Syst. Biol. 13, 923 (2017).
Dai, Z. et al. Versatile biomanufacturing through stimulus-responsive cell-material feedback. Nat. Chem. Biol. 15, 1017-1024 (2019).
Ghanta, R. K. et al. Immune-modulatory alginate protects mesenchymal stem cells for sustained delivery of reparative factors to ischemic myocardium. Biomater. Sci. 8, 5061-5070 (2020).
Hsu, B. B. et al. In situ reprogramming of gut bacteria by oral delivery. Nat. Commun. 11, 5030 (2020).
Jang, S. F. et al. Size discrimination in rat and mouse gastric emptying. Biopharm. Drug Dispos. 34, 107-124 (2013).
Mansourpour et al. Development of Acid-Resistant Alginate/Trimethyl Chitosan Nanoparticles Containing Cationic β-Cyclodextrin Polymers for Insulin Oral Delivery. AAPS Pharm Sci Tech 2015 164 16, 952-962 (2015).
Martin, J. C., Bériou, G. & Josien, R. Dextran Sulfate Sodium (DSS)-Induced Acute Colitis in the Rat. Methods Mol. Biol. 1371, 197-203 (2016).
McConnell, E. L., Basit, A. W. & Murdan, S. Measurements of rat and mouse gastrointestinal pH, fluid and lymphoid tissue, and implications for in-vivo experiments. J. Pharm. Pharmacol. 60, 63-70 (2010).
Mørch, Ý. A., Donati, I., Strand, B. L. & Skjåk-Bræk, G. Effect of Ca2+, Ba2+, and Sr2+ on alginate microbeads. Biomacromolecules 7, 1471-1480 (2006).
Morkel, M., Riemer, P., Bläker, H. & Sers, C. Similar but different: distinct roles for KRAS and BRAF oncogenes in colorectal cancer development and therapy resistance. Oncotarget 6, 20785-20800 (2015).
Neurath, M. F. Targeting immune cell circuits and trafficking in inflammatory bowel disease. Nat. Immunol. 20, 970-979 (2019).
Newville, M. et al. Lmfit: Non-Linear Least-Square Minimization and Curve-Fitting for Python. ascl ascl:1606.014 (2016).
Riglar, D. T. et al. Live Diagnostics of Inflammation. 35, 653-658 (2018).
Rizwan, M. et al. pH Sensitive Hydrogels in Drug Delivery: Brief History, Properties, Swelling, and Release Mechanism, Material Selection and Applications. Polym. 2017, Vol. 9, Page 137 9, 137 (2017).
Sleight, S. C., Bartley, B. A., Lieviant, J. A. & Sauro, H. M. Designing and engineering evolutionary robust genetic circuits. J. Biol. Eng. 4, 12 (2010).
Tucker, A. T. et al. Discovery of Next-Generation Antimicrobials through Bacterial Self-Screening of Surface-Displayed Peptide Libraries. Cell 172, 618-628 (2018).
Virtanen, P. et al. SciPy 1.0: fundamental algorithms for scientific computing in Python. Nat. Methods 2020 173 17, 261-272 (2020).
Waskom, M. L. seaborn: statistical data visualization. J. Open Source Softw. 6, 3021 (2021).
Wang, X. et al. Bioinspired oral delivery of gut microbiota by self-coating with biofilms. Sci. Adv. 6, eabb1952 (2020).
Zheng, D. W. et al. An orally delivered microbial cocktail for the removal of nitrogenous metabolic waste in animal models of kidney failure. Nat. Biomed. Eng. (2020).
Chinese Patent No. CN105017871B
International Patent Publication No. WO2021026484A1
Japanese Patent No. JP6224277B2
Korean Patent No. KR101465596B1
Korean Patent No. KR102011152B1
U.S. Pat. No. 9,784,730B2
U.S. Ser. No. 10/183,477B2
U.S. Ser. No. 10/786,446B2
U.S. Pat. No. 9,315,669B2
U.S. Pat. No. 9,670,300B2
U.S. Patent Publication No. US20130302252A1
U.S. Patent Publication No. US20060292690A1

1. A device comprising engineered bacteria encapsulated in a hydrogel matrix.

2. The device of claim 1, wherein the engineered bacteria is further defined as an engineered bacterial biosensor.

3. The device of claim 1, wherein the hydrogel matrix comprises alginate.

4. The device of claim 3, wherein the alginate is present at a concentration of 0.1-10 weight percent.

5. The device of claim 1, wherein the hydrogel matrix is paramagnetic.

6. The device of claim 5, wherein the hydrogel matrix further comprises a magnetic small molecule or nanoscale material.

7. The device of claim 6, wherein the nanoscale material is a metal oxide or metal-based agent.

8. The device of claim 7, wherein the metal oxide is titanium oxide, iron oxide, or ferrite.

9. The device of claim 8, wherein the metal-based agent is ferric ammonium citrate.

10. The device of claim 5, wherein the hydrogel matrix further comprises iron oxide.

11. The device of claim 1, wherein the hydrogel matrix further comprises an ultraviolet (UV) absorbent small molecule.

12. The device of claim 11, wherein the UV absorbent small molecule is a dye, photoabsorber, or photoblocker.

13. The device of claim 11, wherein the UV absorbent small molecule is tartrazine (Yellow 5).

14. The device of claim 11, wherein the UV absorbent nanoscale material is a metallic crystal, synthetic polymer, natural polymer, or carbon-based material.

15. The device of claim 11, wherein the UV absorbent nanoscale material is titanium oxide.

16. The device of claim 1, wherein the UV absorbent molecule is attached to alginate.

17. The device of claim 1, wherein the hydrogel matrix is crosslinked with barium cations.

18. The device of claim 1, wherein the hydrogel matrix comprises polyacrylamide, polyethylene glycol (PEG), polyethylene glycol diacrylate (PEGDA), or chitosan.

19. The device of claim 1, wherein the hydrogel matrix comprises alginate and polyacrylamide.

20. The device of claim 19, wherein the polyacrylamide and alginate are present at a ratio of 30:1 to 0.1:1.

21. The device of claim 19, wherein the polyacrylamide and alginate are present at a ratio of 20:1, 10:1, or 5:1.

22. The device of claim 19, wherein the hydrogel matrix comprises alginate and PEGDA.

23. The device of claim 22, wherein the PEGDA and alginate are present at a ratio of 10:1 to 0.5:1.

24. The device of claim 1, wherein the hydrogel comprises tetramethylethylenediamine (TEMED).

25. The device of claim 24, wherein the TEMED is present at a concentration of 0.1% to 30% by volume.

26. The device of claim 24, wherein the TEMED is present at a concentration of 0.1% by volume

27. The device of claim 1, wherein the device comprises an inner core and outer shell.

28. The device of claim 1, wherein the device is in a sphere confirmation.

29. The device of claim 1, wherein the device is in a cylindrical noodle confirmation.

30. The device of claim 27, wherein the engineered bacteria is in the inner core.

31. The device of claim 27, wherein the outer shell is semi-permeable.

32. The device of claim 27, wherein the outer shell comprises a semi-permeable polymer.

33. The device of claim 27, wherein the outer shell is permeable to molecules under 500 kDa.

34. The device of claim 1, wherein the hydrogel matrix is chemically modified.

35. The device of claim 1, wherein the hydrogel matric has a specific density of chemical modifications.

36. The device of claim 35, wherein the modification is an oligosaccharide, bacteriocidal molecule, bacteriostatic molecule, or a DNA vector that is transmitted into the host cell cytoplasm enabling expression of a gene in the host cell.

37. The device of claim 35, wherein the chemical modification has a targeted density between 0 and 5 mmol per gram of alginate in the hydrogel matrix.

38. The device of claim 1, wherein the hydrogel matrix is formulated to promote cell-material interactions between the semi-permeable polymer and the engineered bacteria.

39. The device of claim 38, wherein the matrix has one or more chemical components to promote cell-material interactions.

40. The device of claim 39, wherein the one or more chemical components comprise an adhesive peptide or substrate.

41. The device of claim 40, wherein the adhesive peptide is fibronectin.

42. The device of claim 40, wherein the substrate is xantham gum.

43. The device of claim 2, wherein the engineered bacterial biosensor is genetically engineered to sense a physiological chemical or physical signal.

44. The device of claim 2, wherein the engineered bacterial biosensor responds to one or more biomarkers of intestinal inflammation.

45. The device of claim 44, wherein the biomarker of intestinal inflammation is thiosulfate, tetrathionate, nitrate, trimethyl amine N-oxide, calprotectin, kynurenine, reactive oxygen species, reactive nitrogen species, substance P, CCL20, or acidic pH.

46. The device of claim 2, wherein the engineered bacterial biosensor comprises a one-component cytoplasmic protein capable of sensing ligands in the cytoplasm.

47. The device of claim 46, wherein the one-component cytoplasmic protein capable of sensing ligands in the cytoplasm is SoxS or KynR.

48. The device of claim 2, wherein the engineered bacterial biosensor comprises a membrane-bound two-component system.

49. The device of claim 48, wherein the two-component system comprises ligand-induced signaling and regulation of gene expression by communicating with cytoplasmic proteins.

50. The device of claim 48, wherein the two-component system detects pH, cadaverine, putrescine, nitrite, sialic acid, cellulose, estradiol, testosterone, androstenedione, or human antimicrobial peptides.

51. The device of claim 48, wherein the two-component system is a PhoPQ two-component system.

52. The device of claim 51, wherein the PhoPQ two-component system detects human microbial peptides (AMPs).

53. The device of claim 53, wherein the AMPs are RK-31, KS-27, KS-30, TLN-58, LL-29, ALL-38, GNLY, CCL20, TCP, KR-20, CCL-19, hBD3, and/or NPY.

54. The device of claim 48, wherein the two-component system is a ThsSR, TtrSR, NarXL, TorTSR, PhoPQ, or SO_4387-SO_4388_{REC}-PsdR_{DBD} 137 two-component system.

55. The device of claim 1, wherein the engineered bacteria is an Escherichia coli, Lactobacillus reuteri, Lactobacillus crispatus, Lactobacillus rhamnosus, Lactobacillus crispatus, Lactococcus lactis, Bacteroides thetaiotamicron, or Bacillus subtilis bacterium.

56. The device of claim 1, wherein the engineered bacteria comprises at least two strains of bacteria.

57. The device of claim 56, wherein the at least two strains of bacteria can communicate using acyl homoserine lactones, autoinducer peptides, or other interbacterial communication signals.

58. The device of claim 55, wherein the Escherichia coli is further defined as Escherichia coli Nissle 1917.

59. The device of claim 2, wherein the engineered bacterial biosensor can compute sensory information and respond to the environment outside the construct to activate gene expression directly or activate an engineered genetic circuit that activates gene expression.

60. The device of claim 59, wherein the engineered bacterial biosensor comprises SO_4387-SO_4388_{REC}-PsdR_{DBD} 137 to detect acidic pH and activate expression of a reporter gene or a T7 RNA polymerase to activates production of the reporter gene.

61. The device of claim 2, wherein the engineered bacterial biosensor can respond to a signal by extracellular secretion of a molecule, production of a molecule that diffuses extracellularly, or cell lysis that releases molecules extracellularly.

62. The device of claim 61, wherein the engineered bacterial biosensor secretes IL-22 through protein secretion machinery.

63. The device of claim 62, wherein the protein secretion machinery comprises the Sec pathway, an acylhomoserine lactone that diffuses across the membrane, or the production of a phage lysin that induces cells lysis to result in spillage of IL-22.

64. The device of claim 1, wherein the engineered bacteria expresses a cytokine, anti-NGF, bacteriocin, viral antigen, bacterial antigen, human allergen, monoclonal antibody, nanobody, bacterial quorum sensing molecule, viral vector, DNA, and/or RNA.

65. The device of claim 1, wherein the engineered bacteria expresses at least one reporter.

66. The device of claim 65, wherein the at least one reporter is selected from a chemiluminescent protein, a bioluminescent protein, a fluorescent protein, a colorimetric protein, a pigment-producing enzyme, or a barcoded messenger RNA.

67. The device of claim 65, wherein the at least one reporter is sfGFP.

68. The device of claim 65, wherein the at least one reporter is produced in response to ligand binding.

69. The device of claim 68, wherein the engineered bacteria constitutively expresses a second reporter.

70. The device of claim 1, wherein the engineered bacteria expresses a therapeutic agent.

71. The device of claim 70, wherein the therapeutic agent is a protein.

72. The device of claim 70, wherein the therapeutic agent is not a protein.

73. The device of claim 70, wherein the therapeutic agent is an immune system modulator such as a cytokine, hormone, small molecule, insulin, glucagon, or statin.

74. The device of claim 73, wherein the immune system modulator is a cytokine.

75. The device of claim 74, wherein the cytokine is IL-1, IL-1α, IL-1b, IL-2, IL-3, IL-4, IL-5, IL-6, IL-7, IL-8, IL-9, IL-10, IL-12, IL-15, IL-22, IL-28A, IL-19, IL-24, IL-29, IL-20, IL-28β, TGFβ, IL-12, IL-13, IL-16, IL-18, IFNα, IFNβ, IFNγ, TNFα, TNFβ, or IL-35.

76. The device of claim 70, wherein the engineered bacteria can release the therapeutic agent.

77. The device of claim 76, wherein the therapeutic agent has sustained or conditional release.

78. The device of claim 76, wherein the therapeutic molecule has substantially non-pulsatile release.

79. The device of claim 1, wherein the device comprises 10¹ to 10¹⁰ engineered bacteria.

80. The device of claim 1, wherein each engineered bacteria is about 1 microns to about 3 microns in size.

81. The device of claim 1, wherein the device comprises a degradable component.

82. The device of claim 81, wherein the degradable component is a degradable alginate motif or one or more degradable crosslinkers.

83. The device of claim 82, wherein the degradable alginate motif is partially oxidized alginate.

84. The device of claim 83, wherein the one or more degradable crosslinkers are MMP-sensitive peptide crosslinkers.

85. The device of claim 81, wherein the degradable component is an innate property of the construct material, a product of the material interacting with the in vivo environment, and/or a product of the construct's cellular components interacting directly or indirectly with the construct material.

86. The device of claim 81, wherein the degradable component is in the inner zone and/or outer shell.

87. The device of claim 81, wherein the degradable component is a degradable chemistry motif integrated into the hydrogel matrix as a part of the polymer and/or as a degradable crosslinker.

88. The device of claim 81, wherein the degradable component is degradable is response to pH or an enzyme.

89. The device of claim 81, wherein the degradable is degradable by the engineered bacteria and/or secreted cellular components.

90. The device of claim 1, wherein the device is formulated for oral administration, implantation, inhalation, or injection.

91. The device of claim 1, wherein the device is formulated for implantation.

92. A method of treating a disease in a subject in need thereof comprising administering an effective amount of the device of claim 1 to the subject.

93. The method of claim 92, wherein the disease is inflammatory bowel disease, Crohn's, ulcerative colitis, vaginal yeast infection, bacterial vaginosis, parasitic infection, appendicitis, lung cancer, acute respiratory distress syndrome, mesothelioma, thrush, or oral cancer.

94. The method of claim 93, wherein the parasitic infection trichomoniasis.

95. The method of claim 92, wherein the disease is Crohn's.

96. The method of claim 92, wherein the disease is inflammatory bowel disease.

97. The method of claim 92, wherein the disease is ulcerative colitis.

98. The method of claim 92, further comprising monitoring inflammation in the subject.

99. The method of claim 98, wherein monitoring inflammation comprises detecting a reporter produced by the engineered bacteria.

100. The method of claim 86, wherein monitoring inflammation does not comprise an invasive procedure.

101. The method of claim 100, wherein the invasive procedure is a colon biopsy or endoscopy.

102. The method of claim 98, wherein monitoring inflammation comprises analysis of a fecal sample from said subject and performing flow cytometry to detect said reporter.

103. A method of monitoring inflammation in a subject in need thereof comprising administering an effective amount of the device of claim 1 to the subject and detecting a reporter produced by the engineered bacteria.

104. The method of claim 103, wherein the subject has inflammatory bowel disease.

105. The method of claim 103, wherein the subject has Crohn's or ulcerative colitis.

106. The method of claim 103, wherein monitoring inflammation does not comprise an invasive procedure.

107. The method of claim 106, wherein the invasive procedure is a colon biopsy or endoscopy.

108. The method of claim 103, wherein detecting comprises analysis of a fecal sample from said subject and performing flow cytometry to determine the level of said reporter.
